(19) United States
(12) Patent Application Publication
(10) Pub. No.: US 2018/0303877 A1
(43) Pub. Date: Oct. 25, 2018

(54) CELL-BASED TARGETED DELIVERY OF PSEUDONOMAS EXOTOXIN

(71) Applicant: THE GENERAL HOSPITAL CORPORATION, Boston, MA (US)

(72) Inventor: Khalid Shah, Andover, MA (US)

(73) Assignee: THE GENERAL HOSPITAL CORPORATION, Boston, MA (US)

(21) Appl. No.: 15/513,385

(22) PCT Filed: Sep. 25, 2015
(86) PCT No.: PCT/US15/52322

§ 371 Date: Mar. 22, 2017

Related U.S. Application Data
(60) Provisional application No. 62/055,177, filed on Sep. 25, 2014.

(51) Int. Cl. A61K 35/28; C12N 15/85; A61K 35/30; A61K 35/545; C07K 14/21; C07K 14/54; A61K 48/00; A61P 35/00

(52) U.S. Cl. CPC A61K 35/28; C12N 15/85; A61K 35/30; A61P 35/00; C07K 14/21; C07K 14/5437; A61K 48/0016; A61K 35/545

(57) ABSTRACT

Embodiments herein provide engineered mammalian cells, compositions comprising these cells and methods for targeted cancer therapy using cytotoxins derived from Pseudonomas sp. Specifically, it relates to cell-based, targeted in vivo delivery of Pseudonomas exotoxin (PE) for cancer therapy.

### CROSS REFERENCE TO RELATED APPLICATION

This International PCT Application claims benefit under 35 U.S.C. § 119(e) of U.S. Provisional Application No. 62/055,177 filed on Sep. 25, 2015, the contents which are incorporated herein by reference in their entireties.

### GOVERNMENT SUPPORT

This invention was made with Government support under Grant No.: RO1 CA138922-04 and RO1 CA173077 awarded by the National Institutes of Health. The Government has certain rights in the invention.

### FIELD OF THE DISCLOSURE

The disclosure relates to cells, compositions and methods for targeted cancer therapy using cytotoxins derived from Pseudonomas sp. Specifically, it relates to cell-based, targeted in vivo delivery of Pseudonomas exotoxin for cytotoxic therapy, e.g. cancer therapy.

### BACKGROUND OF THE DISCLOSURE

Pseudomonas exotoxin (PE) is a single, multi-domain peptide with the ability to enter cells and kill them by catalyzing the inactivation of elongation factor-2 (EF-2), thereby blocking protein synthesis. In order to direct PE specifically to the cancer cells, a multitude of antibody variable fragments (Fv) and ligands directed against cancerous cells have been fused to PE. Many human cancers, including >50% of glioblastoma multiforme (GBM), express a variant form of the IL-13 receptor called IL-13Rα2, permitting high affinity binding of IL13-PE (3-7). Normal brain cells do not express IL-13Rα2, thus providing a rationale to selectively target and kill GBM cells. Epidermal growth factor receptor (EGFR) is also overexpressed and mutated in a variety of tumors, including GBM, and much effort has been channeled into developing PE-conjugated fusion proteins that target EGFR on malignant cells.

In the clinic, PE-based cytotoxins have been used with great success to treat a variety of hematologic malignancies including leukemia and Hodgkin's lymphoma. Yet, attaining similar results in solid tumors has been hindered by inadequate distribution of the cytotoxin throughout the tumor mass coupled to the relatively short half-life of PE. Preclinical testing demonstrated that IL13-PE was highly toxic in culture and in vivo towards IL-13Rα2-expressing cells, and early phase clinical trials reported that despite some adverse effects, IL13-PE was well tolerated and appeared to have a favorable risk-benefit profile. However, in spite of great expectations, the Phase III PRECISE clinical trial failed to show a significant survival benefit in patients with recurrent GBM. The failure of this study was likely due to the short half-life of IL13-PE coupled to ineffective delivery of the toxin to residual GBM cells following surgical resection.

### SUMMARY OF THE DISCLOSURE

Embodiments of the disclosure are based on a new approach that would circumvent the short half-life of IL13-PE in circulation in a subject. Instead of administering purified IL13-PE systematically into a subject, the new approach is to administer specially engineered cells into the subject, these engineered cells are designed to be resistant to the Pseudonomas exotoxin (PE), and also are engineered to express and secrete IL13-PE. This approach ensures an in vivo, cell-based, sustained amount of IL13-PE for therapy as long as the engineered cells remain viable in the subject.

Accordingly, in one embodiment, this disclosure provides an engineered mammalian cell that is resistant to PE and also expresses and secretes PE. In one embodiment, the cell comprises a guanosine to adenosine (G→A) mutation in the first nucleotide of codon 717 of a coding sequence of an elongation factor 2 (EF-2) protein therein and an exogenous nucleic acid sequence encoding for a protein comprising a PE or a fragment thereof.

In one embodiment, this disclosure provides an engineered mammalian cell that is resistant to PE and also expresses and secretes PE for use in the treatment of cancer. In one embodiment, the cell comprises a G→A mutation in the first nucleotide of codon 717 of a coding sequence of an EF-2 protein therein and an exogenous nucleic acid sequence encoding a protein comprising a PE or a fragment thereof.

In one embodiment, this disclosure provides an engineered mammalian cell that is resistant to PE and also expresses and secretes PE for use in the manufacture of medicament for the treatment of cancer. In one embodiment, the cell comprises a G→A mutation in the first nucleotide of codon 717 of a coding sequence of an EF-2 protein therein and an exogenous nucleic acid sequence encoding for a protein comprising a PE or a fragment thereof.

In one embodiment, this disclosure provides a composition comprising of engineered mammalian cells that are resistant to PE and also express and secrete PE. In one embodiment, the cell comprises a G→A mutation in the first nucleotide of codon 717 of a coding sequence of an EF-2 protein therein and an exogenous nucleic acid sequence encoding for a protein comprising a PE or a fragment thereof. In one embodiment, the composition further comprises a pharmaceutically acceptable carrier. In another embodiment, the composition further comprises an extracellular matrix and/or a scaffold material.

In one embodiment, this disclosure provides a composition comprising of engineered mammalian cells that are resistant to PE and also express and secrete PE for use in the treatment of cancer. In one embodiment, the cell comprises a G→A mutation in the first nucleotide of codon 717 of a coding sequence of an EF-2 protein therein and an exogenous nucleic acid sequence encoding for a protein comprising a PE or a fragment thereof.

In one embodiment, this disclosure provides a composition comprising of engineered mammalian cells that are resistant to PE and also express and secrete PE for use in the manufacture of medicament for the treatment of cancer. In one embodiment, the cell comprises a G→A mutation in the first nucleotide of codon 717 of a coding sequence of an EF-2 protein therein and an exogenous nucleic acid sequence encoding for a protein comprising a PE or a fragment thereof.

In one embodiment, this disclosure provides a method of making an engineered mammalian cell that is resistant to Pseudonomas exotoxin or diphtheria toxin or a ADP-ribosylating toxin comprising: (a) contacting a mammalian cell with a nucleic acid sequence that confers a G→A mutation in the first nucleotide of codon 717 of a coding sequence of an EF-2 protein; (b) culturing the mammalian cell of step (a), after the contacting, in the presence of a ADP-ribosylating toxin for a period of time; and (c) selecting for the mammalian cell of step (b) that has formed a single colony in the presence of the ADP-ribosylating toxin. In other words, selecting for viable engineered cells in the presence of the ADP-ribosylating toxin, and collecting these viable, ADP-ribosylating toxin-resistant engineered cells for transfection with an exogenous nucleic acid sequence encoding for a protein comprising a PE or a fragment thereof. The conferred resistance now allows these cells to manufacture PE or a protein comprising a PE or a fragment thereof in vivo, and not succumb to the PE cytotoxic effects.

In one embodiment of any aspects disclosed herein, the method further comprising: (e) contacting the colony of cells of step (c) with a vector comprising an exogenous nucleic acid sequence encoding for a Pseudonomas exotoxin (PE) or a fusion protein comprising PE or a fragment thereof; (f) culturing the contacted cell of step (d) for a period of time; and (g) selecting for the contacted cell of step (d) for the expression of PE or the fusion protein.

In another embodiment, the colony of cells of step (c) with a vector comprising an exogenous nucleic acid sequence encoding for a diphtheria toxin or fragment thereof or a ADP-ribosylating toxin, (f) culturing the contacted cell of step (d) for a period of time; and (g) selecting for the contacted cell of step (d) for the expression of the diphtheria toxin or fragment thereof or the ADP-ribosylating toxin.

In one embodiment of any aspects disclosed herein, the method further comprising collecting and harvesting the colony of cells of step (c) prior to the contacting step (d).

In one embodiment of any aspects disclosed herein, the method further comprising culture expansion of the colony of cells of step (c) prior to the contacting step (d).

In one embodiment of any aspects disclosed herein, the method further comprising collecting and harvesting viable engineered cells that express the PE, the PE fusion protein, diphtheria toxin or fragment thereof or the ADP-ribosylating toxin.

In one embodiment, this disclosure provides a method of making a self-renewing engineered mammalian cell that secretes Pseudonomas exotoxin (PE) or a protein comprising PE, or a DT or a fusion protein comprising DT, the method comprising: (a) providing an engineered mammalian cell that is resistant to Pseudonomas exotoxin, or diphtheria toxin or or a ADP-ribosylating toxin; (b) contacting the PE/DT-resistant cell with a vector comprising an exogenous nucleic acid sequence encoding for a PE or a fusion protein comprising PE or a fragment thereof, or with a vector comprising an exogenous nucleic acid sequence encoding for a DT or a fusion protein comprising DT or a fragment thereof; (c) culturing the contacted cell of step (b) for a period of time; and (d) selecting for the cell of step (c) for the expression of PE/DT or PE/DT-fusion protein.

In one embodiment, this disclosure provides a method of making a self-renewing engineered mammalian cell that secretes PE, or a protein comprising PE, or a DT or a fusion protein comprising DT, the method comprising: (a) contacting a mammalian cell that is resistant to Pseudonomas exotoxin, or diphtheria toxin or a ADP-ribosylating toxin with a vector comprising an exogenous nucleic acid sequence encoding for a PE or a fusion protein comprising PE or a fragment thereof, or with a vector comprising an exogenous nucleic acid sequence encoding for a DT or a fusion protein comprising DT or a fragment thereof; (b) culturing the contacted cell of step (a) for a period of time; and (c) selecting for the cell of step (b) for the expression of PE/DT or PE/DT-fusion protein.

In one embodiment, this disclosure provides a method of treating cancer in a subject comprising administering an effective amount of engineered mammalian cells described herein or a composition comprising the engineered mammalian cells described herein into the subject. In one embodiment, the cell comprises a G→A mutation in the first nucleotide of codon 717 of a coding sequence of an EF-2 protein therein and an exogenous nucleic acid sequence encoding for a PE or a fusion protein comprising PE or a fragment thereof. In another embodiment, the cell comprises a G→A mutation in the first nucleotide of codon 717 of a coding sequence of an EF-2 protein therein and an exogenous nucleic acid sequence encoding for a DT or a fusion protein comprising DT or a fragment thereof.

In one embodiment, this disclosure provides a method of inducing apoptosis in a tumor or cancer cells in a subject comprising: (a) resectioning a tumor from the subject; and (b) implanting a population of engineered mammalian cells described herein or a composition comprising the engineered mammalian cells described herein into the resection site produced in step (a).

In one embodiment, this disclosure provides a method for studying the in vivo cytotoxic effectiveness of an engineered cell expressing a cytotoxin, the method comprising: (a) providing an animal model comprising a cancer in vivo; (b) implanting a population of engineered mammalian cells described herein or a composition comprising the engineered mammalian cells described herein into the animal model; and (c) monitoring the growth of the cancer in the animal model.

In one embodiment, this disclosure provides a method of sustained in vivo delivery of Pseudonomas exotoxin or a fusion protein comprising PE or a fragment thereof in a subject comprising administering an effective amount of engineered mammalian cells of described herein or a composition comprising the engineered mammalian cells described herein into the subject.

In one embodiment, this disclosure provides a method of sustained in vivo delivery of a diphtheria toxin or a fusion protein comprising DT or a fragment thereof in a subject comprising administering an effective amount of engineered mammalian cells of described herein or a composition comprising the engineered mammalian cells described herein into the subject.

In one embodiment of any aspects disclosed herein, the cell comprises a G→A mutation in the first nucleotide of codon 717 of a coding sequence of an EF-2 protein therein and an exogenous nucleic acid sequence encoding for a PE or a fusion protein comprising PE or a fragment thereof.

In one embodiment, this disclosure provides an engineered mammalian cell that is resistant to PE. In one embodiment, the engineered cell comprises a G→A mutation in the first nucleotide of codon 717 of a coding sequence of an elongation factor 2 protein (EF-2) therein.

In one embodiment, this disclosure provides a composition comprising engineered mammalian cells that are resistant to PE. In one embodiment, the cells of the composition also synthesize in vivo PE or a fusion protein comprising PE or a fragment thereof. In another embodiment, the cells of the composition also synthesize in vivo DT or a fusion protein comprising DT or a fragment thereof. In one embodiment, the cell comprises a G→A mutation in the first nucleotide of codon 717 of a coding sequence of an EF-2 protein therein. In one embodiment, the composition further comprises a pharmaceutically acceptable carrier.

In one embodiment of any aspects disclosed herein, the engineered mammalian cell comprises a modified nucleic acid sequence encoding an EF-2.

In one embodiment of any aspects disclosed herein, the modified nucleic acid sequence encoding the EF-2 has a G→A mutation in the first nucleotide of codon 717 of a protein coding sequence for EF-2.

In one embodiment of any aspects disclosed herein, the EF-2 protein is a human EF-2 protein.

In one embodiment of any aspects disclosed herein, the nucleic acid sequence that confers a G→A mutation in the first nucleotide of codon 717 of a coding sequence of an EF-2 protein comprises an oligonucleotide that is at least 90% of SEQ. ID. No: 1, tgtggggatctggccccctctgcggtggatggcgtcggcgtgca.

In one embodiment of any aspects disclosed herein, the nucleic acid sequence that confers a G→A mutation in the first nucleotide of codon 717 of a coding sequence of an EF-2 protein comprises an oligonucleotide that is about 40 to about 60 nucleotides long in length and the oligonucleotide is exactly identical to the coding sequence of an EF-2 protein except for the single nucleotide G→A change in the first nucleotide of codon 717 of the coding sequence of the EF-2 protein.

In another embodiment, the oligonucleotide is about 47 nucleotides long and is SEQ. ID. NO: 1.

In one embodiment of any aspects disclosed herein, the engineered mammalian cell described herein expresses a mutant EF-2 protein.

In one embodiment of any aspects disclosed herein, the mutant EF-2 protein is resistant to ADP-ribosylation on a conserved histidine residue.

In one embodiment of any aspects disclosed herein, the mutant EF-2 protein is resistant to ADP-ribosylation on the conserved histidine residue located at the amino acid position 710, 696, 694 or 704 of the EF-2 polypeptide, depending on the species source of the EF-2 protein. For example, in the human and hamster EF-2, the conserved histidine residue located at the amino acid position 710.

In one embodiment of any aspects disclosed herein, the engineered mammalian cell comprises an exogenous nucleic acid sequence that encodes for a PE or a fusion protein comprising PE or a fragment thereof.

In one embodiment of any aspects disclosed herein, the engineered mammalian cell comprises an exogenous nucleic acid sequence that encodes for a DT or a fragment thereof.

In one embodiment of any aspects disclosed herein, the engineered mammalian cell expresses a protein comprising PE. In one embodiment, the expressed protein is a fusion protein comprising PE.

In one embodiment of any aspects disclosed herein, the engineered mammalian cell expresses a protein comprising a DT. In one embodiment, the expressed protein is a fusion protein comprising a DT.

In one embodiment of any aspects disclosed herein, the engineered mammalian cell secretes a protein comprising PE. In one embodiment, the secreted protein is a fusion protein comprising PE.

In one embodiment of any aspects disclosed herein, the engineered mammalian cell secretes a protein comprising a DT. In one embodiment, the secreted protein is a fusion protein comprising a DT.

In one embodiment of any aspects disclosed herein, the expressed PE is a fusion protein.

In one embodiment of any aspects disclosed herein, the fusion protein further comprises interleukin 13 (IL-13).

In one embodiment of any aspects disclosed herein, the fusion protein further comprises epidermal growth factor (EGF).

In one embodiment of any aspects disclosed herein, the mammalian cell is a somatic cell.

In one embodiment of any aspects disclosed herein, the mammalian cell is a stem cell.

In one embodiment of any aspects disclosed herein, the stem cell is selected from a group consisting of neural stem cells, (NSCs), mesenchymal stem cells (MSCs) derived from bone marrow, adipose tissue, cord blood and dental pulp, induced pluripotent stem cells (iPSCs) and iPSC-derived NSC, T cells and MSCs.

In one embodiment of any aspects disclosed herein, the exogenous nucleic acid sequence encodes a fusion PE protein or a fusion protein comprising a diphtheria toxin.

In one embodiment of any aspects disclosed herein, the exogenous nucleic acid sequence encodes a fusion PE that is destined for extracellular secretion.

In one embodiment of any aspects disclosed herein, the exogenous nucleic acid sequence comprising any one of SEQ. ID. NOS: 2, 3, 4, 17 or 18.

In one embodiment of any aspects disclosed herein, the ADP-ribosylating toxin is diphtheria or PE.

In one embodiment of any aspects disclosed herein, the method further comprising selecting a subject who has been diagnosed with cancer.

In one embodiment of any aspects disclosed herein, the method further comprising analysis of the cancer cells from the subject for the expression of interleukin 13 alpha 2 receptor (IL-13Rα2) and/or epidermal growth factor receptor (EGFR).

In one embodiment of any aspects disclosed herein, the cancer cells express moderate to high amount of IL-13Rα2.

In one embodiment of any aspects disclosed herein, the cancer cells express moderate to high amount of EGFR.

In one embodiment of any aspects disclosed herein, the cancer cell expresses the mutant EGFR variant (EGFR vIII).

In one embodiment of any aspects disclosed herein, the engineered mammalian cells express and secret a fusion EGF-PE when the cancer expresses moderate to high amount of EGFR or expresses EGFR vIII.

In one embodiment of any aspects disclosed herein, the engineered mammalian cells express and secret a fusion IL-13-PE when the cancer expresses moderate to high amount of IL-13Rα2.

In one embodiment of any aspects disclosed herein, the cancer comprises solid tumors.

In one embodiment of any aspects disclosed herein, the cancer is selected from a group consisting of brain tumors such as glioblastoma multiforme (GBM), melanoma, breast cancer, and lung cancer.

In one embodiment of any aspects disclosed herein, the engineered mammalian cells are administered systemically.

In one embodiment of any aspects disclosed herein, the engineered mammalian cells are administered directly to, locally or near the cancer cells in the subject.

In one embodiment of any aspects disclosed herein, the population of engineered mammalian cells is encapsulated prior to implantation.

In one embodiment of any aspects disclosed herein, the tumor resection is total or partial.

In one embodiment of any aspects disclosed herein, the method further comprising implanting a population of engineered mammalian cells described herein into the periphery of the resection site.

In one embodiment of any aspects disclosed herein, the method further comprising implanting an additional cancer therapy in the vicinity of the resection site. In one embodiment, the additional cancer therapy does not comprise engineered cells that expresses PE or are resistant to PE.

In one embodiment of any aspects disclosed herein, the cytotoxin is Pseudonomas exotoxin A (PE). In one embodiment of any aspects disclosed herein, the cytotoxin is diphtheria toxin (DT). In one embodiment, the diphtheria toxin is the fragment A toxin.

In one embodiment of any aspects disclosed herein, the cancer forms solid tumors in the animal.

In one embodiment of any aspects disclosed herein, the treatment method further comprising resectioning the solid tumors prior to implanting said cells.

### Definitions

As used herein, the term “PE” refers to a Pseudonomas exotoxin A protein. In one embodiment, PE refers to a full-length Pseudonomas exotoxin protein. The full length PE protein as described in UniProKB-P11439 (TOXA_PSEAE), and in GENBANK Accession No: gi|151216|gb|AAB59097.1 where the protein has 1-638 amino acid residues (SEQ. ID. NO: 21). In another embodiment, PE refers to a fragment of the full-length Pseudonomas exotoxin protein, that is, containing less than the 638 amino acid residues. For examples, a fragment of the full-length Pseudonomas exotoxin can be the region of amino acid residues 26-638 (without the signal peptide), the region of amino acid residues 26-277 (domain IA, required for target cell recognition), amino acid residues 278-389 (domain II, required for translocation in the target cell cytoplasm), amino acid residues 430-638 (domain III, required for ADP-ribosyl activity), SEQ. ID. NO: 20 or 23.

As used herein, the term “DT” refers to a diphtheria toxin protein. In one embodiment, DT refers to a full-length diphtheria toxin protein. The full length diphtheria toxin protein as described in UniProtKB—P00588 (DTX_CORBE) with 1-567 amino acid residues. In another embodiment, DT refers to a fragment of a full-length diphtheria toxin protein, that is, containing less than 567 amino acid residues. For examples, a fragment of a full-length diphtheria toxin protein can be the region of amino acid residues 33-567 (without the signal peptide), the region of amino acid residues 33-225 (the diphtheria toxin fragment A) and the region of amino acid residues 226-567 (the diphtheria toxin fragment B).

As used herein, the phrase “resistant to PE” when used in the context of an engineered mammalian cell refers to the cell not being affected by the cytotoxic effects of PE, In another embodiment, the “resistant to PE” also means that such resistant cell is not affected by the cytotoxic effects of DT, i.e., the engineered mammalian cell does not undergo cell death or apoptosis in the presence of PE or DT. In one embodiment, a cell that is able to synthesize protein in vivo in the presence of PE or DT. In another embodiment, “resistant to PE” refers to the cell having a modified EF-2 protein that is not ADP ribosylated by PE or DT, thus the modified EF-2 protein remain functional and the cell is able to synthesize protein in vivo in the presence of PE or DT.

As used herein, the phrase “secretes PE” when used in the context of an engineered mammalian cell refers to the cell synthesizing in vivo a PE protein or a protein comprising a PE, and exporting the PE protein extracellular to outside the cell.

As used herein, the clause “a protein comprising a PE” when used in the context of the exogenous nucleic acid sequence encoding a protein refers to a polypeptide that contains the Pseudonomas exotoxin A protein or a fragment of the Pseudonomas exotoxin protein. For example, “a protein comprising a PE” is a polypeptide containing only the full length PE protein as described in UniProKB-P11439 (TOXA_PSEAE) with 1-638 amino acid residues (SEQ. ID. NO: 21). This full length PE includes the signal peptide located at amino acid residues 1-25 and the exotoxin A at located amino acid residues 26-638. In other embodiments, the protein comprising a PE can be polypeptide containing only a fragment or domain of the full length PE protein as described in UniProKB-P11439, i.e., containing less than the 638 amino acid residues. For example, the region of amino acid residues 26-638 (without the signal peptide), the region of amino acid residues 26-277 (domain IA, required for target cell recognition), amino acid residues 278-389 (domain II, required for translocation in the target cell cytoplasm), and amino acid residues 430-638 (domain III, required for ADP-ribosyl activity). In one embodiment, a protein comprising PE comprises SEQ. ID. NO: 20 or 23. In one embodiment, the polypeptide can be a chimeric or fusion polypeptide containing PE amino acid sequences and also contain amino acid sequence of another not PE protein.

As used herein, the clause “a protein comprising a diphtheria toxin” when used in the context of the exogenous nucleic acid sequence encoding a protein refers to a polypeptide that contains the diphtheria toxin protein or a fragment of the diphtheria toxin protein. For example, “a protein comprising a diphtheria toxin” is a polypeptide containing only the full length diphtheria toxin protein as described in UniProtKB—P00588 (DTX_CORBE) with 1-567 amino acid residues. This full length diphtheria toxin includes the signal peptide located at amino acid residues 1-32, the diphtheria toxin fragment A located at amino acid residues 33-225, and the diphtheria toxin fragment B located amino acid residues 226-567. In other embodiments, the protein comprising a diphtheria toxin can be polypeptide containing only a fragment or domain of the full length diphtheria toxin protein as described in UniProKB-P00588, i.e., containing less than the 567 amino acid residues. For example, the region of amino acid residues 33-567 (without the signal peptide), the region of amino acid residues 33-225 (the diphtheria toxin fragment A) and the region of amino acid residues 226-567 (the diphtheria toxin fragment B). In one embodiment, the polypeptide can be a chimeric or fusion polypeptide containing diphtheria toxin amino acid sequences and also contain amino acid sequence of another not diphtheria toxin protein.

The term “fragment” or “fragment thereof” refers to any subject polypeptide having an amino acid residue sequence shorter than that of a polypeptide whose amino acid residue sequence of the full-length protein described herein. For example, a fragment of PE is the regions of amino acid residues 26-638, 85-201, 430-638, 431-638, 433-638, and 434-638 of the full-length PE in SEQ. ID. NO: 21. In one embodiment, a fragment of PE represents the domain III that required for ADP-ribosyl activity, in regions 430-638, 431-638, 433-638, and 434-638 of the full-length PE. For example, a fragment of diphtheria toxin is the regions of amino acid residues 33-255, 35-255, 34-255, and 36-255 representing the diphtheria toxin fragment A that is active in ADP-ribosylation of EF-2 protein.

As used herein, the term “fusion” when used in the context of a fusion PE protein protein or a fusion diphtheria toxin protein refers to a protein created by joining two genes or two proteins/peptides together. The two proteins are not the same proteins. In the laboratory, this is achieved through the creation of a fusion gene which is done through the removal of the stop codon from a DNA sequence of the first protein and then attaching the DNA sequence of the second protein in frame. The resulting DNA sequence will then be expressed by a cell as a single protein. In a fusion protein, the two proteins that will be joined together with a linker or spacer peptide added between the two proteins. This linker or spacer peptide often contain protease cleavage site to facilitate the separation of the two proteins after expression and purification. The making of fusion protein as a technique is commonly used for the identification and purification of proteins through the fusion of a GST protein, FLAG peptide or a hexa-his peptide. In one embodiment, a “fusion protein” refers to a recombinant protein of two or more proteins which are joined by a peptide bond. Fusion proteins can be produced, for example, by a nucleic acid sequence encoding one protein joined to a nucleic acid encoding another protein such that they constitute a single open-reading frame that can be translated into a single polypeptide harboring each of the intended proteins or protein domains.

The terms “identical”, in the context of two or more nucleic acid or polypeptide sequences, refers to two or more sequences or subsequences that are the same over a specified region (e.g., nucleotide sequence encoding an EF-2 protein described herein or amino acid sequence of an EF-2 protein described herein), when compared and aligned for maximum correspondence over a comparison window or designated region) as measured using a BLAST or BLAST 2.0 sequence comparison algorithms with default parameters described below, or by manual alignment and visual inspection. This term also refers to, or can be applied to, the complement of a test sequence. The term also includes sequences that have deletions and/or additions, as well as those that have substitutions. Preferred algorithms can account for gaps and the like. Computer programs for the determination of the identity between two sequences include, but are not restricted to, the GCG programme package, including GAP (Devereux et al., 1984); Genetics Computer Group University of Wisconsin, Madison, (Wis.)); BLASTP, BLASTN and FASTA (Altschul et al., NCB NLM NIH Bethesda Md. 20894; Altschul et al., 1990). The well-known Smith Waterman algorithm can also be used for the determination of identity.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present disclosure can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques. Suitable pharmaceutical carriers are described in Remington's Pharmaceutical Sciences, A. Osol, a standard reference text in this field of art. For example, a parenteral composition suitable for administration by injection is prepared by dissolving 1.5% by weight of active ingredient in 0.9% sodium chloride solution.

The term “carrier” in the context of a pharmaceutical carrier refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration.

The term “pharmaceutically acceptable carriers” excludes tissue culture medium.

As used herein, the terms “administering” refers to the placement of the described engineered mammalian cells or the composition comprising the described engineered mammalian cells into a subject in need thereof by a method or route which results in at least partial localization of the described engineered mammalian cells at a desired site. The described engineered mammalian cells or the composition comprising the described engineered mammalian cells can be administered by any appropriate route which results in an effective treatment in the subject. In one embodiment, “administering” refers to direct implanting or transplanting the described engineered mammalian cells into the subject.

The term “effective amount” means an amount of described engineered mammalian cells sufficient to provide at least some amelioration of the symptoms associated with cancer. In one embodiment, the “effective amount” means an amount of described engineered mammalian cells would decrease the respective cancer markers in a subject having cancer. In another embodiment, the “effective amount” means an amount of described engineered mammalian cells would induce cell death in cancer cells in an afflicted subject such that the cancer or tumor reduces in size.

As used herein, the term “comprising” or “comprises” is used in reference to methods and compositions, and respective component(s) thereof, that are essential to the invention, yet open to the inclusion of unspecified elements, whether essential or not. The use of “comprising” indicates inclusion rather than limitation.

The described engineered mammalian cells can be autologous/autogeneic (“self”) or non-autologous (“non-self,” e.g., allogeneic, syngeneic or xenogeneic). “Autologous,” as used herein, refers to cells from the same subject.

“Allogeneic,” as used herein, refers to cells of the same species that differ genetically to the cell in comparison.

“Syngeneic,” as used herein, refers to cells of a different subject that are genetically identical to the cell in comparison.

“Xenogeneic,” as used herein, refers to cells of a different species to the cell in comparison.

A “subject,” as used herein, includes any animal that exhibits a symptom of hypothyroidism described herein. Suitable subjects (e.g., patients) include laboratory animals (such as mouse, rat, rabbit, or guinea pig), farm animals, and domestic animals or pets (such as a cat or dog). Non-human primates and, preferably, human patients, are included. In another embodiment, the subject is a human.

In one embodiment, as used herein “treatment” or “treating,” includes any beneficial or desirable effect on the symptoms of cancer, and may include even increase in the amount of cancer markers in circulation in the subject. In another embodiment, treatment can involve optionally either the reduction or amelioration of symptoms of the disease or condition. “Treatment” does not necessarily indicate complete eradication or cure of the disease or condition, or associated symptoms thereof.

The term “stem cell” refers then, to a cell with the capacity or potential, under particular circumstances, to differentiate to a more specialized or differentiated phenotype, and which retains the capacity, under certain circumstances, to proliferate without substantially differentiating. In one embodiment, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a pluripotent cell which itself is derived from a pluripotent cell, and so on. While each of these pluripotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “pluripotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Generally, “progenitor cells” have a cellular phenotype that is more primitive (i.e., is at an earlier step along a developmental pathway or progression than is a fully differentiated cell). Often, progenitor cells also have significant or very high proliferative potential. Progenitor cells can give rise to multiple distinct differentiated cell types or to a single differentiated cell type, depending on the developmental pathway and on the environment in which the cells develop and differentiate.

As used herein, the term “a progenitor cell” refers to refer to an immature or undifferentiated cell that has the potential later on to mature (differentiate) into a specific cell type, for example, a blood cell, a skin cell, a bone cell, or a hair cells. A progenitor cell also can proliferate to make more progenitor cells that are similarly immature or undifferentiated.

As used herein, the term “vector” refers broadly to any plasmid, phagemid or virus encoding an exogenous nucleic acid. The term is also be construed to include non-plasmid, non-phagemid and non-viral compounds which facilitate the transfer of nucleic acid into virions or cells, such as, for example, poly-lysine compounds and the like. The vector may be a viral vector that is suitable as a delivery vehicle for delivery of the nucleic acid, or mutant thereof, to a cell, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94: 12744-12746). Examples of viral vectors include, but are not limited to, a recombinant Vaccinia virus, a recombinant adenovirus, a recombinant retrovirus, a recombinant adeno-associated virus, a recombinant avian pox virus, and the like (Cranage et al., 1986, EMBO J. 5: 3057-3063; International Patent Application No. WO94/17810, published Aug. 18, 1994; International Patent Application No. WO94/23744, published Oct. 27, 1994). Examples of non-viral vectors include, but are not limited to, liposomes, polyamine derivatives of DNA, and the like.

An “expression vector” refers to a vector that has the ability to incorporate and express exogenous DNA fragments in a foreign cell. A cloning or expression vector may comprise additional elements, for example, the expression vector may have two replication systems, thus allowing it to be maintained in two organisms, for example in human cells for expression and in a prokaryotic host for cloning and amplification. The term vector may also be used to describe a recombinant virus, e.g., a virus modified to contain the coding sequence for a therapeutic compound or factor. As used herein, a vector may be of viral or non-viral origin.

As used herein, the term “viral vector” refers to a nucleic acid vector construct that includes at least one element of viral origin and has the capacity to be packaged into a viral vector particle. The viral vector can contain the protein coding sequence for IL-13-PE or PE or ENb-PE in place of non-essential viral genes. The vector and/or particle may be utilized for the purpose of transferring any nucleic acids into cells either in vitro or in vivo. Numerous forms of viral vectors are known in the art.

As used herein, the term “lentivirus” refers to a group (or genus) of retroviruses that give rise to slowly developing disease. Viruses included within this group include HIV (human immunodeficiency virus; including HIV type 1, and HIV type 2), the etiologic agent of the human acquired immunodeficiency syndrome (AIDS); visna-maedi, which causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which cause immune deficiency and encephalopathy in sub-human primates. Diseases caused by these viruses are characterized by a long incubation period and protracted course. Usually, the viruses latently infect monocytes and macrophages, from which they spread to other cells. HIV, FIV, and SIV also readily infect T lymphocytes, i.e., T-cells.

In one embodiment, the term “lentiviral vector” refers to a vector having a nucleic acid vector construct that includes at least one element of lentivirus origin.

### BRIEF DESCRIPTION OF THE DRAWINGS

FIGS. 1A-1H. Engineering toxin-resistant stem cells that secrete PE-cytotoxins.

FIG. 1A. Schematic representation of the approach used to make cells resistant to PE-immunotoxins. Wild-type cells were transfected with the single stranded oligonucleotide, ssODN-mEF-2, to introduce a mutation in the endogenous EF-2 gene. Cells were cultured in toxin-containing media, and single resistant clones were selected and expanded.

FIG. 1B. Summary data showing the growth rates of SO, hNSC, SO-Oligo, and hNSC-Oligo cells at day 1, 2, 5 and 10 days.

FIG. 1C. Summary graph demonstrating the viability of SO, hNSC, SO-Oligo, and hNSC-Oligo cells treated with DT at increasing concentrations (0-1000 ng/mL).

FIG. 1D. Schematic representation of the approach for introducing cytotoxins into toxin-resistant cells. Resistant clones were transfected with a vector encoding IL13-PE cloned upstream of a fluorescence marker and puromycin selection cassette. Cells were cultured in the presence of puromycin (1 ug/mL) and positive, puromycin resistant clones were selected, expanded and characterized.

FIG. 1E. Toxin resistant hNSC-Oligo cells were engineered to stably express IL13-PE-mCherry or mCherry alone. The arrangement of the construct of the expression vector is shown.

FIG. 1F. Western blot analysis demonstrating IL13-PE protein expression in the lysates of hNSC-Oligo cells stably expressing IL13-PE.

FIG. 1G. Toxin resistant hNSC-Oligo cells were engineered to stably express ENb-PE-eGFP or eGFP alone. The arrangement of the construct of the expression vector is shown.

FIG. 1H. RT-PCR demonstrating the presence of PE transcript expression in hNSC-ENb-PE cells versus unmodified hNSC cells.

FIGS. 2A-2G. Stem cell-delivered PE-cytotoxins reduce cell viability of GBMs

FIG. 2A. Western blot analysis of IL13Rα2 expression from the lysates of established human GBM lines.

FIG. 2B. Cell viability of human GBM cells expressing eGFP-Fluc, co-cultured with hNSC-IL13-PE-mCherry or bNSC-mCherry.

FIG. 2C. Lentiviral vectors were constructed consisting of IL13Rα2 cloned upstream of IRES-eGFP or as a direct fusion to eGFP-RLuc.

FIG. 2D. Western blot analysis revealing the expression of IL13Rα2 and IL13Rα2-eGFP-RLuc in unmodified and LV-tranduced Gli36vIII cells.

FIG. 2E. Cell viability of Gli36vIII-IL13Rα2 GBM cells expressing eGFP-Fluc, co-cultured with hNSC-IL13-PE-mCherry or hNSC-mCherry.

FIG. 2F. Western blot analysis of EGFR expression from the lysates of established human GBM lines.

FIG. 2G. Cell viability of human GBM cells expressing mCherry-Fluc, co-cultured with hNSC-ENb-PE-eGFP or hNSC-eGFP. Scale bars, 100 μm. Data are expressed as mean±s.e.m. Significance of unpaired t test, * P<0.05; # P<0.01; § P<0.001; treated versus control for each GBM line.

FIGS. 3A-3D. IL13-PE decreases GBM viability by blocking protein synthesis and inducing cell cycle arrest

FIG. 3A. Plot of cell viability and protein synthesis in three GBM lines treated with IL13-PE or control conditioned medium and followed daily by simultaneous Fluc and Rluc imaging.

FIG. 3B. Scatter plots of cell cycle analysis performed on U251 GBM cells treated with IL13-PE or control conditioned media. Data are expressed as percentage of total cell population in G1, S, or G2-M.

FIG. 3C. Summary data of cell cycle analysis performed on U251 GBM cells treated with IL13-PE or control conditioned media. Data are expressed as percentage of total cell population in G1, S, or G2-M.

FIG. 3D. U251 GBM cells were engineered to co-express the protein synthesis marker, dsluc, and cell viability marker Rluc. These cells were mixed with either hNSC-Oligo-IL13-PE or unmodified hNSC cells and implanted subcutaneously in SCID mice. Bioluminescence imaging was performed daily to assess protein synthesis and GBM viability. Data are expressed as mean±s.e.m. Significance of unpaired t test, * P<0.05; # P<0.01; § P<0.001; treated versus control for each GBM line.

FIGS. 4A-4D. Stem cell-delivered IL13-PE kills residual tumor and prolongs survival of mice in a GBM resection cavity.

FIG. 4A. Schematic showing how the resection experiment was performed.

FIG. 4B. Quantification of mean Fluc signal intensity obtained of coronal brain section following GBM resections, and plotted before and following surgical resection in both stem cell groups to determine the extent of resection.

FIG. 4C. Plot of Fluc signal intensity before and after tumor resection in treatment groups.

FIG. 4D. Kaplan-Meier survival curves of mice bearing resected U87-Fluc-eGFP tumors in the four treatment groups. Significance of comparison groups assessed by Mantel Cox Log rank test and tabulated. Data are expressed as mean±s.e.m.

FIGS. 5A-5D. IL13-PE has anti-tumor effects on primary human GBMs

FIG. 5A. Semi-quantitative RT-PCR analysis of IL13Rα2 expression from a variety of cancer and stem cell lines.

FIG. 5B. Cell viability of these cancer and stem cell lines following treatment with 25 ng/mL IL13-PE or control conditioned media.

FIG. 5C. hNSCs expressing mCherry or IL13-PE were encapsulated in sECM and co-cultured with primary human GBM cells. Representative photomicrographs of GBM neurospheres and encapsulated hNSCs. Black dashed line indicates edge of sECM.

FIG. 5D. Plot showing GBM cell viability following 5 days culture with encapsulated hNSCs expressing mCherry or IL13-PE. Scale bars, 100 μm. Data are expressed as mean±s.e.m. Significance of unpaired t test, * P<0.05; # P<0.01; § P<0.001; treated versus control for each cell line. Scale bar, 100 μm.

FIGS. 6A-6B. Summary scheme outlining the action of stem cell-delivered PE-cytotoxins in the tumor resection cavity.

FIG. 6A. A cross section of the tumor resection cavity. Residual tumor cells (GBM) are depicted in the resection margins. The cavity has been filled with therapeutic hNSCs encapsulated in sECM. Secreted PE-cytotoxins pass through the sECM matrix where they can act on remaining tumor cells at the resection border.

FIG. 6B. An enlarged projection of the resection cavity highlighting the mechanism of action of the PE-cytotoxin strategy. 1. IL13-PE and ENb-PE cytotoxins are secreted from toxin-resistant hNSCs that are encapsulated in the resection cavity. 2. The PE-cytotoxins bind to their cognate receptor at high affinity. In this case IL13-PE is binding to IL13Rα2 expressed on the cell surface of the GBM cell. 3. Toxin-bound receptor is internalized. Domain II of PE mediates the translocation of the complex into the endosome. 4. Once in the endosome, the protease furin cleaves PE and activates catalytic domain III. 5. The low pH of the endosome compartment causes the toxin to translocate into the cytosol. 6. The catalytic domain traverses the endoplasmic reticulum and inhibits protein synthesis by binding to elongation factor-2. 7. Inhibition of protein synthesis leads to GBM cell death.

FIGS. 7A-7C. Characterizing the hNSC-Oligo cell line.

FIG. 7A. RT-PCR transcript analysis on cDNA derived from unmodified hNSCs and hNSC-Oligo lines. Primer pairs amplify Pax6 and GAPDH products from cDNA.

FIG. 7B. Genomic profiling of parental and modified hNSC lines.

FIG. 7C. Proliferation assay performed on hNSC and bNSC-IL13-PE lines at day 1, 3 and 5. Data are expressed as mean±s.e.m. Scale bar, 100 μm.

FIGS. 8A-8D. Secretion and activity of IL13-PE produced by PE-resistant somatic cells

FIG. 8A. Schematic of vectors encoding the cytotoxins IL13-PE and ENb-PE and the non-toxic variants IL13 and ENb conjugated to either IRES-eGFP or IRES-mCherry.

FIG. 8B. Plot of cell viability of toxin sensitive (293T) and toxin resistant (293DT) cells towards purified PE, and conditioned medium containing IL13 or the toxic IL13-PE variant.

FIG. 8C. Western blot and dot blot (FIG. 8D) analysis using anti-IL13, demonstrating the expression and secretion of IL13-PE from SO-Oligo cells transfected with a vector encoding IL13-PE.

FIGS. 9A-9E. Ectopic expression of IL13Rα2 confers IL13-PE sensitivity to resistant GBMs.

FIG. 9A. Gli36vIII-IL13Rα2-eGFP-RLuc cells were plated at increasing numbers and bioluminescence imaging was performed. The plot reveals a correlation between cell number and Rluc activity.

FIGS. 9B-9E. Gli36vIII cells transduced with LV encoding IL13Rα2 (FIG. 9B) or IL13Rα2-eGFP-Rluc (FIG. 9C) were treated with control or IL13-PE conditioned medium at increasing concentrations (0-750 ng/ml), and cell viability was determined 48 hrs after treatment. Gli36vIII-IL13Rα2 (FIG. 9D) or Gli36vIII-IL13Rα2-eGFP-RLuc (FIG. 9E) cell lines were co-transduced with LV-destabilized-luciferase and treated with 100 ng of IL13-PE or control medium. Levels of protein synthesis were measured by luciferase-based assay 0, 24, 48, or 72 hrs post-treatment.

FIGS. 10A-10C. IL13-PE-expressing somatic cells reduce GBM viability by inhibiting protein synthesis in vivo.

FIG. 10A. Schematic showing how the experiment was performed.

FIGS. 10B-10C. Gli36vIII-IL13Rα2 (FIG. 10B) or U251 (FIG. 10C) GBM cells were co-transduced with eGFP-Rluc (Rluc) and destabilized-luciferase (dsluc), and implanted subcutaneously in a mix with control- or IL13-PE-293DT cells. Dual Fluc and Rluc bioluminescence imaging was performed 0, 1, and 2 days post-implantation to track protein synthesis (PS) and tumor volume (TV) respectively. Representative visible light plus superimposed bioluminescence images of tumors are shown.

FIGS. 11A-11C. Stem cell secretion of ENb-PE reduces GBM viability by inhibiting protein synthesis.

FIG. 11A. GBM cell lines were transduced with LV-destabilized-luciferase (dsluc) and co-cultured with hNSC or hNSC-ENb-PE cells. Aggregate levels of protein synthesis were measured by luciferase-based assay after five days of culture.

FIG. 11B. Schematic showing how the dual bioluminescence in vivo assay was performed.

FIG. 11C. LN229 GBM cells were engineered to co-express the protein synthesis marker, dsluc, and cell viability marker Rluc. These cells were mixed with either hNSC-Oligo-ENb-PE or plain hNSC cells and implanted subcutaneously in SCID mice. Bioluminescence imaging was performed daily and quantified to assess protein synthesis and GBM viability. Representative visible light plus superimposed bioluminescence images of tumors are shown. Data are expressed as mean±s.e.m. Significance of unpaired t test, * P<0.05; # P<0.01; § P<0.001; ENb-PE-treated versus control for each GBM line.

FIGS. 12A-12E. Viability and therapeutic capacity of encapsulated hNSCs.

FIG. 12A. Summary data demonstrating the cell viability of hNSC-mCherry-RLuc cells encapsulated in sECM and cultured for up to five days at different encapsulation numbers.

FIG. 12B. hNSCs or hNSCs secreting IL13-PE were encapsulated in sECM and placed in a culture dish containing U87-eGFP-Fluc. Plot shows tumor cell viability after five days culture as determined by luciferase-based assay.

FIG. 12C. Representative photomicrographs showing encapsulated hNSCs and surrounding U87 GBM cells. Black line demarcates edge of sECM.

FIG. 12D. Plot revealing the strong correlation between U87-eGFP-Fluc cell number and Fluc activity.

FIG. 12E. Representative images and summary data showing retention and viability of sECM-encapsulated hNSC-Fluc cells seeded in the post-resection surgical cavity. Fluc imaging revealing retention of encapsulated hNSCs after 24 hours as seen in the superimposed bioluminescence images. Scale bars, 100 μm. Data are expressed as mean±s.e.m. Significance of unpaired t test, § P<0.001; treated versus control.

FIGS. 13A-13D. Characterizing IL13-PE secretion using a diagnostic variant.

FIG. 13A. Schematic of lentiviral vector encoding a fusion of IL13, Gluc and PE.

FIG. 13B. hNSCs engineered to secrete IL13-Gluc-PE were encapsulated in sECM and cultured for up to seven days. Plot shows secretion of IL13-Gluc-PE as determined by a Gluc-based assay.

FIG. 13C. Concentrated medium containing IL13-GLuc-PE was infused into the borders of the resection cavity and analyzed by serial Gluc imaging.

FIG. 13D. Cells secreting IL13-Gluc-PE, encapsulated in sECM and seeded in the resection cavity were analyzed by serial Gluc imaging. Representative visible light plus superimposed bioluminescence images of mice at various time points.

FIG. 14. GBM modifications do not affect their response to IL13-PE Cell viability of unmodified and transduced (LV-eGFP-Fluc expression) GBMs, following treatment with 25 ng/mL IL13-PE conditioned medium. Data are expressed as mean±s.e.m; ns, not significant.

### DETAILED DESCRIPTION OF THE DISCLOSURE

Unless otherwise explained, all technical and scientific terms used herein have the same meaning as commonly understood by one of ordinary skill in the art to which this disclosure belongs. Definitions of common terms in molecular biology may be found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Further, unless otherwise required by context, singular terms shall include pluralities and plural terms shall include the singular.

Unless otherwise stated, the present disclosure was performed using standard procedures known to one skilled in the art, for example, in Michael R. Green and Joseph Sambrook, Molecular Cloning: A Laboratory Manual, Cold Spring Harbor Laboratory Press, Cold Spring Harbor, N.Y., USA (2012); Sambrook et al., Molecular Cloning: A Laboratory Manual (4th ed.); Davis et al., Basic Methods in Molecular Biology, Elsevier Science Publishing, Inc., New York, USA (1986); Current Protocols in Molecular Biology (CPMB) (Fred M. Ausubel, et al. ed., John Wiley and Sons, Inc.), Current Protocols in Cell Biology (CPCB) (Juan S. Bonifacino et. al. ed., John Wiley and Sons, Inc.), Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998), Methods in Molecular biology, Vol. 180, and Methods in Meolcular Biology, Vol. 203, 2003, which are all herein incorporated by reference in their entireties.

It should be understood that the embodiments of this disclosure is not limited to the particular methodology, protocols, and reagents, etc., described herein and as such may vary. The terminology used herein is for the purpose of describing particular embodiments only, and is not intended to limit the scope of the present disclosure, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages will mean±1%.

All patents and publications identified are expressly incorporated herein by reference for the purpose of describing and disclosing, for example, the methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The Phase III PRECISE trial evaluating Interleukin 13-Pseudomonas exotoxin (IL13-PE) on recurrent glioblastoma multiforme (GBM), failed to meet its efficacy endpoint. Effective clinical translation was confounded by off-target delivery, systemic toxicity and short chemotherapeutic half-life. Embodiments of the present disclosure are based on a new approach that would circumvent the short half-life of PE-fusion proteins in circulation in a subject, thereby providing improved reagents and methods of targeted treatment of cancer using PE. Instead of administering purified PE-fusion proteins such as IL13-PE and ENb-PE systematically into a subject, the new approach is to administer engineered cells that are resistant to the cytotoxic effects of PE and at the same time express and secrete PE-fusion proteins into the subject. This approach ensures an in vivo sustained amount of PE-fusion proteins for targeted cancer therapy as long as the engineered cells remain viable in the subject. The advantage of this approach is that this approach overcomes current clinical limitations by prolonging delivery time and eliminates the requirement for multiple invasive administrations.

Pseudomonas exotoxin (PE) is a single, multi-domain peptide with the ability to enter cells and kill them by catalyzing the inactivation of elongation factor-2 (EF-2), thereby blocking protein synthesis (1). A multitude of antibody variable fragments (Fv) and ligands directed against cancerous cells have been fused to PE (2) to target at cancer cells. Many human cancers, including >50% of glioblastoma multiforme (GBM), express a variant form of the IL-13 receptor called IL-13Rα2, permitting high affinity binding of IL13-PE (3-7). Normal brain cells do not express IL-13Rα2 (8, 9), thus providing a rationale to selectively target and kill GBM cells. Epidermal growth factor receptor (EGFR) is also overexpressed and mutated in a variety of tumors, including GBM, and much effort has been channeled into developing PE-conjugated fusion proteins that target EGFR on malignant cells (10-13).

In the clinic, PE-based cytotoxins have been used with great success to treat a variety of hematologic malignancies including leukemia and Hodgkin's lymphoma (14-18). Yet, attaining similar results in solid tumors has been hindered by inadequate distribution of the cytotoxin throughout the tumor mass coupled to the relatively short half-life of PE. Preclinical testing demonstrated that IL13-PE was highly toxic in culture and in vivo towards IL-13Rα2-expressing cells (7, 19-21), and early phase clinical trials reported that despite some adverse effects, IL13-PE was well tolerated and appeared to have a favorable risk-benefit profile (6, 21). However, in spite of great expectations, the Phase III PRECISE clinical trial failed to show a significant survival benefit in patients with recurrent GBM (22, 23). The failure of this study was likely due to the short half-life of IL13-PE coupled to ineffective delivery of the toxin to residual GBM cells following surgical resection (22).

To overcome these limitations the inventors have engineered toxin-resistant human somatic cells and human neural stem cells (hNSCs) to robustly secrete two PE-cytotoxins, IL13-PE and ENb-PE, that target IL13Rα2 or EGFR respectively, expressed by many GBM (3-6, 24). Nanobodies specific to EGFR (ENb) or mutant EGFR variant (EGFRvIII), have recently been developed that are significantly smaller than conventional antibodies, enabling greater tissue dispersion (25) and the ability to be conjugated to other functional moieties, such as PE (26, 27). The inventors explored the interaction and dynamics of these therapeutic hNSCs in culture and in vivo in multiple models of malignant GBM. Furthermore, the inventors tested the efficacy of IL13-PE-secreting hNSCs in a clinically relevant mouse resection model that were recently developed (28). Cells were encapsulated in a biodegradable synthetic extracellular matrix (sECM) and placed in a resection cavity made by surgically debulking the tumor mass to recapitulate the clinical scenario. The advantage of this cell-based delivery of PE-cytotoxins is that the approach overcomes current clinical limitations by prolonging delivery time and eliminates the requirement for multiple invasive administrations.

Accordingly, in one embodiment, this disclosure provides an engineered mammalian cell that is resistant to PE and also expresses and secretes PE or a protein comprising a PE.

In one embodiment, this disclosure provides an engineered mammalian cell that is resistant to PE and also expresses and secretes PE or a protein comprising PE, for use in the treatment of cancer.

In one embodiment, this disclosure provides an engineered mammalian cell that is resistant to PE and also expresses and secretes PE for use in the manufacture of medicament for the treatment of cancer.

In one embodiment, this disclosure provides a composition comprising of engineered mammalian cells that are resistant to PE and also express and secrete PE or a protein comprising PE. In one embodiment, the composition further comprises a pharmaceutically acceptable carrier. In another embodiment, the composition further comprises an extracellular matrix or a scaffold material.

In one embodiment, this disclosure provides a composition comprising of engineered mammalian cells that are resistant to PE and also express and secrete PE or a protein comprising PE, for use in the treatment of cancer.

In one embodiment, this disclosure provides a composition comprising of engineered mammalian cells that are resistant to PE and also express and secrete PE for use in the manufacture of medicament for the treatment of cancer.

These engineered cells described herein are specially designed to be resistant to PE, i.e., designed not to undergo cell death in the presence of PE, and are also engineered to express and secrete a protein comprising PE. In one embodiment, the cells do not undergo cell death in the presence of a ADP-ribosylating toxin. With this design, the engineered cell is not susceptible to cell death induced by the protein comprising PE that the same engineered cell itself produces and secretes to the surrounding cellular environment. This engineered cell becomes a self-sustaining, cell-based factory for the production of a protein comprising PE in vitro under cell culture conditions and also in vivo when the engineered cells are implanted into a subject.

Another ADP-ribosylating toxin known in the art is diphtheria toxin (DT). Cells that are engineered to be resistant to the cytotoxic effects of PE are also resistant to the cytotoxic effects of DT. Therefore, when these cells are engineered by molecular cloning to express a protein comprising DT, these cells are also not susceptible to cell death induced by the protein comprising DT that the same engineered cell itself produces and secretes to the surrounding cellular environment.

Accordingly, in another embodiment, this disclosure also provides an engineered mammalian cell that is resistant to PE/DT and also expresses and secretes DT or a protein comprising a DT or a fragment, such as a fusion DT protein.

In one embodiment, this disclosure also provides an engineered mammalian cell that is resistant to PE/DT and also expresses and secretes DT or a protein comprising DT or a fragment thereof, for use in the treatment of cancer.

In one embodiment, this disclosure provides an engineered mammalian cell that is resistant to PE/DT and also expresses and secretes DT for use in the manufacture of medicament for the treatment of cancer.

In one embodiment, this disclosure provides a composition comprising of engineered mammalian cells that are resistant to PE/DT and also express and secrete DT or a protein comprising DT or a fragment thereof. In one embodiment, the composition further comprises a pharmaceutically acceptable carrier. In another embodiment, the composition further comprises an extracellular matrix or a scaffold material.

In one embodiment, this disclosure provides a composition comprising of engineered mammalian cells that are resistant to PE/DT and also express and secrete DT or a protein comprising PE, for use in the treatment of cancer.

In one embodiment, this disclosure provides a composition comprising of engineered mammalian cells that are resistant to PE/DT and also express and secrete DT for use in the manufacture of medicament for the treatment of cancer.

In one embodiment, this disclosure provides a method of making an engineered mammalian cell that is resistant to PE or a protein comprising PE, the method comprising: (a) contacting a mammalian cell with an nucleic acid sequence that confers a mutation in a coding sequence of an EF-2 protein; (b) culturing the mammalian cell of step (a) in the presence of a ADP-ribosylating toxin for a period of time; and (c) selecting for the mammalian cell of step (b) that has formed a single colony in the presence of the ADP-ribosylating toxin.

In one embodiment, this disclosure provides a method of making an engineered mammalian cell that is resistant to PE or a protein comprising PE or a DT or an ADP-ribosylating toxin, the method comprising: (a) contacting a mammalian cell with an nucleic acid sequence that confers a G→A mutation in the first nucleotide of codon 717 of a coding sequence of an EF-2 protein; (b) culturing the mammalian cell of step (a) in the presence of a ADP-ribosylating toxin for a period of time; and (c) selecting for the mammalian cell of step (b) that has formed a single colony in the presence of the ADP-ribosylating toxin.

In one embodiment of any aspects disclosed herein, the method further comprising: (e) contacting the colony of cells of step (c) with a vector comprising an exogenous nucleic acid sequence encoding for a PE; (f) culturing the contacted cell of step (d) for a period of time; and (g) selecting for the contacted cell of step (d) for the expression of PE. In one embodiment, the method further comprising culture expansion of the colony of cells of step (c) prior to the contacting step (d).

In one embodiment of any aspects disclosed herein, the method further comprising: (e) contacting the colony of cells of step (c) with a vector comprising an exogenous nucleic acid sequence encoding for a DT or fragment thereof or an ADP-ribosylating toxin; (f) culturing the contacted cell of step (d) for a period of time; and (g) selecting for the contacted cell of step (d) for the expression of DT or fragment thereof or an ADP-ribosylating toxin. In one embodiment, the method further comprises culture expansion of the colony of cells of step (c) prior to the contacting step (d).

In one embodiment of any aspects disclosed herein, the method further comprising collecting and harvesting the colony of cells of step (c) prior to the contacting step (d).

In one embodiment of any aspects disclosed herein, the method further comprising culture expansion of the colony of cells of step (c) prior to the contacting step (d).

In one embodiment of any aspects disclosed herein, the method further comprising collecting and harvesting viable engineered cells that express the PE, the PE fusion protein, diphtheria toxin or fragment thereof or the ADP-ribosylating toxin.

In one embodiment, this disclosure provides a method of making a self-renewing engineered mammalian cell that secretes PE, or a protein comprising PE, or a DT or a fusion protein comprising DT, the method comprising: (a) contacting a mammalian cell that is resistant to Pseudonomas exotoxin, or diphtheria toxin or a ADP-ribosylating toxin with a vector comprising an exogenous nucleic acid sequence encoding for a PE or a fusion protein comprising PE or a fragment thereof, or with a vector comprising an exogenous nucleic acid sequence encoding for a DT or a fusion protein comprising DT or a fragment thereof; (b) culturing the contacted cell of step (a) for a period of time; and (c) selecting for the cell of step (b) for the expression of PE/DT or PE/DT-fusion protein.

In one embodiment, this disclosure provides a method of making a self-renewing engineered mammalian cell that secretes PE or a protein comprising PE, the method comprising: (a) providing an engineered mammalian cell that is resistant to PE; (b) contacting the PE-resistant cell with a vector comprising an exogenous nucleic acid sequence encoding for a PE; (c) culturing the contacted cell of step (b) for a period of time; and (d) selecting for the cell of step (c) for the expression of PE or a protein comprising PE. In one embodiment, the engineered mammalian cell that secretes the expressed PE or a protein comprises PE.

In one embodiment, this disclosure provides a method of making a self-renewing engineered mammalian cell that secretes DT or a protein comprising DT, the method comprising: (a) providing an engineered mammalian cell that is resistant to PE/DT or an ADP-ribosylating toxin; (b) contacting the resistant cell with a vector comprising an exogenous nucleic acid sequence encoding for a DT or a fragment thereof or a protein comprising DT; (c) culturing the contacted cell of step (b) for a period of time; and (d) selecting for the cell of step (c) for the expression of DT or a protein comprising DT. In one embodiment, the engineered mammalian cell that secretes the expressed DT or a protein comprises DT.

In one embodiment, this disclosure provides a method of treating cancer in a subject comprising administering an effective amount of engineered mammalian cells described herein or a composition comprising the engineered mammalian cells described herein into the subject.

In one embodiment, this disclosure provides a method of inducing apoptosis in a tumor or cancer cells in a subject comprising: (a) re-sectioning the tumor from the subject; and (b) implanting a population of engineered mammalian cells described herein or a composition comprising the engineered mammalian cells described herein into the resection site produced in step (a).

In one embodiment, this disclosure provides a method for studying the effectiveness of an engineered cell expressing a cytotoxin, such as a ADP-ribosylating toxin, the method comprising: (a) providing an animal model comprising a cancer in vivo; (b) implanting a population of engineered mammalian cells described herein or a composition comprising the engineered mammalian cells described herein into the animal model; and (c) monitoring the growth of the cancer in the animal model.

In one embodiment, this disclosure provides a method of sustained in vivo delivery of PE or a protein comprising PE or DT or a protein comprising DT in a subject comprising administering an effective amount of engineered mammalian cells of described herein or a composition comprising the engineered mammalian cells described herein into the subject.

The Pseudomonas exotoxin (PE or exotoxin A) is an exotoxin produced by Pseudomonas aeruginosa. It inhibits the protein, elongation factor-2, (EF-2) that is essential for protein synthesis. PE inhibits EF-2 by ADP-ribosylation of a specific histone residue in EF-2. This then causes the elongation of polypeptides to cease, thereby triggering cell death. The mechanism of this toxin is similar to that of Diphtheria toxin (DT). The full length PE protein is described in UniProKB-P11439 (TOXA_PSEAE) and it has 638 amino acid residues. This full length PE includes the signal peptide located at amino acid residues 1-25 and the exotoxin A at located amino acid residues 26-638. (SEQ. ID. NO: 21). The exotoxin A is divided into several domains with each domain having a specific function: domain IA required for target cell recognition (amino acid residues 26-277), domain II is required for translocation in the target cell cytoplasm (amino acid residues 278-389), domain III is required for the ADP-ribosylation activity (amino acid residues 430-638).

Diphtheria toxin or DT is an exotoxin secreted by Corynebacterium diphtheriae, the pathogen bacterium that causes diphtheria. Unusually, the toxin gene is encoded by a bacteriophage, a virus that infects bacteria. The toxin causes the disease diphtheria in humans by gaining entry into the cell cytoplasm and inhibiting protein synthesis. The full length DT toxin protein is described in UniProtKB—P00588 (DTX_CORBE) and it has 567 amino acid residues. This full length diphtheria toxin includes the signal peptide located at amino acid residues 1-32, the fragment A toxin located at amino acid residues 33-225, and the fragment B toxin located amino acid residues 226-567. Once the signal peptide is removed upon secretion, DT is a single polypeptide chain of 535 amino acids consisting of two subunits linked by disulfide bridges, known as an A-B toxin. Binding to the cell surface of the B subunit (the less stable of the two subunits) allows the A subunit (the more stable part of the protein) to penetrate the host cell. The A subunit toxin is enzymatically active, capable of ADP-ribosylation, in the absence of the B subunit toxin.

In one embodiment of any aspects disclosed herein, the engineered mammalian cell that is resistant to PE comprises a modified or mutant EF-2. In one embodiment, the modified EF-2 has a point mutation in the EF-2 protein sequence. In some embodiments, it is the point mutation in the EF-2 protein sequence that results in the modified EP-2 protein being resistant to ADP-ribosylation by PE. Several point mutations comprising single amino acid substitution in the EF-2 are known to confer PE resistance or resistance to ADP-ribosylation by PE. For examples, a serine to glycine substitution at position 584, an isoleucine to asparagine substitution at position 714, a glycine to arginine substitution at position 717, and a glycine to aspartic acid substitution at position 719 of the EF-2 from the Chinese hamster ovary (CHO) cells as described in Foley et al. (47). In some embodiment, the point mutation in the modified EF-2 is selected from the group consisting of a serine to glycine substitution at position 584, an isoleucine to asparagine substitution at position 714, a glycine to arginine substitution at position 717, and a glycine to aspartic acid substitution at position 719 corresponding to the EF-2 of CHO cells or equivalent positions in another mammalian EF-2 when aligned with the EF-2 of CHO cells or as disclosed in Foley et al. (47). Note that the serine at position 584, the isoleucine at position 714, the glycine at position 717, and the glycine at position 719 are conserved in the known EF-2 proteins.

In one embodiment of any aspects disclosed herein, the modified EF-2 expressed in the engineered mammalian cell is not ADP-ribosylated in the presence of PE or DT. Methods of assaying for ADP-ribosylation in a cell and on proteins are known in the art. For example, the method as is described in Foley et al. (47) and in Wang et. al. (55) can be used.

In one embodiment of any aspects disclosed herein, the engineered mammalian cell that is resistant to PE continues to synthesize protein in the presence of PE or DT.

In one embodiment of any aspects disclosed herein, the engineered mammalian cell that is resistant to PE or DT comprises a modified or mutant EF-2 that has a serine to glycine substitution.

In one embodiment of any aspects disclosed herein, the engineered mammalian cell that is resistant to PE or DT comprises a modified or mutant EF-2 that has as isoleucine to asparagine substitution.

In one embodiment of any aspects disclosed herein, the engineered mammalian cell that is resistant to PE or DT comprises a modified or mutant EF-2 that has a glycine to arginine substitution.

In one embodiment of any aspects disclosed herein, the engineered mammalian cell that is resistant to PE or DT comprises a modified or mutant EF-2 that has a glycine to aspartic acid substitution.

In one embodiment of any aspects disclosed herein, the engineered mammalian cell that is resistant to PE comprises a modified or mutant EF-2 that has a G→A mutation in the first nucleotide of codon 717 of a protein coding sequence for the EF-2.

In one embodiment of any aspects disclosed herein, the mutant EF-2 has a G→A mutation in the first nucleotide of codon 717 of a protein coding sequence for an EF-2 protein. In one embodiment, the EF-2 protein is a human EF-2 protein.

In one embodiment of any aspects disclosed herein, the contacting nucleic acid sequence that confers a G→A mutation in the first nucleotide of codon 717 of a coding sequence of an EF-2 protein comprises an oligonucleotide that is at least 90% of SEQ. ID. No: 1, (tgtggggatctggccccctctgcggtggatggcgtcggcgtgca).

In one embodiment of any aspects disclosed herein, the contacting nucleic acid sequence that confers a G→A mutation in the first nucleotide of codon 717 of a coding sequence of an EF-2 protein consists essentially of an oligonucleotide SEQ. ID. No: 1.

In one embodiment of any aspects disclosed herein, the contacting nucleic acid sequence that confers a G→A mutation in the first nucleotide of codon 717 of a coding sequence of an EF-2 protein consists of an oligonucleotide SEQ. ID. No: 1.

In one embodiment of any aspects disclosed herein, the contacting nucleic acid sequence that confers a G→A mutation in the first nucleotide of codon 717 of a coding sequence of an EF-2 protein is oligonucleotide SEQ. ID. No: 1.

In one embodiment of any aspects disclosed herein, the contacting nucleic acid sequence that confers a point mutation in the coding sequence of an EF-2 protein comprises an oligonucleotide that is about 40 to about 60 nucleotides long and the oligonucleotide is exactly identical to the coding sequence of the non-mutant, wild type EF-2 protein except for a single nucleotide change.

In one embodiment of any aspects disclosed herein, the contacting nucleic acid sequence that confers a G→A mutation in the first nucleotide of codon 717 of a coding sequence of an EF-2 protein comprises an oligonucleotide that is about 40 to about 60 nucleotides long and the oligonucleotide is exactly identical to the coding sequence of non-mutant, wild type EF-2 protein except for a single nucleotide change.

In one embodiment of any aspects disclosed herein, the contacting nucleic acid sequence that confers a point mutation in the EF-2 protein sequence, wherein the point mutation is selected from the group consisting of a serine to glycine substitution at position 584, an isoleucine to asparagine substitution at position 714, a glycine to arginine substitution at position 717, and a glycine to aspartic acid substitution at position 719 corresponding to the EF-2 of CHO cells or equivalent positions in another mammalian EF-2 when aligned with the EF-2 of CHO cells or as disclosed in Foley et al. (47). Note that the serine at position 584, the isoleucine at position 714, the glycine at position 717, and the glycine at position 719 are conserved in the known EF-2 proteins.

In other embodiments of any aspects disclosed herein, the contacting nucleic acid sequence is about 42 to about 60 nucleotides long, about 45 to about 60 nucleotides long, about 47 to about 60 nucleotides long, about 50 to about 60 nucleotides long, about 40 to about 57 nucleotides long, about 40 to about 55 nucleotides long, about 40 to about 53 nucleotides long, about 40 to about 50 nucleotides long, about 42 to about 57 nucleotides long, about 42 to about 55 nucleotides long, about 42 to about 53 nucleotides long, about 42 to about 50 nucleotides long, about 44 to about 60 nucleotides long, about 44 to about 57 nucleotides long, about 44 to about 55 nucleotides long, about 44 to about 53 nucleotides long, about 44 to about 50 nucleotides long, about 45 to about 57 nucleotides long, about 45 to about 55 nucleotides long, about 45 to about 53 nucleotides long, about 45 to about 50 nucleotides long, about 47 to about 57 nucleotides long, about 47 to about 55 nucleotides long, about 47 to about 50 nucleotides long, about 49 to about 60 nucleotides long, about 49 to about 57 nucleotides long, about 49 to about 55 nucleotides long, about 49 to about 53 nucleotides long, about 49 to about 51 nucleotides long, about 50 to about 60 nucleotides long, about 50 to about 59 nucleotides long, about 50 to about 57 nucleotides long, about 50 to about 55 nucleotides long, and about 50 to about 53 nucleotides long, and the oligonucleotide is exactly identical to the coding sequence of non-mutant, wild type EF-2 protein except for a single nucleotide change.

In another embodiment, the oligonucleotide is about 47 nucleotides long and is SEQ. ID. NO: 1.

In one embodiment, the contacting nucleic acid sequence is about 47 nucleotides long and is SEQ. ID. NO: 1.

In other embodiments, the contacting nucleic acid sequence is about 41, about 42, about 43, about 44, about 45, about 46, about 48, about 49, about 50, about 51, about 52, about 53, about 54, about 55, about 56, about 57, about 58, or about 59 nucleotides long and comprises SEQ. ID. NO: 1.

An exemplary of non-mutant, wild type EF-2 is the human EF-2. The protein sequence and the cDNA are found in SEQ. ID. NO: 5 and 6 respectively. The human EF-2 protein is also described in UniProtKB—P13639 (EF2_HUMAN), and in GenBank Accession No: X51466.1.

An exemplary of mutant EF-2 is one having a glycine to arginine substitution mutation at codon 717 resulting from a G→A mutation in the first nucleotide of codon 717 of a coding sequence. The mutant coding nucleic acid sequence for such a mutant EF-2 is found in SEQ. ID. NO: 7.

In one embodiment of any aspects disclosed herein, the engineered mammalian cell expresses a mutant EF-2. In one embodiment, this mutant EF-2 is not is posttranslationally ADP-ribosylated in the presence of PE or DT.

The nucleic acid sequence or oligonucleotide that confers the point mutation in the modified EF-2 can be made by any method known in the art. For example, by synthetic oligonucleotide synthesis, site-directed mutagenesis with designed mutagenesis oligonucleotide or by standard cloning methods known in the art. For example, according to methods as described in the Example section herein.

In one embodiment of any aspects disclosed herein, the engineered mammalian cell comprises an exogenous nucleic acid sequence that encodes for a PE or a protein comprising PE.

In one embodiment of any aspects disclosed herein, the engineered mammalian cell expresses a protein comprising PE. In one embodiment, the expressed protein is a fusion protein comprising PE.

In one embodiment of any aspects disclosed herein, the engineered mammalian cell secretes a protein comprising PE. In one embodiment, the secreted protein is a fusion protein comprising PE.

In one embodiment of any aspects disclosed herein, the expressed PE is a fusion protein.

In one embodiment of any aspects disclosed herein, the engineered mammalian cell comprises an exogenous nucleic acid sequence that encodes for a DT or a protein comprising DT.

In one embodiment of any aspects disclosed herein, the engineered mammalian cell expresses a protein comprising DT. In one embodiment, the expressed protein is a fusion protein comprising DT.

In one embodiment of any aspects disclosed herein, the engineered mammalian cell secretes a protein comprising DT. In one embodiment, the secreted protein is a fusion protein comprising DT.

In one embodiment of any aspects disclosed herein, the expressed DT is a fusion protein.

In one embodiment of any aspects disclosed herein, the fusion protein further comprises interleukin 13 (IL-13). Interleukin 13 is a naturally occurring compound in the body that plays a role in the function of the immune system. In addition, a variety of human cancers express IL-13Rα2, a high affinity receptor for IL-13. These cancers include but are not limited to ovarian, breast, colon-rectal, pancreas, and brain cancers. High-grade astrocytomas, the most prevalent and the deadliest form of brain cancer, including glioblastoma multiforme over-express IL-13 receptors in numbers significantly higher than normal tissue. It has been shown that the over expression of IL-13 receptors drives invasion and metastasis of cancers. The ligand IL-13 can be used as the targeting agent for directing cytotoxic therapeutics primarily and specifically to cancers that are overexpressing IL-13 receptors. By combining the targeting molecule, IL-13, with a cellular toxin. This compound acts as a “smart bomb”, delivering the cytotoxin that is specific to cancer cells by local administration.

In one embodiment of any aspects disclosed herein, the fusion protein further comprises epidermal growth factor (EGF). The epidermal growth factor receptor (EGFR; ErbB-1; HER1 in humans) is the cell-surface receptor for members of the epidermal growth factor family (EGF-family) of extracellular protein ligands. The EGFR is a member of the ErbB family of receptors, a subfamily of four closely related receptor tyrosine kinases: EGFR (ErbB-1), HER2/c-neu (ErbB-2), Her 3 (ErbB-3) and Her 4 (ErbB-4). Mutations affecting EGFR expression or activity could result in cancer. Similar to the over expression of IL-13 receptors in cancers, EGFR overexpression have been associated with a number of cancers, including lung cancer, anal cancers, and glioblastoma multiforme. These somatic mutations involving EGFR lead to its constant activation, which produces uncontrolled cell division. In glioblastoma a more or less specific mutation of EGFR, called EGFRvIII is often observed. Mutations, amplifications or misregulations of EGFR or family members are implicated in about 30% of all epithelial cancers. The ligand EGF can be used as the targeting agent for directing cytotoxic therapeutics primarily and specifically to cancers that are overexpressing EGFR or EGFRvIII.

The cDNA coding sequences of PE, DT, IL-13, and EGF are known in the art. The cloning and expression of PE and a protein comprising PE, including PE with mutations and fusion protein comprising PE can be performed by any method known in the art. For example, according to methods as described in the Example section herein, and described in the International PCT Application Publication No: WO 2014/150179 (PCT/US2014/022499), in U.S. Pat. No. 4,830,962, and in the U.S. Patent Application Publication Nos: US 2014/0094417 and US 20030050447, the contents of which are incorporated herein by reference.

As an exemplary, an exogenous nucleic acid sequence that encodes for a PE or a protein comprising PE or a fragment thereof comprises the protein coding sequence for the ADP-ribosylating activity domain IA, II, IB or III of PE. In some embodiments, this would be the regions of amino acid residues 430-638, 431-638, 432-638, 433-638, and 434-638 of PE. In other embodiments, this would be the regions of amino acid residues 85-201, 26-277, 60-200, 80-205, 70-200 of PE. In one embodiment, the exogenous nucleic acid sequence includes the protein coding sequence of IL-13 or EGFR together with that for PE. In one embodiment, the exogenous nucleic acid sequence comprises SEQ. ID. NOS: 2-4, 8, 17, 18, 19, and 22. The protein coding sequence of IL-13 or EGFR is in-frame with that for PE in order to encode a fusion protein when the nucleic acid sequence is transcribed and translated in vivo by the engineered cell. In one embodiment, the IL-13 or EGFR in located at the N-terminus of PE. In one embodiment, the exogenous nucleic acid sequence also includes the protein coding sequence of a signal peptide in order to direct the translated protein for extracellular export. The protein coding sequence of the signal peptide is in-frame with that IL-13 or EGFR and for PE in order to encode a fusion protein when the nucleic acid sequence is transcribed and translated in vivo by the engineered cell. In one embodiment, the signal peptide in located at the N-terminus of the IL-13 or EGFR. In one embodiment, the signal peptide in located at the N-terminus of PE. In one embodiment, the exogenous nucleic acid sequence encodes a fusion protein comprising a signal peptide followed by IL-13 or EGFR, and then followed by the ADP-ribosylating activity domain III of PE, with the signal peptide, IL-13 or EGFR, and PE arranged in tandem, in frame, from N- to C-terminus of the fusion polypeptide.

As an exemplary, an exogenous nucleic acid sequence that encodes for a DT or a protein comprising DT or a fragment thereof comprises the protein coding sequence for the ADP-ribosylating activity fragment A or subunit A toxin. This would be the regions of amino acid residues 33-255, 35-255, 34-255, and 36-255 of DT. In one embodiment, the exogenous nucleic acid sequence includes the protein coding sequence of IL-13 or EGFR together with that for DT. In one embodiment, the exogenous nucleic acid sequence comprises SEQ. ID. NOS: 17 or 18. The protein coding sequence of IL-13 or EGFR is in-frame with that for DT in order to encode a fusion protein when the nucleic acid sequence is transcribed and translated in vivo by the engineered cell. In one embodiment, the IL-13 or EGFR in located at the N-terminus of DT. In one embodiment, the exogenous nucleic acid sequence also includes the protein coding sequence of a signal peptide in order to direct the translated protein for extracellular export. The protein coding sequence of the signal peptide is in-frame with that IL-13 or EGFR and for DT in order to encode a fusion protein when the nucleic acid sequence is transcribed and translated in vivo by the engineered cell. In one embodiment, the signal peptide in located at the N-terminus of the IL-13 or EGFR. In one embodiment, the signal peptide in located at the N-terminus of DT. In one embodiment, the exogenous nucleic acid sequence encodes a fusion protein comprising a signal peptide followed by IL-13 or EGFR, and then followed by the ADP-ribosylating activity fragment A of DT, with the signal peptide, IL-13 or EGFR, and fragment A arranged in tandem, in frame, from N- to C-terminus of the fusion polypeptide.

In one embodiment, the exogenous nucleic acid sequence that encodes for a PE or a protein comprising PE or a fragment thereof or the exogenous nucleic acid sequence that encodes for a DT or a protein comprising DT or a fragment thereof is in a vector. In one embodiment, the vector is an expression vector for delivery and expression of the exogenous nucleic acid sequence in the mammalian cell. Any conventionally known mammalian expression vectors can be used. For example, the lentiviral vectors used in the Example section. Methods of cloning exogenous nucleic acid sequence into vectors for delivery and expression in cells are known in the art.

In one embodiment of any aspects disclosed herein, the mammalian cell is a somatic cell. A somatic cell or vegetative cell is any cell of the body except sperm and egg cells. Somatic cells are diploid, meaning that they contain two sets of chromosomes, one inherited from each parent.

In one embodiment of any aspects disclosed herein, the mammalian cell is a stem cell. A stem cell is an undifferentiated cell of a multicellular organism that is capable of giving rise to indefinitely more cells of the same type (i.e. self-renewal), and from which certain other kinds of specialized cell arise (i.e., by differentiation). Stem cells are undifferentiated biological cells that can differentiate into specialized cells and can divide (through mitosis) to produce more stem cells. They are found in multicellular organisms. In mammals, there are two broad types of stem cells: embryonic stem cells, which are isolated from the inner cell mass of blastocysts, and adult stem cells, which are found in various tissues. In adult organisms, stem cells and progenitor cells act as a repair system for the body, replenishing adult tissues. In a developing embryo, stem cells can differentiate into all the specialized cells—ectoderm, endoderm and mesoderm (see induced pluripotent stem cells)—but also maintain the normal turnover of regenerative organs, such as blood, skin, or intestinal tissues. The two classic properties of stem cells are: (1) Self-renewal: the ability to go through numerous cycles of cell division while maintaining the undifferentiated state, and (2) Potency: the capacity to differentiate into specialized cell types. In the strictest sense, this requires stem cells to be either totipotent or pluripotent—to be able to give rise to any mature cell type, although multipotent or unipotent progenitor cells are sometimes referred to as stem cells.

In one embodiment of any aspects disclosed herein, the mammalian stem cell is an adult stem cell.

There are three known accessible sources of autologous adult stem cells in humans: (1) bone marrow, which requires extraction by harvesting, that is, drilling into bone (typically the femur or iliac crest); (2) adipose tissue (lipid cells), which requires extraction by liposuction; and (3) Blood, which requires extraction through apheresis, wherein blood is drawn from the donor (similar to a blood donation), and passed through a machine that extracts the stem cells and returns other portions of the blood to the donor.

Stem cells can also be taken from umbilical cord blood just after birth. Of all stem cell types, autologous harvesting involves the least risk. By definition, autologous cells are obtained from one's own body, just as one may bank his or her own blood for elective surgical procedures.

In one embodiment of any aspects disclosed herein, the stem cell is selected from a group consisting of neural stem cells, (NSCs), mesenchymal stem cells (MSCs) derived from bone marrow, adipose tissue, cord blood and dental pulp, induced pluripotent stem cells (iPSCs) and iPSC-derived NSC, T cells and MSCs. In one embodiment, the stem cell is a mammalian cell. In one embodiment, the stem cell is a primate mammalian cell. In one embodiment, the stem cell is a human cell.

NSCs are self-renewing, multipotent cells that generate the main phenotype of the nervous system. Stem cells are characterized by their capability to differentiate into multiple cell types via exogenous stimuli from their environment. The isolation and clonal propagation of such cells are known in the art, for examples, in U.S. Pat. Nos. 5,750,376, 5,851,832, 5,849,553, 5,928,947, 5,968,829, 6,294,346, and 6,497,872, the contents of which are incorporated herein by reference.

MSCs are multipotent stromal cells that can differentiate into a variety of cell types, including: osteoblasts (bone cells), chondrocytes (cartilage cells), myocytes (muscle cells) and adipocytes (fat cells). The International Society for Cellular Therapy (ISCT) has proposed a set of standards to define MSCs. A cell can be classified as an MSC if it shows plastic adherent properties under normal culture conditions and has a fibroblast-like morphology. In fact, some argue that MSCs and fibroblasts are functionally identical. Furthermore, MSCs can undergo osteogenic, adipogenic and chondrogenic differentiation ex-vivo. The cultured MSCs also express on their surface CD73, CD90 and CD105, while lacking the expression of CD11b, CD14, CD19, CD34, CD45, CD79a and HLA-DR surface markers. The isolation, clonal propagation and differentiation of such cells are known in the art, for examples, in U.S. Pat. Nos. 5,591,625, 5,827,740, 5,906,934, 6,010,696, 6,261,549, and 6,387,369, the contents of which are incorporated herein by reference.

Induced pluripotent stem cells (also known as iPS cells or iPSCs) are a type of pluripotent stem cell that can be generated directly from adult somatic cells. The introduction of four specific genes encoding transcription factors could convert adult somatic cells into pluripotent stem cells. In one embodiment of any disclosed method, the iPS cell comprises at least an exogenous copy of a nucleic acid sequence encoding a reprogramming factor selected from the group consisting of genes Oct4 (Pou5f1), Sox2, cMyc, Klf4, Nanog, Lin 28 and Glis1. In some embodiments, combinations of reprogramming factors are used. For example, a combination of four reprogramming factors consisting of Oct4, Sox2, cMyc, and Klf4, or a combination of four reprogramming factors consisting of Oct4, Sox2, Nanog, and Lin 28. Methods of producing iPS cell are known in the art, e.g., U.S. Pat. No. 8,058,065, and U.S. Patent Application Nos: 20110223669, 20120214243, 20130059386, and 20130183759, all of which are incorporated herein by reference in their entirety.

In one embodiment of any aspects disclosed herein, the engineered mammalian cells, somatic or stem cells are autologous to the recipient subject being treated for cancer.

In one embodiment of any aspects disclosed herein, the engineered mammalian cells, somatic or stem cells are non-autologous and are allogenic to the recipient subject being treated for cancer.

In one embodiment of any aspects disclosed herein, the engineered mammalian cells, somatic or stem cells are non-autologous and are xenogeneic to the recipient subject being treated for cancer.

In one embodiment of any aspects disclosed herein, the exogenous nucleic acid sequence encodes a fusion PE protein. For example, a IL-13-PE fusion protein and a ENb-PE fusion protein.

In one embodiment of any aspects disclosed herein, the exogenous nucleic acid sequence that encodes for a PE or a protein comprising PE comprising SEQ. ID. No: 2, 3, 4, 8, 17, 18, 19 or 22.

In one embodiment of any aspects disclosed herein, the exogenous nucleic acid sequence that encodes for a PE or a protein comprising PE or a DT or a protein comprising DT comprises SEQ. ID. No: 8, 17 or 18.

In one embodiment of any aspects disclosed herein, the exogenous nucleic acid sequence encodes a fusion PE or a DT fusion that is destined for extracellular secretion. In one embodiment, the exogenous nucleic acid sequence includes a secretion signal sequence to facilitate simultaneous protein synthesis and secretion. For example, the secretion signal sequence is derived from the human flt3 cDNA: atgacagtgctggcgccagcctggagcccaacaacctatctcctcctgctgctgctgctgagctcgggactcagtggg (SEQ. ID. NO:8). Other secretion signal sequences are known in the art can also be used. For example, see the “Signal Peptide Website: An Information Platform for Signal Sequences and Signal Peptides.” In other embodiments, the signal peptides of PE and DT can be used.

In one embodiment of any aspects disclosed herein, the ADP-ribosylating toxin is DT or PE. In other embodiments, fragments of DT or PE having ADP-ribosylating activity can be used.

In one embodiment of any aspects disclosed herein, the method further comprising selecting a subject who has been diagnosed with cancer. Cancers and symptoms are known. A skilled physician would be able to diagnose and select a subject having cancer.

In one embodiment of any aspects disclosed herein, the method further comprising selecting a subject who has been diagnosed with breast, ovarian, pancreas, skin, colon-rectal or brain cancer.

In one embodiment of any aspects disclosed herein, the method further comprising selecting a subject who has been diagnosed with a cancer that express an interleukin 13 receptor (IL-13R), an interleukin 13 alpha 2 receptor (IL-13Rα2) and/or epidermal growth factor receptor (EGFR) and/or mutant EGFR variant (EGFR vIII).

In one embodiment of any aspects disclosed herein, the method further comprising analysis of the cancer cells from the subject for the expression of interleukin 13 alpha 2 receptor (IL-13Rα2) and/or epidermal growth factor receptor (EGFR).

In one embodiment of any aspects disclosed herein, the cancer cells express moderate to high amount of IL-13Rα2.

In one embodiment of any aspects disclosed herein, the cancer cells express moderate to high amount of EGFR.

In one embodiment of any aspects disclosed herein, the cancer cells express the mutant EGFR variant (EGFR vIII).

Determination and analysis of the IL-13Rα2, IL-13R, EGFR or EGFR vIII can be performed by any method known in the art. For example, as it is described in the Example section, determination is performed by RT-PCR analysis and Western blot analysis.

In one embodiment of any aspects disclosed herein, the engineered mammalian cells that express and secret a fusion ENb-PE are used in the treatment when the cancer expresses moderate to high amount of EGFR or expresses EGFR vIII.

As used herein, a cell “expresses EGFR vIII” when there is detectable EGFR vIII determined by any method known. For example, when the method is Western Blot analysis, detectable EGFR vIII means a visible band or signal that is above that of the background or control by Western Blot analysis.

As used herein, “moderate to high amount of a receptor expressed” means at least 5% higher than the basal level of expression in normal non-cancer cells of the same cell or tissue type. In other embodiments, there is at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least one half fold, at least one fold, at least one and a half fold, at least two fold higher than the basal level of expression in normal non-cancer cells. In another embodiment, “moderate to high amount of a receptor expressed” means about 5% higher than the basal level of expression in normal non-cancer cells of the same cell or tissue type. In other embodiments, there is about 6%, about 7%, about 8%, about 9%, about 10%, about 11%, about 12%, about 13%, about 14%, about 15%, about 16%, about 17%, about 18%, about 19%, about 20%, about one half fold, about one fold, about one and a half fold, about two fold higher than the basal level of expression in normal non-cancer cells.

In one embodiment of any aspects disclosed herein, the engineered mammalian cells that express and secret a fusion IL-13-PE are used for treatment when the cancer expresses moderate to high amount of IL-13Rα2.

In one embodiment of any aspects disclosed herein, the cancer comprises solid tumors. A solid tumor is an abnormal mass of tissue that usually does not contain cysts or liquid areas. Solid cancers include cancers of the brain, ovary, breast, colon, lung, skin and other tissues. Examples of solid tumors are sarcomas, carcinomas, and lymphomas. Sarcomas are tumors in a blood vessel, bone, fat tissue, ligament, lymph vessel, muscle or tendon. Carcinomas are tumors that form in epithelial cells. Epithelial cells are found in the skin, glands and the linings of organs. Those organs include the bladder, ureters and part of the kidneys. Lymphoma is any of a group of blood cell tumors that develop from lymphatic cells.

In one embodiment of any aspects disclosed herein, the cancer is selected from a group consisting of brain tumor, melanoma, breast cancer, and lung cancer. In one embodiment of any aspects disclosed herein, the brain tumor is glioblastoma multiforme (GBM),

In one embodiment of any aspects disclosed herein, the engineered mammalian cells are administered systemically.

In one embodiment of any aspects disclosed herein, the engineered mammalian cells are administered locally, directly to or near the cancer cells or tumor in the subject.

In one embodiment of any aspects disclosed herein, the population of engineered mammalian cells is encapsulated prior to implantation. Cell encapsulation or cell microencapsulation technology involves the immobilization of the desired cells within a polymeric semi-permeable membrane that permits the bidirectional diffusion of molecules such as the influx of oxygen, nutrients, growth factors etc. essential for cell metabolism and the outward diffusion of waste products and therapeutic proteins. At the same time, the semi-permeable nature of the membrane prevents immune cells and antibodies from destroying the encapsulated cells regarding them as foreign invaders. The main motive of cell encapsulation technology is to overcome the existing problem of graft rejection in tissue engineering applications and thus reduce the need for long-term use of immunosuppressive drugs after an organ transplant to control side effects.

In one embodiment of any aspects disclosed herein, the population of engineered mammalian cells is encapsulated in a biocompatible polymer or a scaffold material.

In one embodiment of any aspects disclosed herein, the biocompatible polymer or a scaffold material forms mechanically and chemically stable semi-permeable matrix.

In one embodiment of any aspects disclosed herein, the population of engineered mammalian cells is encapsulated in a biocompatible polymer or a scaffold material and the cells are cross-linked to the encapsulation polymer to stabilize the capsules.

A variety of biocompatible polymer materials are available and known in the art for cell encapsulation. For examples, alginate, modified alginate having amino acid sequence Arg-Gly-Asp (RGD) for conjugation purposes, alginate-polylysine-alginate (APA), collagen, gelatin, chitosan, agarose and cellulose sulfate to name a few. Hydrogels made with alginate, or collagen, or gelation, or chitosan, or agarose, or cellulose sulfate or combinations of are commonly used. Any method can be used to encapsulate the described population of engineered mammalian cells. For examples, the method described in the Example section, in U.S. Pat. Nos. 4,353,888, 4,409,331, 5,334,640, 5,459,054, and 5,612,207, all of which are incorporated herein by reference in their entirety.

In one embodiment of any aspects disclosed herein, the tumor re-section is total or partial. As used herein, a “total tumor re-section” means complete excision or removal of all visible tumor tissue. As used herein, a “partial tumor re-section” means incomplete excision or removal of all visible tumor tissue where there is some visible tumor tissue left behind.

In one embodiment of any aspects disclosed herein, the method further comprising implanting a population of engineered mammalian cells of described herein in the periphery of the re-section site.

In one embodiment of any aspects disclosed herein, the method further comprising implanting an additional cancer therapy in the vicinity of the re-section site. In one embodiment, the additional cancer therapy does not comprise engineered cells that expresses PE or are resistant to PE. For example, temozolomide and carmustine (BCNU)-impregnated biodegradable polymer wafers. In another embodiment, an additional cancer therapy is applied at the vicinity of the re-section site. In one embodiment, the additional cancer therapy is applied at the vicinity of the re-section site is radiation therapy.

In one embodiment of any aspects disclosed herein, the cytotoxin is PE or DT.

In one embodiment of any aspects disclosed herein, the cancer forms solid tumors in the animal.

In one embodiment of any aspects disclosed herein, the treatment method further comprising re-sectioning the solid tumors prior to implanting said cells.

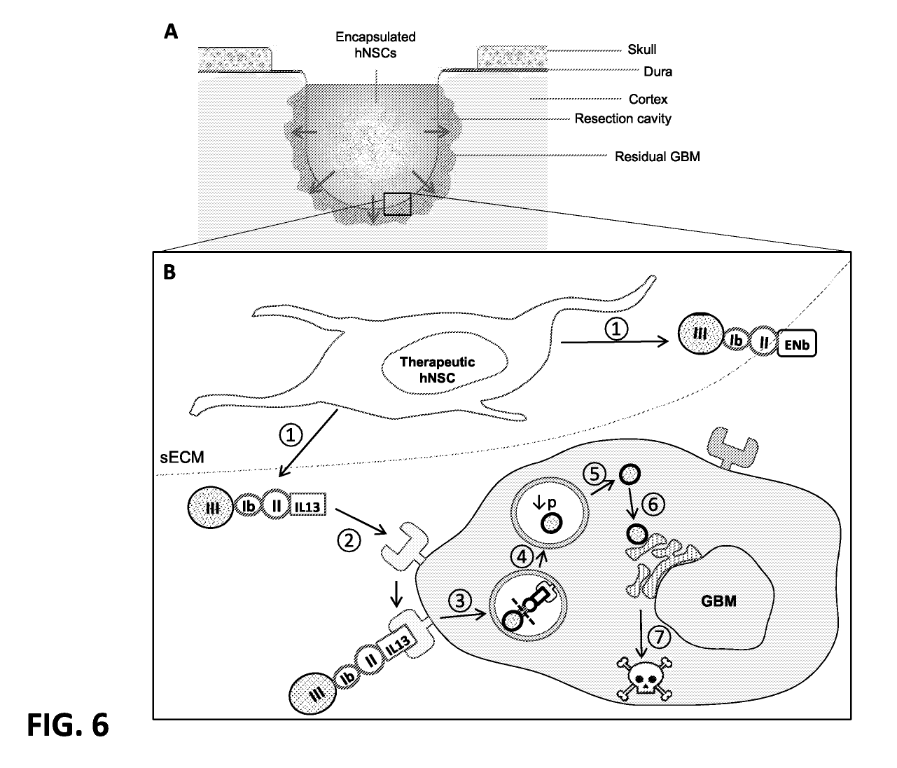

In this study the inventors have engineered toxin-resistant somatic and human neural stem cells (hNSCs) to secrete two PE-cytotoxins, IL13-PE and ENb-PE, that target IL13Rα2 and EGFR respectively, expressed by many GBMs. They showed that both PE-cytotoxins impaired cell viability in multiple GBM lines via protein synthesis inhibition and cell cycle arrest, and that these events could be non-invasively followed in vivo. Furthermore, they showed that IL13-PE-secreting sECM-encapsulated hNSCs transplanted in the surgical resection cavity significantly delayed tumor regrowth and increased survival of mice bearing established GBMs (FIG. 6). Finally they demonstrated efficacy of IL13-PE on patient-derived GBMs and melanoma lines, underscoring its therapeutic relevance and wider therapeutic applicability.

Pioneering work by Pastan and colleagues has helped to propel targeted cytotoxin therapy into the clinical arena, and a multitude of Fv antibody fragments and ligands directed against cancerous cells have been fused to PE and tested in multiple malignancies (2). Despite promising preclinical results, translating PE into humans has been problematic due to a combination of the short half-life of protein formulations and its ineffective delivery throughout the tumor mass. Indeed the Phase III PRECISE clinical trial of IL13-PE on recurrent GBM failed to demonstrate a significant improvement compared to the current standard of care because of these therapeutic limitations (22). It was reported that only 68% of catheters were positioned in accordance with protocol guidelines suggesting that IL13-PE was inadequately distributed to the residual GBM at sufficient concentrations to have a therapeutic effect (22). Recent evidences have shown stem cells can be utilized as unique vehicles for highly effective local delivery of anti-tumor therapies (44-46). In this study the inventors tested that stem cell delivery of PE-cytotoxins could circumvent current limitations by allowing the continuous release of therapeutic agent. Previous studies have demonstrated that a mutant form of elongation factor-2 (EF-2) confers resistance to EF-2-ADP-ribosylating toxins (39, 47), and that mammalian cells can be modified to secrete diphtheria-fused toxin (34). The inventors used a similar strategy to engineer a toxin-resistant stem cell line able to stably secrete PE-cytotoxins by utilizing single-stranded oligonucleotides encoding mutant EF-2 to convert endogenous EF-2 into a toxin-resistant variant. Encapsulation of these therapeutic stem cells in the surgical resection cavity permitted retention and local delivery of therapeutic proteins directly into the resection margins, which could act on residual cancerous cells.

The first stage of treatment for patients suffering from GBM typically consists of surgical debulking of the tumor mass where possible, with substantial resection corresponding to prolonged survival (48, 49). In the vast majority of preclinical GBM studies, therapeutic agents are tested on intact solid tumors. In light of the critical role tumor resection has in GBM management, it was felt that it was essential to use a clinically relevant model to test GBM therapies, in this case one that incorporated debulking of the GBM mass to recapitulate the clinical scenario. Previously a small number of preclinical studies have incorporated surgical resection of GBM (28, 50, 51). The inventors extended this model by incorporating non-invasive bioluminescence imaging (BLI), an approach we have used in previous studies (28, 29, 31). The expression of biomodal (bioluminescent and fluorescent) imaging markers in tumor cells and therapeutic stem cells allowed us to assess multiple processes in vivo including determining the degree of surgical resection, assessing the therapeutic success of IL13-PE by following the growth of residual tumor cells, tracking the kinetics of protein synthesis inhibition and non-invasively confirming the retention of encapsulated hNSCs in the resection cavity. Regarding the last point, BLI indicated that encapsulated hNSCs were efficiently retained in the resection cavity for at least 24 hours.

The inventors have tested the therapeutic efficacy of two PE-cytotoxins, directed against different receptor targets expressed by malignant cells. In the case of IL13-PE, many human cancers, including over half of GBMs, express a variant form of the IL-13 receptor called IL13Rα2, permitting high affinity binding of IL13-PE (3-6). The EGFR pathway is also highly overactive in gliomagenesis, where gene amplification of EGFR and activating mutations in EGFR can be found in up to 70% of all GBMs (24). We show that the therapeutic efficacy of PE-cytotoxins is correlated to the levels of cognate receptor expressed on the GBM cell. We opted to test IL13-PE therapy in the resection model as it showed a somewhat greater efficacy towards the GBM lines we tested compared to ENb-PE. In addition, the expression of IL13Rα2 is largely restricted to malignant cells whereas EGFR is widely expressed by somatic cells (52), thus potentially compromising ENb-PE's cancer-specific mode of action. The choice of U87 GBM in the resection model was three fold: it is a well characterized established GBM line, it expresses moderate levels of IL13RαR2 to represent a more pathophysiological scenario, and it grows as a nodular mass enabling a greater degree of precision during resection, thus facilitating a more tractable resection model.

As an exemplary in practicing the treatment method, in one embodiment, the somatic cells or NSCs are isolated from a host subject. The somatic cells are ex vivo induced in culture into pluripotent stem cells (iPSCs), after which, they are contacted ex vivo with a first nucleic acid sequence which confers a specific mutation to the expressed EF-2 therein. After that or simultaneously, the cells are contacted ex vivo with a second exogenous nucleic acid sequence that encodes a protein comprising PE or DT in order to facilitate the expression of the respective protein comprising PE or DT in the cells. The engineered cells are now resistant to PE/DT by way of a mutant EF-2 that cannot be ADP-ribosylated and are also expressing a protein comprising PE or DT. These engineered cells may be further cultured to expand the cell population (optional), harvested and transplanted (implantation) back into the same host, i.e. an autologous cell transplant.

In one embodiment, the cells are transplanted (implantation) into a subject who is HLA type matched with the donor of the original somatic cells or NSCs prior to contact with any nucleic acid sequences.

In another embodiment, the somatic cells or NSCs are isolated from a donor who is an HLA-type match with a host (recipient) who is diagnosed with cancer. Donor-recipient antigen type-matching is well known in the art. The HLA-types include HLA-A, HLA-B, HLA-C, and HLA-D. These represent the minimum number of cell surface antigen matching required for transplantation. That is the transfected cells are transplanted into a different host, i.e., allogeneic to the recipient host subject. The donor's or subject's somatic cells are ex vivo induced in culture into pluripotent stem cells (iPSCs), after which, they are contacted ex vivo contacted with a first nucleic acid sequence which confers a specific mutation to the expressed EF-2 therein. After that or simultaneously, the cells are contacted with a second exogenous nucleic acid sequence that encodes a protein comprising PE in order to facilitate the expression of a protein comprising PE in the cells. The engineered cells are now resistant to PE by way of a mutant EF-2 that cannot be ADP-ribosylated and are also expressing a protein comprising PE. These engineered cells may be further cultured to expand the cell population (optional), harvested and transplanted (implantation) back into the recipient subject, i.e. non-autologous cell transplant or allogeneic cell transplant.

As another exemplary in practicing the treatment method, in one embodiment, embryonic stem cells (ESCs) are isolated from a host subject, for example, from a fetus in gestation. For example, from the placenta, amniotic fluid, chorionic villi, placental blood and umbilical cord blood of the newborn. The ESCs are induced to differentiate into the neural lineage, thus forming neural progenitor cells. These neural progenitor cells are contacted ex vivo with a first nucleic acid sequence which confers a specific mutation to the expressed EF-2 therein. After that or simultaneously, the cells are contacted ex vivo with a second exogenous nucleic acid sequence that encodes a protein comprising PE in order to facilitate the expression of a protein comprising PE in the cells. The engineered cells are now resistant to PE by way of a mutant EF-2 that cannot be ADP-ribosylated and are also expressing a protein comprising PE. These engineered cells may be further cultured to expand the cell population (optional), harvested and transplanted (implantation) back into the same host, i.e. an autologous cell transplant.

In one embodiment, the cells isolated from a host subject are at the minimum HLA antigen type-matched with a recipient subject having cancer and is being treated for cancer.

In one embodiment of any methods described herein, the engineered cells may culture in 3D in vitro with an extracellular matrix or scaffold material in order to form a 3D tissue. In one embodiment, the engineered cells are culture in 3D in vitro with an extracellular matrix or scaffold material with other cell types to form a tissue. This tissue comprising the engineered cells described herein are then transplanted (implantation) into a subject.

In one embodiment of any methods described herein, the cells are culture ex vivo in sterile conditions, especially when the intended use of the resultant cells is to implant into a subject.

In one embodiment of any methods described herein, the cells are harvested and collected under sterile conditions for the purpose of subsequent implantation into a subject.

Accordingly, provided herein are therapeutic compositions or pharmaceutical compositions that comprised the described engineered mammalian cells that are resistant to PE and also express and secrete a protein comprising PE or DT. Pharmaceutical compositions comprise additionally a pharmaceutically acceptable carrier. In one embodiment, the therapeutic compositions or pharmaceutical compositions are sterile.

The compositions comprising the described engineered mammalian cells that are resistant to PE/DT and also express and secrete a protein comprising PE or DT can be administered by any known route. In some embodiments, the compositions can be formulated for local or systemic delivery (e.g., enteral and parenteral). The compositions comprising the described engineered mammalian cells that are resistant to PE and also express and secrete a protein comprising PE may be administered by any convenient route, for example by infusion or bolus injection and may be administered together with other biologically active agents, agents, such as pharmaceutically acceptable surfactants (e.g., glycerides), excipients (e.g., lactose), carriers, diluents and vehicles. In some embodiments, the compositions can be formulated for delivery to specific organs, for example but not limited to the liver, the spleen, the kidney, and the skin. In one embodiment, the compositions are injected directly into the subject, for example, into the liver, the spleen, the kidney, and the skin.

Routes of administration include, but are not limited to direct injection, intradermal, intramuscular, intraperitoneal, intravenous, subcutaneous, intranasal, epidural, topical, transmucosal, buccal, rectal, vaginal, transdermal, intranasal and parenteral routes. “Parenteral” refers to a route of administration that is generally associated with injection, including but not limited to intraorbital, infusion, intraarterial, intracapsular, intracardiac, intradermal, intrahepatic, intrarogan, intramuscular, intraperitoneal, intrapulmonary, intraspinal, intrasternal, intrathecal, intrauterine, intravenous, subarachnoid, subcapsular, subcutaneous, transmucosal, or transtracheal. Any other therapeutically efficacious route of administration can be used, for example, infusion or bolus injection, absorption through epithelial or mucocutaneous linings.

The dosage of described engineered mammalian cells that are resistant to PE/DT and also express and secrete a protein comprising PE or DT to be administered to a subject will vary depending upon a variety of factors, including the number of described engineered mammalian cells available, the cell encapsulation material, the type and location of the cancer, and the mode and route of administration of the described engineered mammalian cells; size, age, sex, health, body weight and diet of the recipient, the kind of concurrent treatment, frequency of treatment, and the effect desired.

In some embodiments, treatment of a subject with a therapeutically effective dose can include a single treatment (e.g. the transplantation of described engineered mammalian cells that are resistant to PE/DT and also express and secrete a protein comprising PE or DT, or a composition comprising described engineered mammalian cells that are resistant to PE/DT and also express and secrete a protein comprising PE or DT) or a series of treatments.

In one embodiment, the treatment dosage is at least 1×10⁴ described engineered mammalian cells that are resistant to PE and also express and secrete a protein comprising PE per implantation. In other embodiments, the dosage is at least 5×10⁴ cells, at least 1×10⁵ cells, at least 5×10⁵ cells, at least 1×10⁶ cells, at least 5×10⁶ cells, at least 1×10⁷ cells, at least 5×10⁷ cells, at least 1×10⁸ cells, at least 5×10⁸ cells, at least 1×10⁹ cells, at least 5×10⁹ cells, or at least 1×10¹⁰ cells per implantation into a subject.

Second or subsequent administrations can be administered at a treatment dosage which is the same, less than or greater than the initial or previous dose administered to the individual.

A second or subsequent administration may be required during or immediately prior to relapse or a flare-up of symptoms associated cancer regrowth. For example, second and subsequent administrations can be given between about one day to 30 weeks from the previous administration of described engineered mammalian cells that are resistant to PE and also express and secrete a protein comprising PE. Two, three, four or more total administrations can be delivered to the individual, as needed.

The skilled artisan will appreciate that certain factors may influence the dosage and timing required to effectively treat a subject, including but not limited to the cancer type, total or partial re-section, whether the tumor is inoperable, previous treatments, the general health and/or age of the subject, and other diseases present.

The precise dose to be employed in the formulation will also depend on the route of administration, and the cancer type, location etc., and should be decided according to the judgment of the practitioner and each patient's circumstances. Effective doses may be extrapolated from dose-response curves derived from in vitro or animal model test systems.

Efficacy testing can be performed during the course of treatment using the methods described herein. For example, scoring the severity of the various symptoms associated with the cancer type and/or analysis of the level of the cancer markers in the blood of the subject being treated. Measurements of the degree of severity of a number of symptoms associated with the cancer type are noted prior to the start of a treatment and then at later specific time period after the start of the treatment. For example, systematic and periodic monitoring of the level of the cancer markers in the blood of the subject before and after receiving the transplanted described engineered cells. A decrease in the cancer markers levels after transplantation indicates that the treatment is effective in inducing cell death in the cancer cells in the subject.

The present disclosure can be defined in any of the following numbered paragraphs:
[1] An engineered mammalian cell that is resistant to a Pseudonomas exotoxin (PE), and also expresses and secretes PE.
[2] An engineered mammalian cell that is resistant to a PE and also expresses and secretes PE for use in the treatment of cancer.
[3] An engineered mammalian cell that is resistant to a PE and also expresses and secretes PE for use in the manufacture of medicament for the treatment of cancer.
[4] An engineered mammalian cell that is resistant to a PE.
[5] The engineered mammalian cell of any one of paragraphs 1-4, wherein the engineered mammalian cell comprises a modified nucleic acid sequence encoding an elongation factor 2 protein (EF-2).
[6] The engineered mammalian cell of any one of paragraphs 1-5, wherein the engineered mammalian cell expresses a mutant EF-2 protein.
[7] The engineered mammalian cell of paragraph 6, wherein the mutant EF-2 has a G?A mutation in the first nucleotide of codon 717 of the protein coding sequence.
[8] The engineered mammalian cell of any one of paragraphs 1-7, wherein the engineered mammalian cell comprises an exogenous nucleic acid sequence that encodes for a PE.
[9] The engineered mammalian cell of any one of paragraphs 4-8, wherein the engineered mammalian cell expresses a protein comprising PE.
[10] The engineered mammalian cell of any one of paragraphs 4-9, wherein the engineered mammalian cell secretes a protein comprising PE.
[11] The engineered mammalian cell of any one of paragraphs 1-10, wherein the expressed PE is a fusion protein.
[12] The engineered mammalian cell of claim 11, wherein the fusion protein further comprises interleukin 13 (IL-13).
[13] The engineered mammalian cell of claim 11, wherein the fusion protein further comprises epidermal growth factor (EGF).
[14] The engineered mammalian cell of any one of paragraphs 1-13, wherein the mammalian cell is a somatic cell.
[15] The engineered mammalian cell of any one of paragraphs 1-14, wherein the mammalian cell is a stem cell.
[16] The engineered mammalian cell of paragraph 15, wherein the stem cell is selected from a group consisting of neural stem cell (NSC), mesenchymal stem cells (MSCs) derived from bone marrow, adipose tissue, cord blood and dental pulp, and. induced pluripotent stem cell (ipS) and iPS derived NSC, T cells and MSC.
[17] A composition comprising of engineered mammalian cells of any one of paragraphs 1-16.
[18] A composition comprising of engineered mammalian cells of any one of paragraphs 1-16 for use in the treatment of cancer.
[19] A composition comprising of engineered mammalian cells of any one of paragraphs 1-16 for use in the manufacture of medicament for the treatment of cancer.
[20] A composition comprising of engineered mammalian cells that are resistant to a PE and also express and secrete PE.
[21] A composition comprising of engineered mammalian cells that are resistant to a PE and also express and secrete PE for use in the treatment of cancer.
[22] A composition comprising of engineered mammalian cells that are resistant to a PE and also express and secrete PE for use in the manufacture of medicament for the treatment of cancer.
[23] The composition of any one of paragraphs 20-22, wherein the engineered mammalian cell comprises a modified nucleic acid sequence encoding an elongation factor 2 protein (EF-2).
[24] The composition of any one of paragraphs 20-23, wherein the engineered mammalian cell expresses a mutant EF-2 protein.
[25] The composition of paragraph 24, wherein the mutant EF-2 in the engineered mammalian cell of has a G→A mutation in the first nucleotide of codon 717 of the protein coding sequence.
[26] The composition of any one of paragraphs 20-25, wherein the engineered mammalian cell comprises an exogenous nucleic acid sequence that encodes for a PE.
[27] The composition of any one of paragraphs 20-26, wherein the engineered mammalian cell expresses a protein comprising PE.
[28] The composition of any one of paragraphs 20-27, wherein the engineered mammalian cell secretes a protein comprising PE.
[29] The composition of any one of paragraphs 20-28, wherein the expressed PE is a fusion protein.
[30] The composition of paragraph 29, wherein the fusion protein further comprises interleukin 13 (IL-13).
[31] The composition of paragraph 29, wherein the fusion protein further comprises epidermal growth factor (EGF).
[32] The composition of any one of paragraphs 20-31, wherein the mammalian cell is a somatic cell.
[33] The composition of any one of paragraphs 20-31, wherein the mammalian cell is a stem cell.
[34] The composition of paragraph 33, wherein the stem cell is selected from a group consisting of neural stem cell (NSC), mesenchymal stem cells (MSCs) derived from bone marrow, adipose tissue, cord blood and dental pulp, and. induced pluripotent stem cell (ipS) and iPS derived NSC, T cells and MSC.
[35] A method of making an engineered mammalian cell that is resistant to Pseudonomas exotoxin comprising:
   (a) contacting a mammalian cell with an oligonucleotide comprising SEQ. ID. No: 1 that confers a G→A mutation in the first nucleotide of codon 717 of a coding sequence of an elongation factor 2 protein (EF-2);
   (b) culturing the mammalian cell of step (a) in the presence of a ADP-ribosylating toxin for a period of time; and
   (c) selecting for the mammalian cell of step (b) that has formed a single colony in the presence of the ADP-ribosylating toxin.
[36] The method of paragraph 35, the method further comprising:
   (d) contacting the colony of cells of step (c) with a vector comprising an exogenous nucleic acid sequence encoding for a Pseudonomas exotoxin (PE);
   (e) culturing the contacted cell of step (d) for a period of time; and
   (f) selecting for the contacted cell of step (d) for the expression of PE.
[37] The method of paragraph 36, the method further comprising culture expansion of the colony of cells of step (c) prior to the contacting step (d).
[38] The method of paragraph 36 or 37, the exogenous nucleic acid sequence encodes a fusion PE.
[39] The method of paragraph 38, the exogenous nucleic acid sequence encodes a fusion PE that is destined for extracellular secretion.
[40] The method of paragraph 36, 37 or 38, the exogenous nucleic acid sequence comprising sequence selected from the group consisting of SEQ. ID. No: 2-4, 17-18.
[41] The method of any one of paragraphs 35-40, the ADP-ribosylating toxin is diphtheria toxin (DT) or PE.
[42] A method of making a self-renewing engineered mammalian cell that secretes Pseudonomas exotoxin comprising:
   (a) contacting a mammalian cell that is resistant to a Pseudonomas exotoxin (PE) with a vector comprising an exogenous nucleic acid sequence encoding for a Pseudonomas exotoxin (PE);
   (b) culturing the contacted cell of step (a) for a period of time; and
   (c) selecting for the cell of step (b) for the expression of PE.
[43] A method of making a self-renewing engineered mammalian cell that secretes Pseudonomas exotoxin comprising:
   (a) providing an engineered mammalian cell that is resistant to a Pseudonomas exotoxin (PE), a PE-resistant cell;
   (b) contacting the PE-resistant cell with a vector comprising an exogenous nucleic acid sequence encoding for a Pseudonomas exotoxin (PE);
   (c) culturing the contacted cell of step (b) for a period of time; and
   (d) selecting for the cell of step (c) for the expression of PE.
[44] The method of paragraph 41 or 42, wherein the engineered mammalian cell that is resistant to a PE comprises a modified nucleic acid sequence encoding an elongation factor 2 protein (EF-2).
[45] The method of any one of paragraphs 41-44, wherein the engineered mammalian cell that is resistant to a PE expresses a mutant EF-2 protein.
[46] The method of paragraph 45, wherein the mutant EF-2 in the engineered mammalian cell of has a G→A mutation in the first nucleotide of codon 717 of the protein coding sequence.
[47] The method of any one of paragraphs 41-46, the exogenous nucleic acid sequence encodes a fusion PE.
[48] The method of paragraph 47, the exogenous nucleic acid sequence encodes a fusion PE that is destined for extracellular secretion.
[49] The method of any one of paragraphs 41-48, the exogenous nucleic acid sequence comprising sequence selected from the group consisting of SEQ. ID. No: 2-4, 17-18.
[50] A method of treating cancer in a subject comprising administering an effective amount of engineered mammalian cells of any one of paragraphs 1-16 or a composition of any one of paragraphs 17-34 into the subject.
[51] A method of sustained in vivo delivery of Pseudonomas exotoxin in a subject comprising administering an effective amount of engineered mammalian cells of any one of paragraphs 1-16 or a composition of any one of paragraphs 17-34 into the subject.
[52] The method of paragraph 50 or 51, the method further comprising selecting a subject who has been diagnosed with cancer.
[53] The method of paragraph 50 or 52, the method further comprising analysis of the cancer cells from the subject for the expression of interleukin 13 alpha 2 receptor (IL-13R 2) and/or epidermal growth factor receptor (EGFR).
[54] The method of paragraph 53, wherein the cancer cells express moderate to high amount of IL-13R 2.
[55] The method of paragraph 53, wherein the cancer cells express moderate to high amount of EGFR.
[56] The method of paragraph 53, wherein the cancer cell express the mutant EGFR variant (EGFR vIII).
[57] The method of paragraph 50, 52, 55 or 56, wherein the engineered mammalian cells express and secret a fusion EGF-PE when the cancer expresses moderate to high amount of EGFR or expresses EGFR vIII.
[58] The method of paragraph 50, 52 or 54, wherein the engineered mammalian cells express and secret a fusion IL-13-PE when the cancer expresses moderate to high amount of IL-13R 2.
[59] The method of any one of paragraphs 50, 52-58, wherein the cancer comprises solid tumors.
[60] The method of any one of paragraphs 50, 52-59, wherein the cancer is selected from a group consisting of brain tumors-glioblastoma multiforme (GBM), melanoma, breast cancer, and lung cancer.
[61] The method of any one of paragraphs 50-60, wherein the engineered mammalian cells or the composition is administered systemically.
[62] The method of any one of paragraphs 50-60, wherein the engineered mammalian cells or the composition is administered directly to or near the cancer in the subject.
[63] A method of inducing apoptosis in a tumor in a subject comprising:
   (a) resectioning the tumor from the subject; and
   (b) implanting a population of engineered mammalian cells of any one of paragraphs 1-16 or a composition of any one of paragraphs 17-34 into the resection site produced in step (a).
[64] The method of paragraph 63, wherein the population of engineered mammalian cells are encapsulated prior to implantation.
[65] The method of paragraph 63 or 64, wherein the tumor resection is total or partial.
[66] The method of paragraph 63, 64 or 65, further comprising implanting a population of engineered mammalian cells of any one of paragraphs 1-16 or a composition of any one of paragraphs 17-34 in the periphery of the resection site.
[67] The method of any one of paragraphs 63-66, further comprising implanting an additional cancer therapy in the vicinity of the resection site, wherein the additional cancer therapy does not comprise engineered cells that expresses PE or are resistant to PE.
[68] A method for studying the effectiveness of an engineered cell expressing a cytotoxin, the method comprising:
   (a) providing an animal model comprising a cancer in vivo;
   (b) implanting a population of said engineered cells into the animal model; and
   (c) monitoring the growth of the cancer in the animal model.
[69] The method of paragraph 68, wherein the cytotoxin is Pseudonomas exotoxin (PE).
[70] The method of paragraph 68 or 69, wherein the cancer forms solid tumors in the animal.
[71] The method of paragraph 68, 69 or 70, further comprising resectioning the solid tumors prior to implanting said cells.

This disclosure is further illustrated by the following example which should not be construed as limiting. The contents of all references cited throughout this application, as well as the figures and table are incorporated herein by reference.

Those skilled in the art will recognize, or be able to ascertain using not more than routine experimentation, many equivalents to the specific embodiments of the disclosure described herein. Such equivalents are intended to be encompassed by the following claims.

### Example

Materials and Methods

Viral Vector Generation

Recombinant IL13-PE and IL13 were constructed in the previously described Pico2 vector by replacing Firefly luciferase (Fluc) with either IL13-PE or IL13 (29). IL13 was PCR amplified using pORF5-hIL13 (INVITROGEN) as a template with primers encoding Nhe1 and PspX1. The PCR fragment was ligated into Nhe1/PspX1-digested Pico2. To create IL13-PE, IL13 was PCR amplified as described above with primers encoding Nhe1 and EcoV. PE was amplified by PCR with primers encoding EcoV and PspX1 using pJH8 (ATCC) as a template. The two fragments were then ligated into Nhe1/PspX1 digested Pico2. To create ENb-PE, ENb was amplified by PCR as described (26) and ligated into EcoRI/EcoRV-cut pLV-CSC-IG. Additionally, lentiviral vectors (LVs) encoding destabilized luciferase were PCR amplified from pAD-Luc1 (a kind gift from Dr. David Haslam) using primers encoding Nhe1 and Xho1, then ligated into Nhe1/Xho1-digested pLV-CSC-IG that contained an internal ribosomal entry site (IRES) driving eGFP. Construction of LV encoding FLuc-DsRed, GFP-FLuc, and GFP-Rluc have been described previously (29). All LV constructs were packaged as lentiviral vectors in 293T/17 cells using a helper virus-free packaging system as described previously (30). Stem cells and GBM cells were transduced with LVs at varying multiplicity of infection by incubating virions in culture medium containing 4 μg mL⁻¹ protamine sulfate (Sigma) and cells were visualized for fluorescent protein expression by fluorescence microscopy. After expansion in culture, both stem cells and GBM cells were sorted by fluorescence-activated cell sorting (FACSAria Cell Sorting System, BD Biosciences).

Cell Culture

Established human GBM lines U87, LN229, U251, Gli36vIII and primary GBM4, GBM6, GBM23, GBM64 and BT74 cells were grown as described previously (30-33). 293DT cells were cultured as previously described (34). Melanoma and breast cancer cell lines were grown as previously described (35-37). Human neural stem cells (hNSCs) (38), adipose-derived stem cells (hASCs), bone marrow-derived mesenchymal stem cells (hMSCs; kindly provided by D. Prockop, Tulane University) and multipotent cord blood stem cells (hMCBSCs; Cellular Engineering Technologies) were cultured as previously described (29, 39).

Generation of Toxin-Resistant Stem Cell Lines

1. Mutation of endogenous EF-2: A single stranded DNA oligonucleotide (ssODN; IDT, Coralville, Iowa) of 47 bases long was designed to encode the wild-type sequence of EF-2 with a G-to-A transition in the first nucleotide of codon 717 that is known to confer toxin resistance (ssODN-mEF-2) (39). To confirm efficient uptake, a second ssODN was designed that included a 6-carboxyfluorescein (FAM™) on the 3′ end (ssODN-mEF-2-FAM). 293T and hNSCs were transfected with 3 μg of either ssODN-mEF-2 or ssODN-mEF-2-FAM using JET transfection reagent (Polyplus-transfection SA, Illkirch, France) according to manufacturer's specifications. 24 hrs later, media was changed and transfection efficiency was confirmed by fluorescence microscopy on ssODN-mEF-2-FAM cells. Cells were allowed to proliferate for an additional 72 hrs at which time media was refreshed with culture medium containing 20 ng/ml of diphtheria toxin (DT). Cells were cultured under DT selection for 48 hrs, washed and cultured in normal culture medium. Cells were pulsed three additional times for 24 hrs with media containing DT at 20 ng/ml, 50 ng/ml, and 100 ng/ml. Single clones were then expanded and utilized for future experiments.

2. Introduction of IL13-PE and ENb-PE: Toxin-resistant 293T (293-Oligo) or hNSC (hNSC-Oligo) clones were seeded in 6-well plates at a density of 3×10⁵ cells/well. 24 hrs later, cells were transfected with 1.5 μg of LV-IL13-PE or LV-ENb-PE vector (described above), that contained IL13-PE or ENb-PE cloned upstream of a fluorescence marker and puromycin resistance. 24 hrs post-transfection, growth medium was refreshed and transfection efficiency was confirmed by detection of mCherry or eGFP. 48 hrs later, cells were incubated in culture medium containing puromycin (1 μg/mL) for five days. Single clones were selected, expanded, and characterized.

Western Blot Analysis

To investigate the expression of IL13 and IL13-PE, 293DT cells were transfected with IL13 or IL13-PE plasmid DNA and 48 hrs later proteins were isolated from harvested cells, resolved by sodium dodecyl sulfate-polyacrylamide gel electrophoresis (SDS-PAGE), and immunoblotted with antibodies against IL13 (Abcam, Cambridge, Mass.). To determine the expression levels of IL13Rα2 or EGFR in various cancer and stem cell lines, cell lysates were collected, resolved by SDS-PAGE and immunoblotted with an antibody against IL13Rα2 (R&D Systems, Minneapolis, Minn.) or EGFR (BD Biosciences). Blots were developed using enhanced chemiluminescence reagents (Amersham).

RNA extraction and reverse transcription-PCR Analysis

Total RNA was extracted from cells using the RNeasy RNA extraction kit (Qiagen) as per manufacturer's instructions. The optimal RT-PCR conditions for human IL13Rα2 chain amplification have been previously described (40) using the primer pair (sense: 5′-ATGGCTTTCGTTTGCTTGGCTAT-3′, SEQ. ID. NO:9; antisense: 5′-TCATGTATCACAGAAAAATTCTGG-3′, SEQ. ID. NO:10), generating a 1130 base pair (bp) product. A human GAPDH primer pair (sense: 5′-GTCAGTGGTGGACCTGACCT-3′, SEQ. ID. NO:11; antisense: 5′-TGCTGTAGCCAAATTCGTTG-3′, SEQ. ID. NO:12) generated a 245 bp fragment was used as a positive control. A portion of PE was amplified using the primer pair (sense: 5′-GAACCCGACGCACGCGGCCGG-3′, SEQ. ID. NO:13; antisense: 5′-CCGCTCGAGCTTCAGGTCCTCGCGCGGCG-3′, SEQ. ID. NO:14) that generate a 445 bp product. The human PAX6 primer pair (sense: 5′-GAATCAGAGAAGACAGGCCA-3′, SEQ. ID. NO:15; antisense: 5′-GTGTAGGTATCATAACTCCG-3′, SEQ. ID. NO:16) generate a 303 bp product.

Dot Blot Analysis

To determine the expression of IL13 and IL13-PE, 293DT cells were transfected with IL13 or IL13-PE. After 24 hrs of incubation, conditioned medium was collected, spotted on filter paper adjacent to purified IL13 (Chemicon, Billerica, Mass.; 100 ng/μL), and immunoblotted with antibodies against IL13 (Abcam). The blots were quantified with NIH ImageJ and concentrations of IL13-PE were calculated by comparison with purified IL13.

In Vitro Protein Synthesis and Cell Viability Dual Bioluminescence Assays

To investigate the efficacy of PE-cytotoxins, various GBM lines were co-transduced with the reporters LV-Dest-luc (protein synthesis) and LV-Rluc (cell viability) and plated in 96 well plates (Matrical Bioscience). GBM lines were treated with conditioned medium containing known concentrations of PE-cytotoxin. At defined time points, protein synthesis was determined by incubation of cells with 150 μg/mL of D-luciferin (Biotium, Hayward, Calif.) and cell viability was measured by incubation of cells with 1 μg/mL coelanterazine (Nanolight). In non-transduced primary GBM lines, cell viability was determined in separate wells by measuring aggregate metabolic activity using an ATP-dependent luminescent reagent (CellTiter-Glo, Promega, Madison, Wis.). For all in vitro assays, photon emission was measured using a cryogenically cooled high efficiency CCD camera system (Roper Scientific, Trenton, N.J.).

Cell Cycle Analysis

U251 GBM cells were treated with IL13-PE or control conditioned medium. 96 hrs after treatment, cells were pulsed for 1 h with bromodeoxyuridine and propidium iodide (PI)(INVITROGEN) according to manufacturer's instructions. Cells were harvested, stained, and cell cycle progression was processed by FACS and results were analyzed using FlowJo software.

Co-Culture Studies

1. Basic co-culture: To investigate the effect of stem cell-produced IL13 and IL13-PE on GBM cell viability in co-culture analysis, GBM cells (1×10³ cells) transduced with bimodal LV virus were seeded in a 96-well plate (Matrical Bioscience). 24 hrs later, WT or therapeutic stem cells (1×10³ cells) were overlaid on the seeded GBM cells in triplicate. 120 hrs after the addition of stem cells (96 hrs with Gli36vIII co-cultures), fluorescent images were taken and GBM cell viability was determined by Fluc imaging following the addition of 150 μg/mL of D-luciferin (Biotium) to each well.

2. sECM-encapsulated hNSC cell viability and therapeutic efficacy co-culture: The sECM components, Hystem and Extralink (Glycosan Hystem-C, Biotime Inc.), were reconstituted according to manufacturer's instructions. hNSCs were resuspended in Hystem and the matrix was cross-linked by adding half the volume of Extralink. Typically, 4.5 μL drops were placed in the center of glass-bottomed 96-well plates (Matrical Bioscience). After 20 min gelation time the drops were overlaid in triplicate with hNSC media containing GBM cells expressing Fluc, and cultured under standard conditions. GBM cell viability was determined by Fluc imaging as previously described (28, 39). To determine the cell viability of encapsulated hNSCs, hNSCs co-expressing mCherry and RLuc were encapsulated in sECM (500/1000/5000 cells/4.5 L drop) and imaged 1, 3 and 5 days post-encapsulation as previously described (28).

In Vivo Studies

All in vivo procedures were approved by the subcommittee on Research Animal Care at Massachusetts General Hospital.

1. Kinetic Studies: To simultaneously investigate the effects of stem cell-mediated delivery of PE-cytotoxin on GBM cell viability and protein synthesis in vivo, GBM cells were engineered to express Dest-luc (protein synthesis) and Rluc (cell viability) by LV transduction. GBM-Dest-luc-Rluc cells (2×10⁵ cells) were mixed 2:1 with WT/therapeutic hNSC cells (1×10⁵ cells) or unmodified/IL13-PE-transfected 293DT cells and implanted subcutaneously into SCID mice (3 weeks of age, Cox 7, Massachusetts General Hospital, Boston; n>3/group). On days 0, 1, and 2, Rluc imaging was performed by i.p. injection of coelenterazine (CaliperLS, Xenolight; 3.3 μg/g body weight) to determine tumor volume. To determine protein synthesis in the same tumors, Rluc imaging was followed 8 hrs later by Fluc imaging performed by administration of D-luciferin (1 mg/animal in 100 μL saline).

2. Assessing retention of encapsulated hNSCs: To determine the retention of encapsulated hNSCs in the resection cavity, U87-eGFP-FLuc tumor cells (0.5×10⁶ cells) were superficially implanted into SCID mice (n=3) through established cranial windows as previously described (28). Three days later, tumors were resected and 2×10⁶ bNSCs transduced to express mCherry-Fluc were encapsulated in sECM and seeded in the resection cavity. FLuc imaging was performed on days 1 and 3 by intraperitoneal injection of D-Luciferin (1 mg/mouse).

3. Therapeutic Efficacy in GBM resection model: To determine the effects of hNSC-IL13-PE or infusion of IL13-PE protein on GBM progression, U87-GFP-FLuc cells (0.5×10⁶) were implanted into SCID mice through established cranial windows (n=25). 6 days later, mice underwent surgical tumor resection as previously described (28). bNSC-IL13-PE (n=7) or hNSC-mCherry (n=7) cells were encapsulated in sECM (2×10⁶ cells) and seeded in the resection cavity, or 10 μls of concentrated conditioned media (n=5) containing 40 ng IL13-PE was infused into the cavity boarder. There was also a resection alone group (n=4) where the tumor was resected and the cavity left untreated. Tumor progression was monitored by serial Fluc imaging performed between days 1 to 85 following intraperitoneal injection of D-Luciferin (1 mg/mouse).

Tissue Processing

Mice bearing resected tumors were perfused with formalin and brains were extracted. After 24 hours in formalin, brains were transferred to 30% w/v sucrose. 24 hours later, brains were sectioned on a vibratome at a thickness of 20 μm. Photomicrographs and fluorescence images of brain sections were acquired using a Nikon E400 light microscope attached to a SPOT CCD digital camera (Diagnostic Instruments) and processed using ImageJ software.

Statistical Analysis

Data were analyzed by Student t-test when comparing two groups and expressed as mean±s.e.m. Differences were considered significant at P<0.05 (*); P<0.01 (#); P<0.001 (§). Survival times of mouse groups were compared using a Mantel Cox log-rank test.

Results

Construction of Toxin Resistant Stem Cells that Secrete PE-Cytotoxin

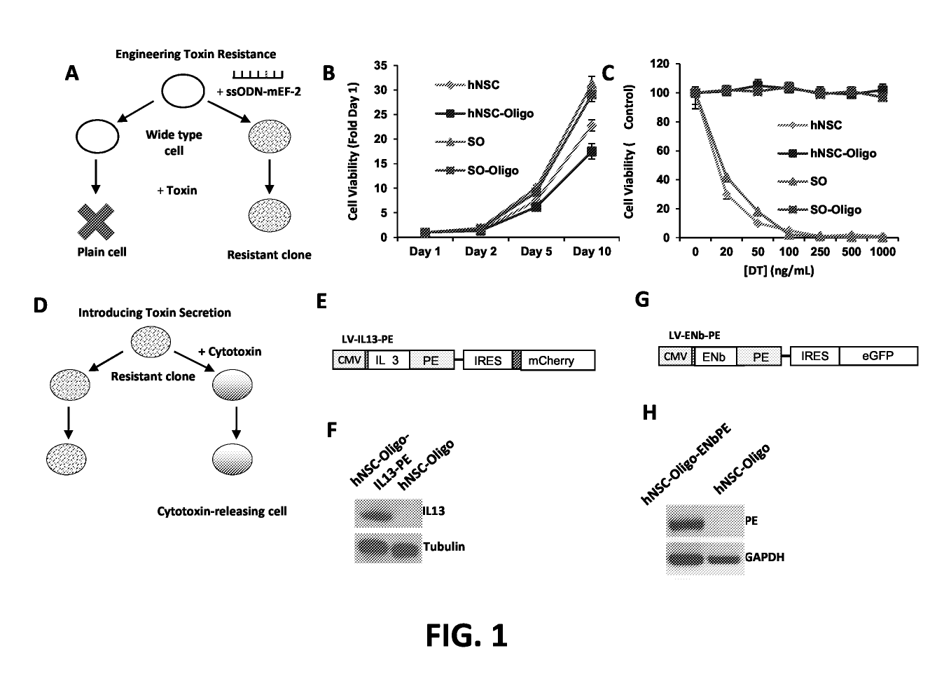

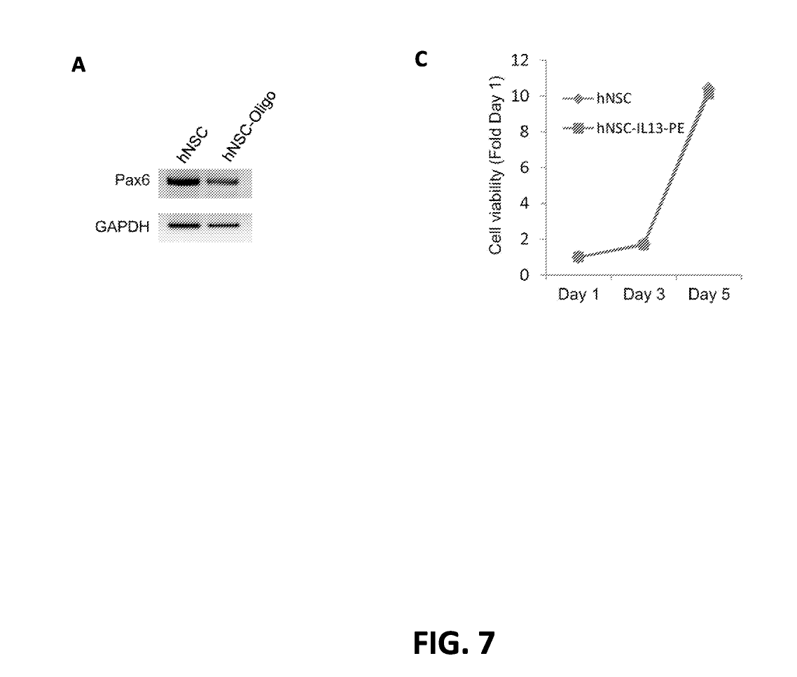

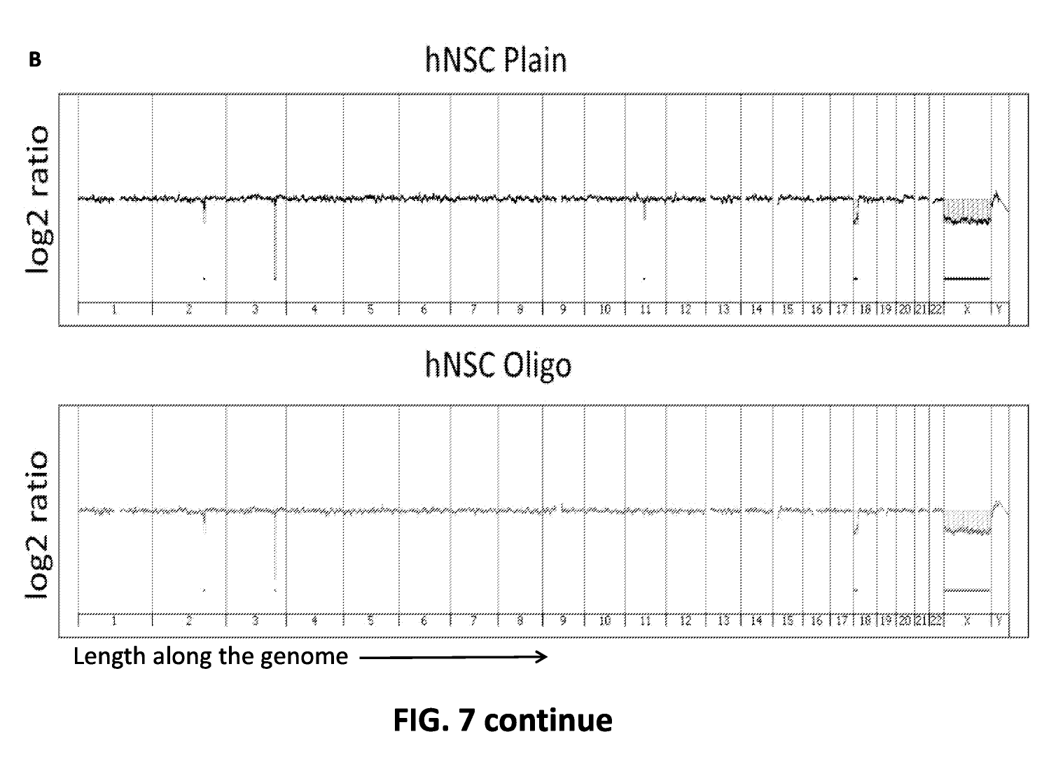

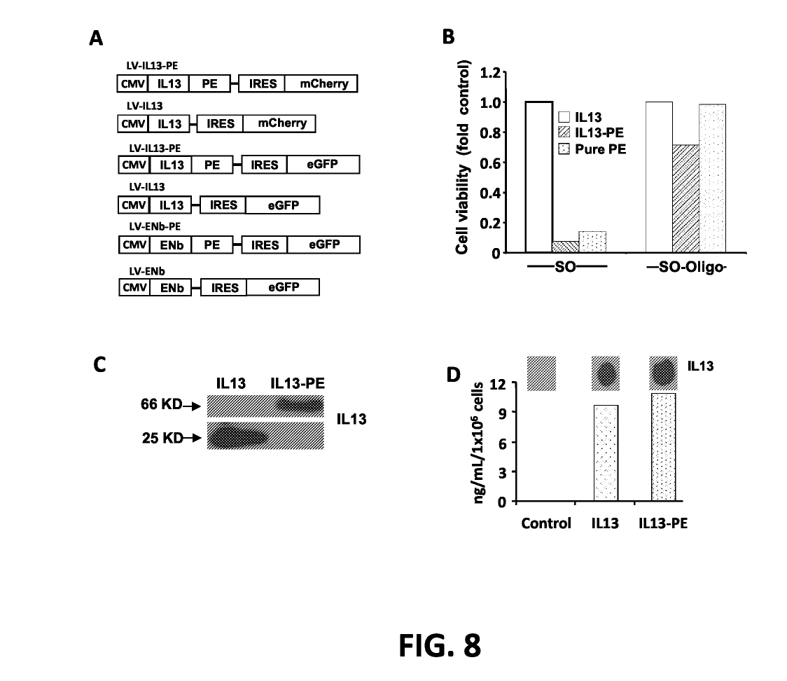

Elongation factor-2 (EF-2) can be specifically inactivated by ADP-ribosylating toxins such as diphtheria toxin (DT) and pseudomonas exotoxin (PE), thereby inhibiting protein synthesis and killing the cell (41-43). To engineer human cell lines capable of delivering PE-based cytotoxins, the inventors first rendered them resistant to PE (FIG. 1A). Toxin resistance was conferred by utilizing single-stranded oligonucleotides that converted endogenous EF-2 into a toxin-resistant variant in both human somatic cells (SO-Oligo) and human neural stem cells (hNSC-Oligo) (FIG. 1A). The mutation of EF-2 had no marked impact on the proliferation rates, or other cellular characteristics of either Oligo cell line (FIGS. 1B and 7). Both SO-Oligo and hNSC-Oligo cell lines displayed resistance to purified DT up to a concentration of 1000 ng/mL (FIG. 1C), an effect not observed in parental lines. A number of plasmids encoding IL13-PE, ENb-PE, and non-PE containing variants were constructed (FIG. 8A) and initially characterized in SO-Oligo cells. Fluorescence images of 293T (SO) or 293-Oligo (SO-Oligo) cells transiently transfected with vectors encoding IL13-PE or IL13. Expression of eGFP was determined 24 hours post-transfection. SO-Oligo cells were able to transiently express IL13-PE, unlike parental lines which were only able to express the non-toxic IL13 version (data not shown). Furthermore, unmodified SO cells displayed a marked reduction in cell viability upon treatment with conditioned medium containing IL13-PE, whilst toxin-resistant SO-Oligo cells were unaffected (FIG. 8B). To quantify the secretory capacity of SO-Oligo cells, they were transfected with IL13-PE or IL13 plasmids (FIG. 8C), and analysis of conditioned medium revealed proteins were secreted at >10 ng/ml/106 cells (FIG. 8D). To create therapeutic stem cell lines that stably secreted PE-cytotoxins, hNSC-Oligo lines were transfected with plasmids encoding PE-cytotoxins upstream of a fluorescent reporter, to enable identification of stably expressing clones (FIG. 1D). hNSC-Oligo cells were engineered to stably express IL13-PE (FIGS. 1E and 1F) and EGFR Nanobody (ENb)-PE (FIGS. 1G and 1H). These results confirm that human somatic and hNSCs can be modified to display resistance to EF-2-ADP-ribosylating toxins and engineered to secrete functional PE-cytotoxins.

Stem Cell-Delivered PE-Cytotoxins Reduce Cell Viability in Multiple GBMs

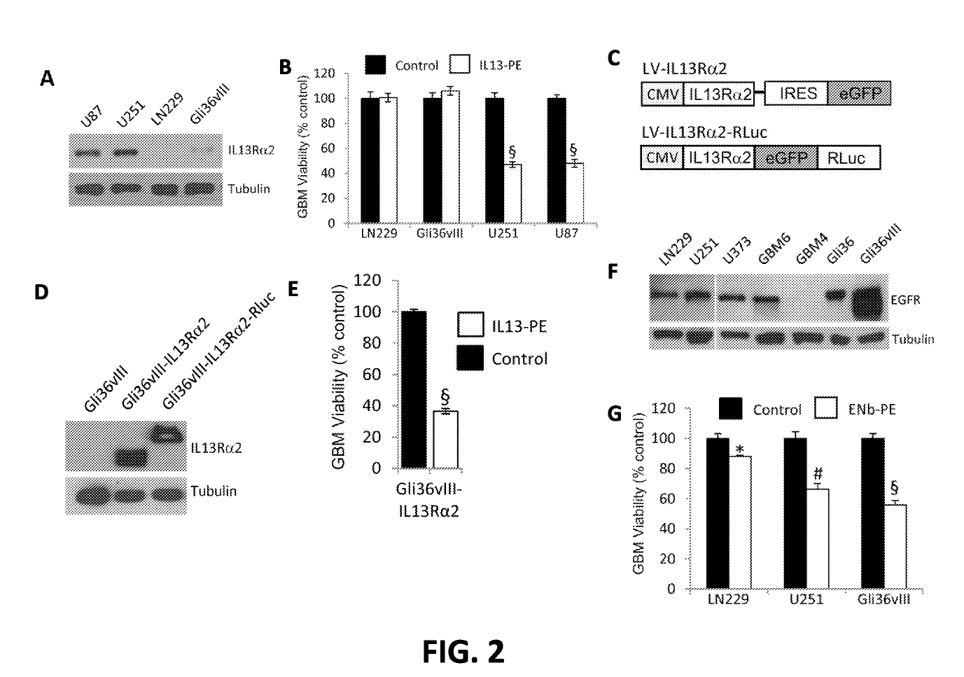

To establish potential sensitivity of GBMs towards cell-delivered IL13-PE, levels of their cognate receptor, IL13Rα2, were determined in multiple established GBM lines (FIG. 2A). Variation in receptor protein levels corresponded to the degree of response to IL13-PE treatment in co-culture experiments (FIG. 2B). Furthermore, ectopic over-expression of IL13Rα2 in a GBM line with low endogenous levels of receptor (Gli36vIII-IL13Rα2 or Gli36vIII-IL13Rα2-RLuc; FIGS. 2C and 2D) displayed the greatest degree of sensitivity to hNSC-IL13-PE, indicating the requirement of cognate receptor for cytotoxin binding (FIG. 2E). To assess the potential sensitivity of GBMs to hNSC-ENb-PE, EGFR expression was analyzed in a panel of GBMs (FIG. 2F). Again, a decrease in GBM viability was correlated to the level of EGFR expressed, with the line expressing constitutively active EGFR (Gli36vIII) showing the greatest efficacy (FIG. 2G). These results demonstrate that stem cell-delivered PE-cytotoxins reduce the viability of GBM lines in a response that is consistent to the level of receptor expressed by the target GBM line.

Imaging the Kinetics of IL13-PE Action on GBMs In Vitro and In Vivo

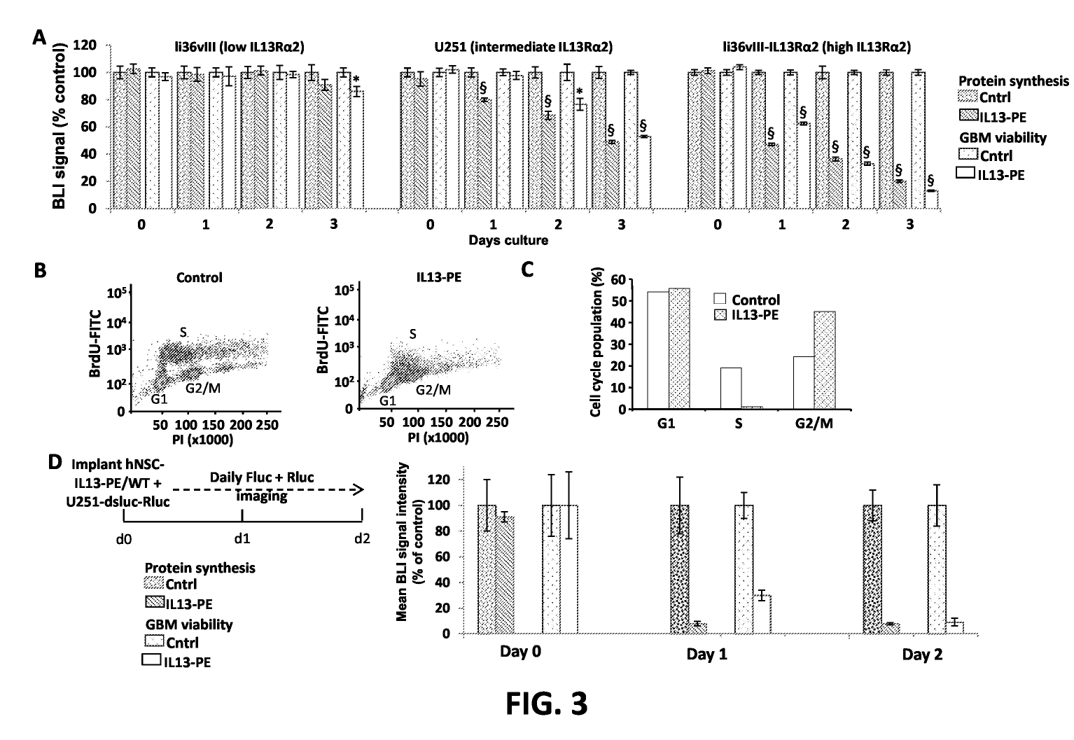

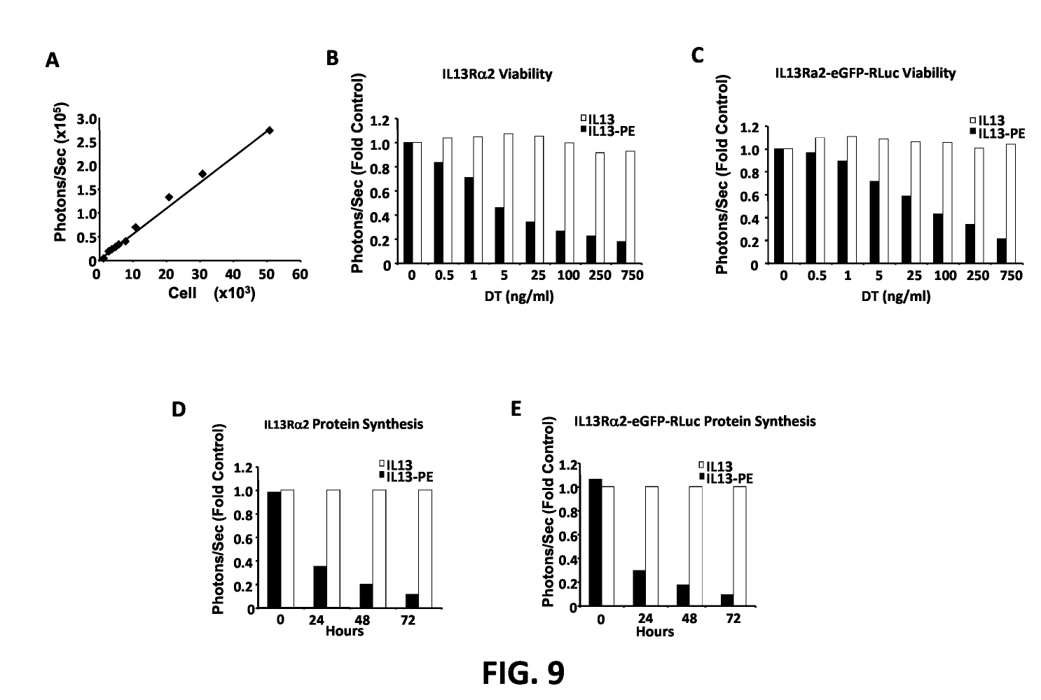

Given the GBM-specific expression of IL13Rα2 versus the widespread distribution of EGFR, coupled to our previous data indicating that stem cell-delivered IL13-PE was more efficacious than ENb-PE in the GBM lines tested, the inventors largely focused on this IL13-PE cytotoxin in subsequent experiments. To investigate the molecular mechanisms that mediate IL13-PE toxicity and to define the kinetics of cytotoxin action, three GBM lines expressing low (Gli36vIII), intermediate (U251) and high (Gli36vIII-IL13Rα2) levels of receptor were transduced to express the novel protein synthesis reporter destabilized luciferase (dsluc), in addition to Renilla luciferase (Rluc) to assess cell viability (FIGS. 3A and 9). Dual bioluminescence imaging (BLI) was performed daily to simultaneously assess the extent of protein synthesis and cell viability in GBMs treated with control or IL13-PE-containing conditioned medium (FIG. 3A). IL13-PE treatment did not inhibit protein synthesis or affect cell viability in Gli36vIII that lack IL13Rα2, indicating the requirement of this receptor for therapeutic efficacy (FIG. 3A). Response to IL13-PE was most rapid and profound in Gli36vIII-IL13Rα2 cells overexpressing the receptor. In addition, inhibition of protein synthesis preceded the reduction in cell viability providing evidence that PE-induced toxicity was via inhibition of protein synthesis (FIG. 3A). Cell cycle analysis revealed that protein synthesis inhibition was associated with a marked reduction in the number of cells in S-phase and an accumulation of cells in G2/M phase (FIGS. 3B and 3C). These results demonstrate that binding of IL13-PE to IL13Rα2 causes inhibition of protein synthesis, induces cell cycle arrest and ultimately reduces GBM cell viability in culture.

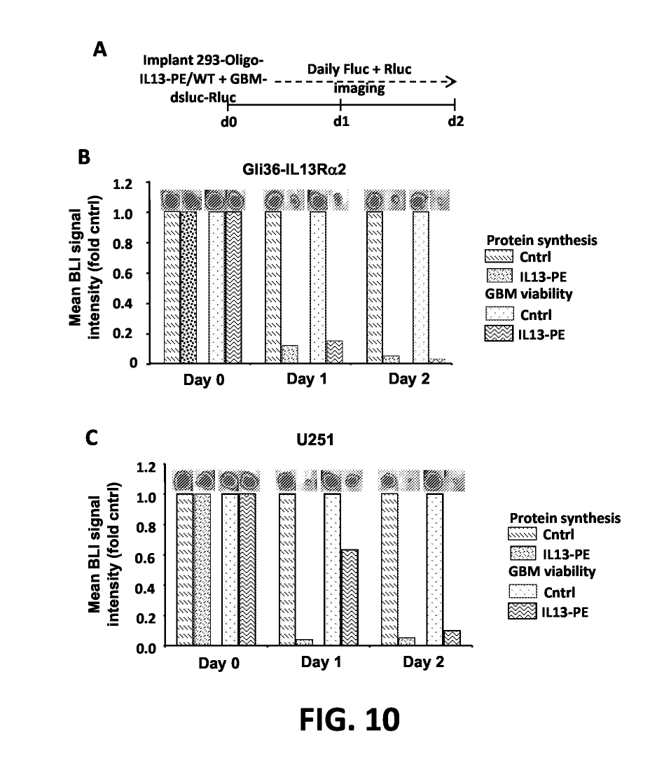

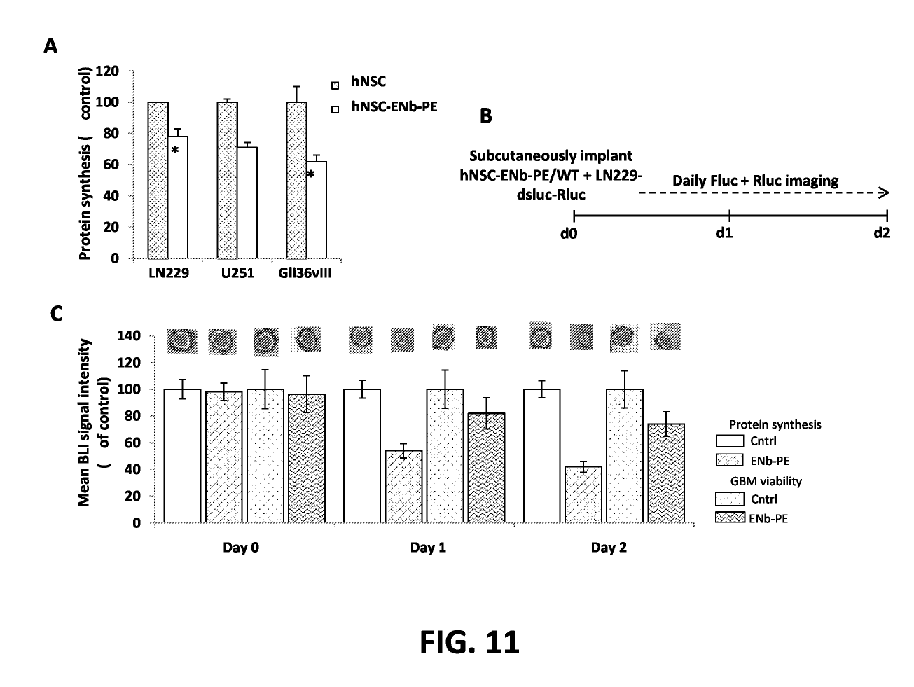

To test if IL13-PE secreted by hNSCs could cause a similar response to GBMs in vivo, the inventors applied non-invasive BLI to track protein synthesis and cell viability in sub-cutaneous tumors made by mixing U251-dsluc-Rluc cells with either hNSC-IL13-PE or unmodified hNSCs (FIG. 3D). Stem cell-secreted IL13-PE reduced protein synthesis in U251 cells by over 90% as early as 24 hours post-treatment that persisted through 48 hours (FIG. 3D). This was accompanied by a 70% reduction in cell viability after 24 hours that increased to >90% by 48 hours (FIG. 3D). Similar results were obtained by using human somatic cells expressing IL13-PE (FIG. 10) and bNSC-ENb-PE (FIG. 11). Together, these results indicate that cellular delivery of PE-cytotoxins can efficiently and robustly reduce GBM viability in vivo by inhibiting protein synthesis, and that these anti-tumor effects can be tracked non-invasively using BLI.

Stem cell-delivered IL13-PE kills residual tumor and prolongs survival of mice in a GBM resection model.

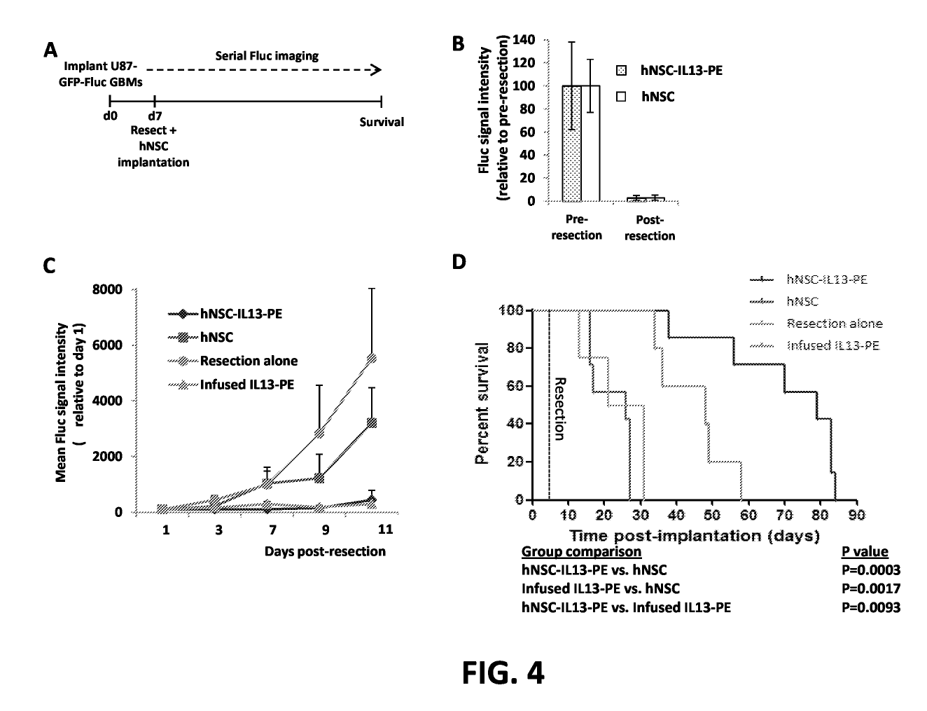

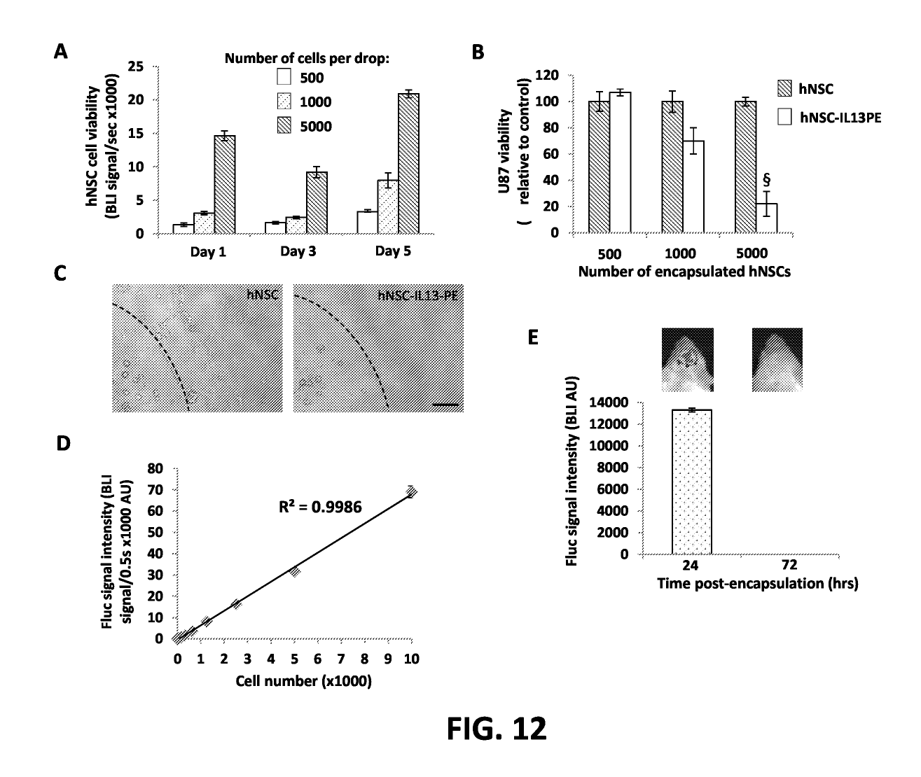

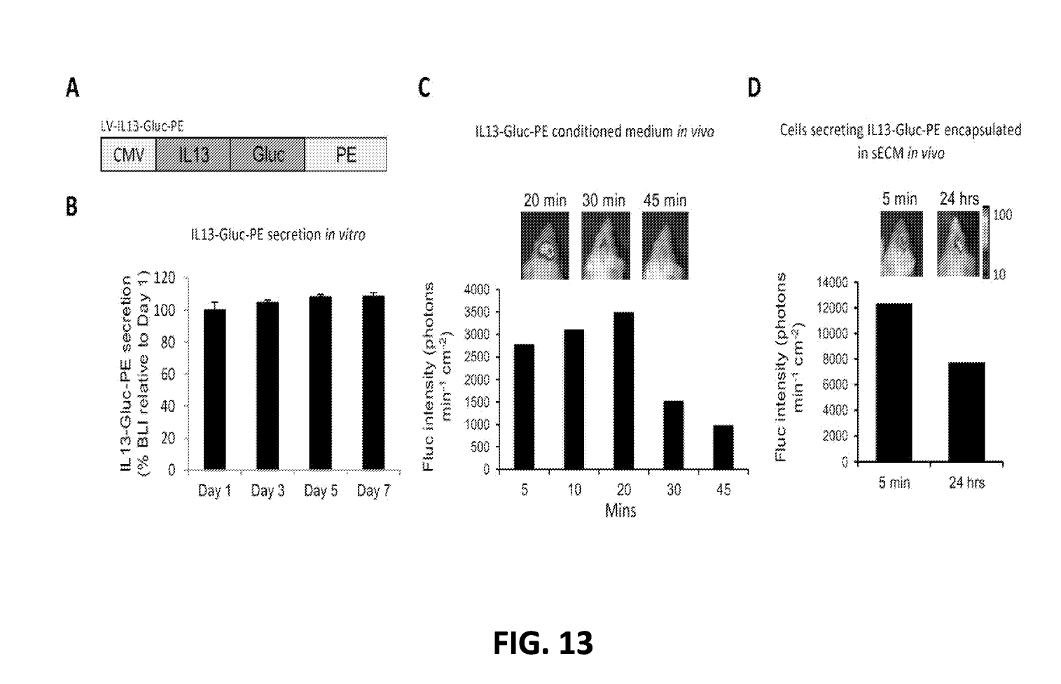

One of the major limitations in current GBM therapies is the inadequate distribution of chemotherapeutic agents towards residual GBM cells following surgical resection. To investigate the efficacy of stem cell-delivered IL13-PE on residual tumor cells in vivo, they were encapsulated in a synthetic extracellular matrix (sECM) and applied to a mouse tumor resection model according to the schedule in shown FIG. 4A. In culture, encapsulated bNSCs remained viable and could escape the sECM (FIG. 12A). Furthermore, U87 GBM cells were shown to be sensitive to IL13-PE secreted by encapsulated hNSC-IL13-PE cells in vitro (FIGS. 12B and 12C), indicating they would be an ideal GBM line to use in this resection experiment. U87-GFP-Fluc tumors were surgically debulked under a fluorescence microscope (data not shown), and encapsulated hNSCs were injected into the resection cavity (data not shown). A cranial window was established in mice and 2×105 U87-Fluc-eGFP cells were superficially implanted in the cranial window. The extent of surgical resection was determined by comparing Fluc signal pre- and post-resection, with >95% of the tumor typically resected (FIGS. 4B and 12D). Mice were followed longitudinally for changes in tumor volume by serial BLI (data not shown). 21 days after resection variable tumor masses had developed in the control sECM-hNSC and IL13-PE infusion groups, whilst no tumor could be detected in the sECM-hNSC-IL13-PE group (data not shown). This was most likely due to the initial retention of encapsulated therapeutic stem cells in the resection cavity, versus the transient exposure of infused IL13-PE (FIGS. 12E and 13). Indeed, this group conferred a statistically significant survival benefit with a median survival of 79 days versus 48 days in the IL13-PE infusion group and 26 days in both the resection alone and encapsulated control hNSC groups (P=0.0003 versus hNSC group; P=0.0093 versus IL13-PE infusion group; FIG. 4D). These results demonstrate that encapsulated hNSCs secreting IL13-PE significantly increase anti-GBM efficacy compared to direct injection of IL13-PE protein in a preclinical model of GBM resection.

IL13-PE has Anti-Tumor Effects in Patient-Derived GBMs

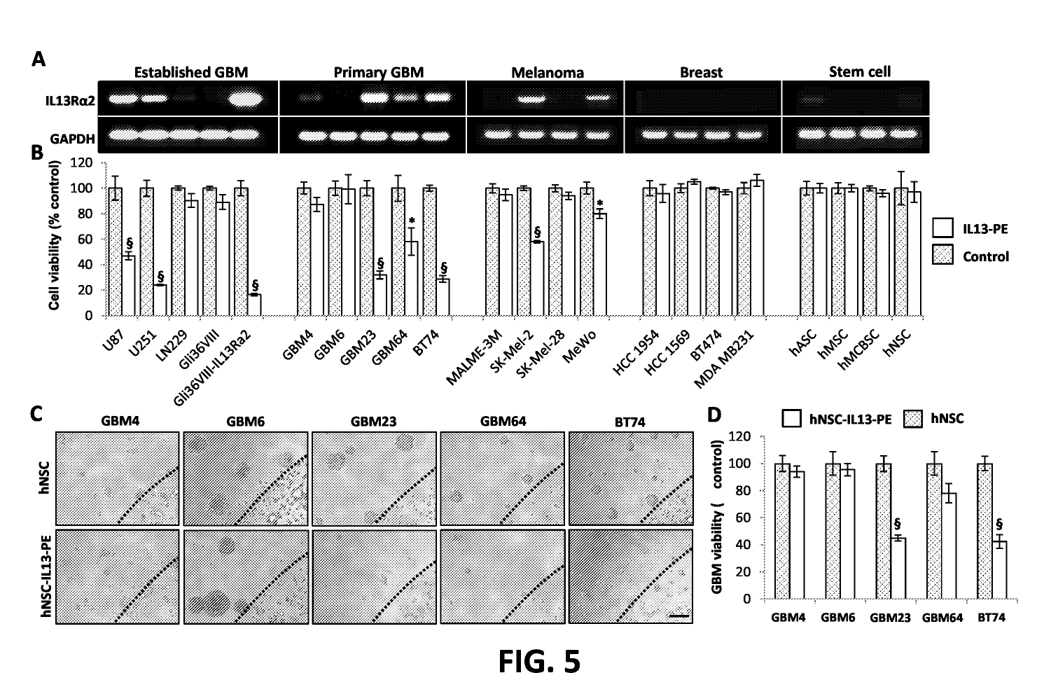

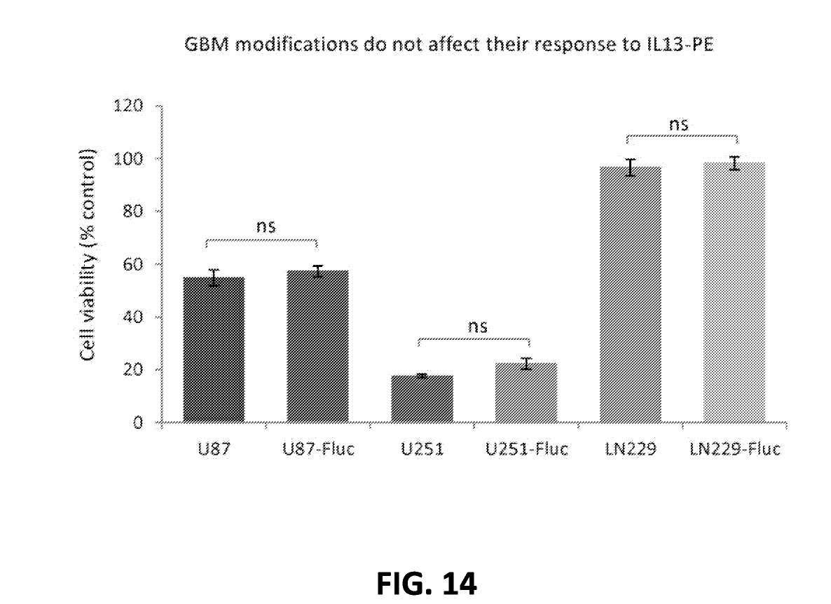

To investigate the clinical potential and wider applicability of IL13-PE as a therapeutic agent, the presence of IL13Rα2 transcript was assessed in five patient-derived GBM lines and a panel of cancer and stem cell lines (FIG. 5A). The efficacy of IL13-PE was once again correlated to IL13Rα2 transcript levels expressed by the cancer cells (FIG. 5B, FIG. 14). Patient-derived GBM lines expressing robust IL13Rα2 transcript (GBM23, GBM64 and BT74) displayed a significant reduction in cell viability upon IL13-PE treatment (FIG. 5B). This correlation was also observed in established GBM and melanoma lines (FIG. 5B). None of the stem cell lines tested responded to IL13-PE treatment confirming the cancer-selective nature of IL13-PE (FIG. 5B). To test if stem cell-delivered IL13-PE could also act on primary patient-derived GBMs, hNSC-IL13-PE or unmodified hNSCs were encapsulated in sECM and surrounded by primary GBMs (FIG. 5C). GBM23 and BT74 displayed a profound decrease in viability compared to controls (P<0.0001; FIG. 5D). These results demonstrate that sECM-encapsulated hNSCs expressing IL13-PE have therapeutic efficacy against primary patient-derived GBMs that express IL13Rα2. Furthermore, IL13-PE can act on non-GBM cancers, indicating broader therapeutic potential.

The references cited herein and throughout the specification are incorporated herein by reference.

### REFERENCES

1. J. Hwang, et al., Functional domains of Pseudomonas exotoxin identified by deletion analysis of the gene expressed in E. coli. Cell 48:129 (1987).
2. J. E. Weldon, and I. Pastan, A guide to taming a toxin-recombinant immunotoxins constructed from Pseudomonas exotoxin A for the treatment of cancer. FEBS J. 278:4683 (2011).
3. H. Okada et al., Expression of glioma-associated antigens in pediatric brain stem and non-brain stem gliomas. J. Neurooncol. 88:245 (2008).
4. J. Wykosky, et al., Interleukin-13 receptor alpha 2, EphA2, and Fos-related antigen 1 as molecular denominators of high-grade astrocytomas and specific targets for combinatorial therapy. Clin. Cancer Res. 14:199 (2008).
5. J. S. Jarboe, et al., Expression of interleukin-13 receptor alpha2 in glioblastoma multiforme: implications for targeted therapies. Cancer Res. 67:7983 (2007).
6. W. Debinski, et al., Expression of a restrictive receptor for interleukin 13 is associated with glial transformation. J. Neurooncol. 48:103 (2000).
7. M. Candolfi et al., Gene therapy-mediated delivery of targeted cytotoxins for glioma therapeutics. Proc. Natl. Acad. Sci. USA 107:20021 (2010).
8. W. Debinski, and D. M. Gibo, Molecular expression analysis of restrictive receptor for interleukin 13, a brain tumor-associated cancer/testis antigen. Mol. Med. 6:440 (2000).
9. H. Liu et al., Interleukin-13 sensitivity and receptor phenotypes of human glial cell lines: non-neoplastic glia and low-grade astrocytoma differ from malignant glioma. Cancer Immunol Immunother. CII 49:319 (2000).
10. C. P. Theuer, et al., A recombinant form of Pseudomonas exotoxin directed at the epidermal growth factor receptor that is cytotoxic without requiring proteolytic processing. J. Biol. Chem. 267:16872 (1992).
11. I. A. Lorimer et al., Recombinant immunotoxins specific for a mutant epidermal growth factor receptor: targeting with a single chain antibody variable domain isolated by phage display. Proc. Natl. Acad. Sci. USA 93:14815 (1996).
12. T. F. Liu et al., Diphtheria toxin-epidermal growth factor fusion protein and Pseudomonas exotoxin-interleukin 13 fusion protein exert synergistic toxicity against human glioblastoma multiforme cells. Bioconjugate Chem. 14:1107 (2003).
13. F. Ciardiello, and G. Tortora, EGFR antagonists in cancer treatment. NEJM 358:1160 (2008).
14. R. J. Kreitman, and I. Pastan, Immunotoxins in the treatment of hematologic malignancies. Curr. Drug Targets 7:1301 (2006).
15. R. J. Kreitman et al., Responses in refractory hairy cell leukemia to a recombinant immunotoxin. Blood 94:3340 (1999).
16. R. J. Kreitman et al., Phase I trial of recombinant immunotoxin anti-Tac(Fv)-PE38 (LMB-2) in patients with hematologic malignancies. J. Clin. Oncol. 18:1622 (2000).
17. R. J. Kreitman et al., Phase I trial of recombinant immunotoxin RFB4(dsFv)-PE38 (BL22) in patients with B-cell malignancies. J. Clin. Oncol. 23:6719 (2005).
18. R. J. Kreitman et al., Phase II trial of recombinant immunotoxin RFB4(dsFv)-PE38 (BL22) in patients with hairy cell leukemia. J. Clin. Oncol. 27:2983 (2009).
19. W. Debinski, et al., A novel chimeric protein composed of interleukin 13 and Pseudomonas exotoxin is highly cytotoxic to human carcinoma cells expressing receptors for interleukin 13 and interleukin 4. J. Biol. Chem. 270:16775 (1995).
20. W. Debinski, et al., Human glioma cells overexpress receptors for interleukin 13 and are extremely sensitive to a novel chimeric protein composed of interleukin 13 and pseudomonas exotoxin. Clin. Cancer Res. 1:1253 (1995).
21. S. R. Husain, et al., Interleukin-13 receptor as a unique target for anti-glioblastoma therapy. Int. J Cancer 92:168 (2001).
22. S. Kunwar et al., Phase III randomized trial of CED of IL13-PE38QQR vs Gliadel wafers for recurrent glioblastoma. Neuro-Oncol. 12:871 (2010).
23. Y. Wang, and T. Jiang, Understanding high grade glioma: molecular mechanism, therapy and comprehensive management. Cancer Lett. 331:139 (2013).
24. P. H. Huang, et al., Oncogenic EGFR signaling networks in glioma. Sci STKE 2: re6 (2009).
25. P. Holliger, and P. J. Hudson, Engineered antibody fragments and the rise of single domains. Nature Biotechnol. 23:1126 (2005).
26. J. A. van de Water et al., Therapeutic stem cells expressing variants of EGFR-specific nanobodies have antitumor effects. Proc. Natl. Acad. Sci. USA 109:16642 (2012).
27. R. C. Roovers et al., Efficient inhibition of EGFR signaling and of tumour growth by antagonistic anti-EFGR Nanobodies. Cancer Immunol Immunother. CII 56:303 (2007).
28. T. M. Kauer, et al., Encapsulated therapeutic stem cells implanted in the tumor resection cavity induce cell death in gliomas. Nature Neurosci. 15:197 (2012).
29. K. Shah et al., Bimodal viral vectors and in vivo imaging reveal the fate of human neural stem cells in experimental glioma model. J Neurosci. 28:4406 (2008).
30. N. Kock, R. et al., Tumor therapy mediated by lentiviral expression of shBcl-2 and S-TRAIL. Neoplasia 9:435 (2007).
31. L. S. Sasportas et al., Assessment of therapeutic efficacy and fate of engineered human mesenchymal stem cells for cancer therapy. Proc. Natl. Acad. Sci. USA 106:4822 (2009).
32. H. Wakimoto et al., Human glioblastoma-derived cancer stem cells: establishment of invasive glioma models and treatment with oncolytic herpes simplex virus vectors. Cancer Res. 69:3472 (2009).
33. T. Bagci-Onder, et al., A dual PI3K/mTOR inhibitor, PI-103, cooperates with stem cell-delivered TRAIL in experimental glioma models. Cancer Res. 71:154 (2011).
34. S. Shulga-Morskoy, B. E. Rich, Bioactive IL7-diphtheria fusion toxin secreted by mammalian cells. Protein engineering, design & selection. PEDS 18:25 (2005).
35. R. Haq et al., BCL2A1 is a lineage-specific antiapoptotic melanoma oncogene that confers resistance to BRAF inhibition. Proc. Natl. Acad. Sci. USA 110:4321 (2013).
36. A. F. Gazdar et al., Characterization of paired tumor and non-tumor cell lines established from patients with breast cancer. Int. J. Cancer 78:766 (1998).
37. E. Y. Lasfargues, et al., Isolation of two human tumor epithelial cell lines from solid breast carcinomas. J. Natl. Cancer Inst. 61:967 (1978).
38. A. Villa, et al., Establishment and properties of a growth factor-dependent, perpetual neural stem cell line from the human CNS. Exp. Neurol. 161:67 (2000).
39. K. Kohno, T. Uchida, Highly frequent single amino acid substitution in mammalian elongation factor 2 (EF-2) results in expression of resistance to EF-2-ADP-ribosylating toxins. J. Biol. Chem. 262:12298 (1987).
40. A. H. Wu, and W. C. Low, Molecular cloning and identification of the human interleukin 13 alpha 2 receptor (IL-13Ra2) promoter. Neuro-Oncol. 5:179 (2003).
41. D. M. Gill, et al., Studies on the mode of action of diphtheria toxin. VII. Toxin-stimulated hydrolysis of nicotinamide adenine dinucleotide in mammalian cell extracts. J. Exp. Med. 129:1 (1969).
42. B. H. Iglewski, and D. Kabat, NAD-dependent inhibition of protein synthesis by Pseudomonas aeruginosa toxin. Proc. Natl. Acad. Sci. USA 72:2284 (1975).
43. M. Yamaizumi, et al., One molecule of diphtheria toxin fragment A introduced into a cell can kill the cell. Cell 15:245 (1978).
44. M. F. Corsten, and K. Shah, Therapeutic stem-cells for cancer treatment: hopes and hurdles in tactical warfare. Lancet Oncology 9:376 (2008).
45. A. U. Ahmed, et al., The use of neural stem cells in cancer gene therapy: predicting the path to the clinic. Curr. Opin. Mol. Ther. 12:546 (2010).
46. K. S. Aboody, et al., Stem and progenitor cell-mediated tumor selective gene therapy. Gene therapy 15:739 (2008).
47. B. T. Foley, et al., Mutations in the elongation factor 2 gene which confer resistance to diphtheria toxin and Pseudomonas exotoxin A. Genetic and biochemical analyses. J. Biol. Chem. 270:23218 (1995).
48. A. R. Asthagiri, et al., Advances in brain tumor surgery. Neurol. cfin. 25:975 (2007).
49. M. Lacroix et al., A multivariate analysis of 416 patients with glioblastoma multiforme: prognosis, extent of resection, and survival. J. Neurosurg. 95:190 (2001).
50. C. Altaner et al., Complete regression of glioblastoma by mesenchymal stem cells mediated prodrug gene therapy simulating clinical therapeutic scenario. Int. J Cancer 134:1458 (2014).
51. L. Bello et al., Suppression of malignant glioma recurrence in a newly developed animal model by endogenous inhibitors. Clin. Cancer Res. 8:3539 (2002).
52. R. S. Herbst, Review of epidermal growth factor receptor biology. Intl. J. Radiat. Oncol. Biol. Phys. 59:21 (2004).
53. V. L. Jacobs, et al., Current review of in vivo GBM rodent models: emphasis on the CNS-1 tumour model. ASN Neuro 3:e00063 (2011).
54. T. Fujisawa, et al., Histone modification enhances the effectiveness of IL-13 receptor targeted immunotoxin in murine models of human pancreatic cancer. J. Translational Med. 9:37 (2011).
55. Z. Wang et al., Development of a Non-integrating Rev-dependent Lentiviral Vector Carrying Diphtheria Toxin A Chain and Human TRAF6 to Target HIV Reservoirs. Gene Ther. 17: 1063-1076 (2010)

```
Mutant EF2 oligo sequence (SEQ. ID. No: 1):
tgtggggatctggccccctctgcggtggatggcgtcggcgtgca
PE fragment 1 nucleic acid coding sequence
(SEQ. ID. No: 2):
atcgacaacgccctcagcatcaccagcgacggcctgaccatccgcctc
gaaggtggcgtcgagccgaacaagccggtgcgctacagctacacgcgc
caggcgcgcggcagttggtcgctgaactggctggtgccgatcggccac
gagaagccttcgaacatcaaggtgttcatccacgaactgaacgccggt
aaccagctcagccacatgtcgccgatctacaccatcgagatgggcgac
gagttgctggcgaagctggcgcgcgatgccaccttcttcgtcagggcg
cacgagagcaacgagatgcagccgacgctcgccatcagccatgccggg
gtcagcgtggtcatggcccaggccc
Legend for Fusion protein nucleotide sequences:
Bold, lower case- Signal Sequence for the
secretion
Underline, lower case- Linker sequences
Upper case- coding sequences of EGFR-Nb or IL-13
Italics, lower case- coding sequences of PE
ENb-PE comprising coding sequences of EGFR-Nb
(SEQ. ID. No: 3):
atgacagtgctggcgccagcctggagcccaacaacctatctcctcctg
ctgctgctgctgagctcgggactcagtgggTCAGATCCGCTAGCATGA
CAGTGCTGGCGCCAGCCTGGAGCCCAACAACCTATNTCCTCCTGCTGC
TGCTGCTGAGCTCGGGACTCAGTGGGGAATTCCAGGTAAAGCTGGAGG
AGTCTGGGGGAGGGTCGGTGCAGACTGGGGGCTCTCTGAGACTCACCT
GTGCAGCCTCTGGACGCACTTCAAGGAGCTATGGCATGGGATGGTTCC
GCCAGGCTCCAGGGAAGGAGCGTGAGTTTGTATCAGGTATTAGTTGGA
GGGGTGATAGTACAGGCTATGCAGACTCCGTGAAGGGCCGATTCACCA
TCTCCAGAGACAACGCCAAGAACACGGTGGATCTGCAAATGAACAGCC
TGAAACCTGAGGACACGGCCATTTATTATTGTGCGGCGGCCGCAGGTA
GTGCCTGGTACGGAACATTGTATGAATACGACTACTGGGGCCAGGGGA
CCCAGGTCACCGTCTCCTCAGGCGGTGGAGGCAGCGGTGGCGGGGGAT
CCGAGGTGCAGCTGGTGGAGTCTGGGGGAGGATTGGTGCAGGCTGGGG
GCTCTCTGAGACTCTCCTGTGCAGCCTCTGGACGCACCTTCAGTAGCT
ATGCCATGGGCTGGTTCCGCCAGGCTCCAGGGAAGGAGCGTGAGTTTG
TAGTAGCTATTAACTGGAGTAGTGGTAGCACATACTATGCAGACTCCG
TGAAGGGCCGATTCACCATCTCCAGAGACAACGCCAAGAACACGATGT
ATCTGCAGATGAACAGCCTGAAACCTGAGGACACGGCCGTTTATTACT
GTGCAGCAGGGTATCAGATTAATAGTGGTAATTACAACTTTAAAGACT
ATGAGTATGACTACTGGGGCCAGGGGACCCAGGTCACCGTCTCCTCAg
atatcggatctaccggtggatctggaaaacccggatctggagagggat
ctaccatcgacaacgccctcagcatcaccagcgacggcctgaccatcc
gcctcgaaggtggcgtcgagccgaacaagccggtgcgctacagctaca
cgcgccaggcgcgcggcagttggtcgctgaactggctggtgccgatcg
gccacgagaagccttcgaacatcaaggtgttcatccacgaactgaacg
ccggtaaccagctcagccacatgtcgccgatctacaccatcgagatgg
gcgacgagttgctggcgaagctggcgcgcgatgccaccttcttcgtca
gggcgcacgagagcaacgagatgcagccgacgctcgccatcagccatg
ccggggtcagcgtggtcatggcccaggccc
IL13-PE comprising coding sequence of IL13
(SEQ. ID. No: 4):
atgacagtgctggcgccagcctggagcccaacaacctatctcctcctg
ctgctgctgctgagctcgggactcagtgggATGGCGCTTTTGTTGACC
ACGGTCATTGCTCTCACTTGCCTTGGCGGCTTTGCCTCCCCAGGCCCT
GTGCCTCCCTCTACAGCCCTCAGGGAGCTCATTGAGGAGCTGGTCAAC
ATCACCCAGAACCAGAAGGCTCCGCTCTGCAATGGCAGCATGGTATGG
AGCATCAACCTGACAGCTGGCATGTACTGTGCAGCCCTGGAATCCCTG
ATCAACGTGTCAGGCTGCAGTGCCATCGAGAAGACCCAGAGGATGCTG
AGCGGATTCTGCCCGCACAAGGTCTCAGCTGGGCAGTTTTCCAGCTTG
CATGTCCGAGACACCAAAATCGAGGTGGCCCAGTTTGTAAAGGACCTG
CTCTTACATTTAAAGAAACTTTTTCGCGAGGGACGGTTCAACTGAAAC
TTCGAAAGCATCATTATTTGCAGAGACAGGACCTGACgatatcggatc
taccggtggatctggaaaacccggatctggagagggatctaccatcga
caacgccctcagcatcaccagcgacggcctgaccatccgcctcgaagg
tggcgtcgagccgaacaagccggtgcgctacagctacacgcgccaggc
gcgcggcagttggtcgctgaactggctggtgccgatcggccacgagaa
gccttcgaacatcaaggtgttcatccacgaactgaacgccggtaacca
gctcagccacatgtcgccgatctacaccatcgagatgggcgacgagtt
gctggcgaagctggcgcgcgatgccaccttcttcgtcagggcgcacga
gagcaacgagatgcagccgacgctcgccatcagccatgccggggtcag
cgtggtcatggcccaggccc
Human EF-2 protein sequence
(SEQ. ID. No: 5)
MVNFTVDQIRAIMDKKANIRNMSVIAHVDHGKSTLTDSLVCKAGIIAS
ARAGETRFTDTRKDEQERCITIKSTAISLFYELSENDLNFIKQSKDGA
GFLINLIDSPGHVDFSSEVTAALRVTDGALVVVDCVSGVCVQTETVLR
QAIAERIKPVLMMNKMDRALLELQLEPEELYQTFQRIVENVNVIISTY
GEGESGPMGNIMIDPVLGTVGFGSGLHGWAFTLKQFAEMYVAKFAAKG
EGQLGPAERAKKVEDMMKKLWGDRYFDPANGKFSKSATSPEGKKLPRT
FCQLILDPIFKVFDAIMNFKKEETAKLIEKLDIKLDSEDKDKEGKPLL
KAVMRRWLPAGDALLQMITIHLPSPVTAQKYRCELLYEGPPDDEAAMG
IKSCDPKGPLMMYISKMVPTSDKGRFYAFGRVFSGLVSTGLKVRIMGP
NYTPGKKEDLYLKPIQRTILMMGRYVEPIEDVPCGNIVGLVGVDQFLV
KTGTITTFEHAHNMRVMKFSVSPVVRVAVEAKNPADLPKLVEGLKRLA
KSDPMVQCIIEESGEHIIAGAGELHLEICLKDLEEDHACIPIKKSDPV
VSYRETVSEESNVLCLSKSPNKHNRLYMKARPFPDGLAEDIDKGEVSA
RQELKQRARYLAEKYEWDVAEARKIWCFGPDGTGPNILTDITKGVQYL
NEIKDSVVAGFQWATKEGALCEENMRGVRFDVHDVTLHADAIHRGGGQ
IIPTARRCLYASVLTAQPRLMEPIYLVEIQCPEQVVGGIYGVLNRKRG
HVFEESQVAGTPMFVVKAYLPVNESFGFTADLRSNTGGQAFPQCVFDH
WQILPGDPFDNSSRPSQVVAETRKRKGLKEGIPALDNFLDKL
Coding Sequence for wild type human EF-2 protein
(SEQ. ID. No: 6)
      atggtga acttcacggt agaccagatc cgcgccatca
tggacaagaa ggccaacatc cgcaacatgt ctgtcatcgc
ccacgtggac catggcaagt ccacgctgac agactccctg
gtgtgcaagg cgggcatcat cgcctcggcc cgggccgggg
agacacgctt cactgatacc cggaaggacg agcaggagcg
ttgcatcacc atcaagtcaa ctgccatctc cctcttctac
gagctctcgg agaatgactt gaacttcatc aagcagagca
aggacggtgc cggcttcctc atcaacctca ttgactcccc
cgggcatgtc gacttctcct cggaggtgac tgctgccctc
cgagtcaccg atggcgcatt ggtggtggtg gactgcgtgt
caggcgtgtg cgtgcagacg gagacagtgc tgcggcaggc
cattgccgag cgcatcaagc ctgtgctgat gatgaacaag
atggaccgcg ccctgctgga gctgcagctg gagcccgagg
agctctacca gactttccag cgcatcgtgg agaacgtgaa
cgtcatcatc tccacctacg gcgagggcga gagcggcccc
atgggcaaca tcatgatcga tcctgtcctc ggtaccgtgg
gctttgggtc tggcctccac gggtgggcct tcaccctgaa
gcagtttgcc gagatgtatg tggccaagtt cgccgccaag
ggggagggcc agttggggcc tgccgagcgg gccaagaaag
tagaggacat gatgaagaag ctgtggggtg acaggtactt
tgacccagcc aacggcaagt tcagcaagtc agccaccagc
cccgaaggga agaagctgcc acgcaccttc tgccagctga
tcctggaccc catcttcaag gtgtttgatg cgatcatgaa
tttcaagaaa gaggagacag caaaactgat agagaaactg
gacatcaaac tggacagcga ggacaaggac aaagaaggca
aacccctgct gaaggctgtg atgcgccgct ggctgcctgc
cggagacgcc ttgttgcaga tgatcaccat ccacctgccc
tcccctgtga cggcccagaa gtaccgctgc gagctcctgt
acgaggggcc cccggacgac gaggctgcca tgggcattaa
aagctgtgac cccaaaggcc ctcttatgat gtatatttcc
aaaatggtgc caacctccga caaaggtcgg ttctacgcct
ttggacgagt cttctcgggg ctggtctcca ctggcctgaa
ggtcaggatc atggggccca actatacccc tgggaagaag
gaggacctct acctgaagcc aatccagaga acaatcttga
tgatgggccg ctacgtggag cccatcgagg atgtgccttg
tgggaacatt gtgggcctcg tgggcgtgga ccagttcctg
gtgaagacgg gcaccatcac caccttcgag cacgcgcaca
acatgcgggt gatgaagttc agcgtcagcc ctgttgtcag
agtggccgtg gaggccaaga acccggctga cctgcccaag
ctggtggagg ggctgaagcg gctggccaag tccgacccca
tggtgcagtg catcatcgag gagtcgggag agcatatcat
cgcgggcgcc ggcgagctgc acctggagat ctgcctgaag
gacctggagg aggaccacgc ctgcatcccc atcaagaaat
ctgacccggt cgtctcgtac cgcgagacgg tcagtgaaga
gtcgaacgtg ctctgcctct ccaagtcccc caacaagcac
aaccggctgt acatgaaggc gcggcccttc cccgacggcc
tggccgagga catcgataaa ggcgaggtgt ccgcccgtca
ggagctcaag cagcgggcgc gctacctggc cgagaagtac
gagtgggacg tggctgaggc ccgcaagatc tggtgctttg
ggcccgacgg caccggcccc aacatcctca ccgacatcac
caagggtgtg cagtacctca acgagatcaa ggacagtgtg
gtggccggct tccagtgggc caccaaggag ggcgcactgt
gtgaggagaa catgcggggt gtgcgcttcg acgtccacga
cgtcaccctg cacgccgacg ccatccaccg cggagggggc
cagatcatcc ccacagcacg gcgctgcctc tatgccagtg
tgctgaccgc ccagccacgc ctcatggagc ccatctacct
tgtggagatc cagtgtccag agcaggtggt cggtggcatc
tacggggttt tgaacaggaa gcggggccac gtgttcgagg
agtcccaggt ggccggcacc cccatgtttg tggtcaaggc
ctatctgccc gtcaacgagt cctttggctt caccgctgac
ctgaggtcca acacgggcgg ccaggcgttc ccccagtgtg
tgtttgacca ctggcagatc ctgcccggag accccttcga
caacagcagc cgccccagcc aggtggtggc ggagacccgc
aagcgcaagg gcctgaaaga aggcatccct gccctggaca
acttcctgga caaattgtag
Coding Sequence with mutated G→A (at condon717)
sequence for human EF-2 protein
(SEQ. ID. No: 7)
      atggtga acttcacggt agaccagatc cgcgccatca
tggacaagaa ggccaacatc cgcaacatgt ctgtcatcgc
ccacgtggac catggcaagt ccacgctgac agactccctg
gtgtgcaagg cgggcatcat cgcctcggcc cgggccgggg
agacacgctt cactgatacc cggaaggacg agcaggagcg
ttgcatcacc atcaagtcaa ctgccatctc cctcttctac
gagctctcgg agaatgactt gaacttcatc aagcagagca
aggacggtgc cggcttcctc atcaacctca ttgactcccc
cgggcatgtc gacttctcct cggaggtgac tgctgccctc
cgagtcaccg atggcgcatt ggtggtggtg gactgcgtgt
caggcgtgtg cgtgcagacg gagacagtgc tgcggcaggc
cattgccgag cgcatcaagc ctgtgctgat gatgaacaag
atggaccgcg ccctgctgga gctgcagctg gagcccgagg
agctctacca gactttccag cgcatcgtgg agaacgtgaa
cgtcatcatc tccacctacg gcgagggcga gagcggcccc
atgggcaaca tcatgatcga tcctgtcctc ggtaccgtgg
gctttgggtc tggcctccac gggtgggcct tcaccctgaa
gcagtttgcc gagatgtatg tggccaagtt cgccgccaaa
ggggagggcc agttggggcc tgccgagcgg gccaagaaag
tagaggacat gatgaagaag ctgtggggtg acaggtactt
tgacccagcc aacggcaagt tcagcaagtc agccaccagc
cccgaaggga agaagctgcc acgcaccttc tgccagctga
tcctggaccc catcttcaag gtgtttgatg cgatcatgaa
tttcaagaaa gaggagacag caaaactgat agagaaactg
gacatcaaac tggacagcga ggacaaggac aaagaaggca
aacccctgct gaaggctgtg atgcgccgct ggctgcctgc
cggagacgcc ttgttgcaga tgatcaccat ccacctgccc
tcccctgtga cggcccagaa gtaccgctgc gagctcctgt
acgaggggcc cccggacgac gaggctgcca tgggcattaa
aagctgtgac cccaaaggcc ctcttatgat gtatatttcc
aaaatggtgc caacctccga caaaggtcgg ttctacgcct
ttggacgagt cttctcgggg ctggtctcca ctggcctgaa
ggtcaggatc atggggccca actatacccc tgggaagaag
gaggacctct acctgaagcc aatccagaga acaatcttga
tgatgggccg ctacgtggag cccatcgagg atgtgccttg
tgggaacatt gtgggcctcg tgggcgtgga ccagttcctg
gtgaagacgg gcaccatcac caccttcgag cacgcgcaca
acatgcgggt gatgaagttc agcgtcagcc ctgttgtcag
agtggccgtg gaggccaaga acccggctga cctgcccaag
ctggtggagg ggctgaagcg gctggccaag tccgacccca
tggtgcagtg catcatcgag gagtcgggag agcatatcat
cgcgggcgcc ggcgagctgc acctggagat ctgcctgaag
gacctggagg aggaccacgc ctgcatcccc atcaagaaat
ctgacccggt cgtctcgtac cgcgagacgg tcagtgaaga
gtcgaacgtg ctctgcctct ccaagtcccc caacaagcac
aaccggctgt acatgaaggc gcggcccttc cccgacggcc
tggccgagga catcgataaa ggcgaggtgt ccgcccgtca
ggagctcaag cagcgggcgc gctacctggc cgagaagtac
gagtgggacg tggctgaggc ccgcaagatc tggtgctttg
ggcccgacgg caccggcccc aacatcctca ccgacatcac
caagggtgtg cagtacctca acgagatcaa ggacagtgtg
gtggccggct tccagtgggc caccaaggag ggcgcactgt
gtgaggagaa catgcggggt gtgcgcttcg acgtccacga
cgtcaccctg cacgccgacg ccatccaccg cggagggggc
cagatcatcc ccacagcacg gcgctgcctc tatgccagtg
tgctgaccgc ccagccacgc ctcatggagc ccatctacct
tgtggagatc cagtgtccag agcaggtggt cggtggcatc
tacggggttt tgaacaggaa gcggggccac gtgttcgagg
agtcccaggt ggccggcacc cccatgtttg tggtcaaggc
ctatctgccc gtcaacgagt cctttggctt caccgctgac
ctgaggtcca acacgggcgg ccaggcgttc ccccagtgtg
tgtttgacca ctggcagatc ctgcccggag accccttcga
caacagcagc cgccccagcc aggtggtggc ggagacccgc
aagcgcaagg gcctgaaaga aggcatccct gccctggaca
acttcctgga caaattgtag
Signal peptide
(SEQ. ID. No: 8)
atgacagtgctggcgccagcctggagcccaacaacctatctcctcctg
ctgctgctgctgagctcgggactcagtggg
ENb
(SEQ. ID. No: 17)
TCAGATCCGCTAGCATGACAGTGCTGGCGCCAGCCTGGAGCCCAACAA
CCTATNTCCTCCTGCTGCTGCTGCTGAGCTCGGGACTCAGTGGGGAAT
TCCAGGTAAAGCTGGAGGAGTCTGGGGGAGGGTCGGTGCAGACTGGGG
GCTCTCTGAGACTCACCTGTGCAGCCTCTGGACGCACTTCAAGGAGCT
ATGGCATGGGATGGTTCCGCCAGGCTCCAGGGAAGGAGCGTGAGTTTG
TATCAGGTATTAGTTGGAGGGGTGATAGTACAGGCTATGCAGACTCCG
TGAAGGGCCGATTCACCATCTCCAGAGACAACGCCAAGAACACGGTGG
ATCTGCAAATGAACAGCCTGAAACCTGAGGACACGGCCATTTATTATT
GTGCGGCGGCCGCAGGTAGTGCCTGGTACGGAACATTGTATGAATACG
ACTACTGGGGCCAGGGGACCCAGGTCACCGTCTCCTCAGGCGGTGGAG
GCAGCGGTGGCGGGGGATCCGAGGTGCAGCTGGTGGAGTCTGGGGGAG
GATTGGTGCAGGCTGGGGGCTCTCTGAGACTCTCCTGTGCAGCCTCTG
GACGCACCTTCAGTAGCTATGCCATGGGCTGGTTCCGCCAGGCTCCAG
GGAAGGAGCGTGAGTTTGTAGTAGCTATTAACTGGAGTAGTGGTAGCA
CATACTATGCAGACTCCGTGAAGGGCCGATTCACCATCTCCAGAGACA
ACGCCAAGAACACGATGTATCTGCAGATGAACAGCCTGAAACCTGAGG
ACACGGCCGTTTATTACTGTGCAGCAGGGTATCAGATTAATAGTGGTA
ATTACAACTTTAAAGACTATGAGTATGACTACTGGGGCCAGGGGACCC
AGGTCACCGTCTCCTCA
IL-13
(SEQ. ID. No: 18)
ATGGCGCTTTTGTTGACCACGGTCATTGCTCTCACTTGCCTTGGCGGC
TTTGCCTCCCCAGGCCCTGTGCCTCCCTCTACAGCCCTCAGGGAGCTC
ATTGAGGAGCTGGTCAACATCACCCAGAACCAGAAGGCTCCGCTCTGC
AATGGCAGCATGGTATGGAGCATCAACCTGACAGCTGGCATGTACTGT
GCAGCCCTGGAATCCCTGATCAACGTGTCAGGCTGCAGTGCCATCGAG
AAGACCCAGAGGATGCTGAGCGGATTCTGCCCGCACAAGGTCTCAGCT
GGGCAGTTTTCCAGCTTGCATGTCCGAGACACCAAAATCGAGGTGGCC
CAGTTTGTAAAGGACCTGCTCTTACATTTAAAGAAACTTTTTCGCGAG
GGACGGTTCAACTGAAACTTCGAAAGCATCATTATTTGCAGAGACAGG
ACCTGAC
PE fragment 2 nucleic acid coding sequence
(SEQ. ID. No: 19):
GGCGGCAGCCTGGCCGCGCTGACCGCGCACCAGGCTTGCCACCTGCCG
CTGGAGACTTTCACCCGTCATCGCCAGCCGCGCGGCTGGGAACAACTG
GAGCAGTGCGGCTATCCGGTGCAGCGGCTGGTCGCCCTCTACCTGGCG
GCGCGGCTGTCGTGGAACCAGGTCGACCAGGTGATCCGCAACGCCCTG
GCCAGCCCCGGCAGCGGCGGCGACCTGGGCGAAGCGATCCGCGAGCAG
CCGGAGCAGGCCCGTCTGGCCCTGACCCTGGCCGCCGCCGAGAGCGAG
CGCTTCGTCCGGCAGGGCACCGGCAACGACGAGGCCGGCGCGGCCAAC
GCCGACGTGGTGAGCCTGACCTGCCCGGTCGCCGCCGGTGAATGCGCG
GGCCCGGCGGACAGCGGCGACGCCCTGCTGGAGCGCAACTATCCCACT
GGCGCGGAGTTCCTCGGCGACGGCGGCGACGTCAGCTTCAGCACCCGC
GGCACGCAGAACTGGACGGTGGAGCGGCTGCTCCAGGCGCACCGCCAA
CTGGAGGAGCGCGGCTATGTGTTCGTCGGCTACCACGGCACCTTCCTC
GAAGCGGCGCAAAGCATCGTCTTCGGCGGGGTGCGCGCGCGCAGCCAG
GACCTCGACGCGATCTGGCGCGGTTTCTATATCGCCGGCGATCCGGCG
CTGGCCTACGGCTACGCCCAGGACCAGGAACCCGACGCACGCGGCCGG
ATCCGCAACGGTGCCCTGCTGCGGGTCTATGTGCCGCGCTCGAGCCTG
CCGGGCTTCTACCGCACCAGCCTGACCCTGGCCGCGCCGGAGGCGGCG
GGCGAGGTCGAACGGCTGATCGGCCATCCGCTGCCGCTGCGCCTGGAC
GCCATCACCGGCCCCGAGGAGGAAGGCGGGCGCCTGGAGACCATTCTC
GGCTGGCCGCTGGCCGAGCGCACCGTGGTGATTCCCTCGGCGATCCCC
ACCGACCCGCGCAACGTCGGCGGCGACCTCGACCCGTCCAGCATCCCC
GACAAGGAACAGGCGATCAGCGCCCTGCCGGACTACGCCAGCCAGCCC
GGCAAACCGCCGCGCGAGGACCTGAAGTAA
PE fragment 2, AMINO ACID SEQUENCE
(SEQ. ID. No: 20):
GGSLAALTAHQACHLPLETFTRHRQPRGWEQLEQCGYPVQRLVALYLA
ARLSWNQVDQVIRNALASPGSGGDLGEAIREQPEQARLALTLAAAESE
RFVRQGTGNDEAGAANADVVSLTCPVAAGECAGPADSGDALLERNYPT
GAEFLGDGGDVSFSTRGTQNWTVERLLQAHRQLEERGYVFVGYHGTFL
EAAQSIVEGGVRARSQDLDAIWRGFYIAGDPALAYGYAQDQEPDARGR
IRNGALLRVYVPRSSLPGFYRTSLTLAAPEAAGEVERLIGHPLPLRLD
AITGPEEEGGRLETILGWPLAERTVVIPSAIPTDPRNVGGDLDPSSIP
DKEQAISALPDYASQPGKPPREDLK
gi|15596345|ref|NP_249839.1| PE full-length
protein
(SEQ. ID. NO: 21)
MHLTPHWIPLVASLGLLAGGSFASAAEEAFDLWNECAKACVLDLKDGV
RSSRMSVDPAIADTNGQGVLHYSMVLEGGNDALKLAIDNALSITSDGL
TIRLEGGVEPNKPVRYSYTRQARGSWSLNWLVPIGHEKPSNIKVFIHE
LNAGNQLSHMSPIYTIEMGDELLAKLARDATFFVRAHESNEMQPTLAI
SHAGVSVVMAQAQPRREKRWSEWASGKVLCLLDPLDGVYNYLAQQRCN
LDDTWEGKIYRVLAGNPAKHDLDIKPTVISHRLHFPEGGSLAALTAHQ
ACHLPLETFTRHRQPRGWEQLEQCGYPVQRLVALYLAARLSWNQVDQV
IRNALASPGSGGDLGEAIREQPEQARLALTLAAAESERFVRQGTGNDE
AGAASADVVSLTCPVAAGECAGPADSGDALLERNYPTGAEFLGDGGDI
SFSTRGTQNWTVERLLQAHRQLEERGYVFVGYHGTFLEAAQSIVEGGV
RARSQDLDAIWRGFYIAGDPALAYGYAQDQEPDARGRIRNGALLRVYV
PRSSLPGFYRTGLTLAAPEAAGEVERLIGHPLPLRLDAITGPEEEGGR
LETILGWPLAERTVVIPSAIPTDPRNVGGDLDPSSIPDKEQAISALPD
YASQPGKPPREDLK
European Nucleotide Archive |AAB59097|AAB59097.1
Pseudomonas aeruginosa exotoxin type A
(SEQ. ID. NO: 22)
ATGCACCTGATACCCCATTGGATCCCCCTGGTCGCCAGCCTCGGCCTG
CTCGCCGGCGGCTCGTCCGCGTCCGCCGCCGAGGAAGCCTTCGACCTC
TGGAACGAATGCGCCAAAGCCTGCGTGCTCGACCTCAAGGACGGCGTG
CGTTCCAGCCGCATGAGCGTCGACCCGGCCATCGCCGACACCAACGGC
CAGGGCGTGCTGCACTACTCCATGGTCCTGGAGGGCGGCAACGACGCG
CTCAAGCTGGCCATCGACAACGCCCTCAGCATCACCAGCGACGGCCTG
ACCATCCGCCTCGAAGGCGGCGTCGAGCCGAACAAGCCGGTGCGCTAC
AGCTACACGCGCCAGGCGCGCGGCAGTTGGTCGCTGAACTGGCTGGTA
CCGATCGGCCACGAGAAGCCCTCGAACATCAAGGTGTTCATCCACGAA
CTGAACGCCGGCAACCAGCTCAGCCACATGTCGCCGATCTACACCATC
GAGATGGGCGACGAGTTGCTGGCGAAGCTGGCGCGCGATGCCACCTTC
TTCGTCAGGGCGCACGAGAGCAACGAGATGCAGCCGACGCTCGCCATC
AGCCATGCCGGGGTCAGCGTGGTCATGGCCCAGACCCAGCCGCGCCGG
GAAAAGCGCTGGAGCGAATGGGCCAGCGGCAAGGTGTTGTGCCTGCTC
GACCCGCTGGACGGGGTCTACAACTACCTCGCCCAGCAACGCTGCAAC
CTCGACGATACCTGGGAAGGCAAGATCTACCGGGTGCTCGCCGGCAAC
CCGGCGAAGCATGACCTGGACATCAAACCCACGGTCATCAGTCATCGC
CTGCACTTTCCCGAGGGCGGCAGCCTGGCCGCGCTGACCGCGCACCAG
GCTTGCCACCTGCCGCTGGAGACTTTCACCCGTCATCGCCAGCCGCGC
GGCTGGGAACAACTGGAGCAGTGCGGCTATCCGGTGCAGCGGCTGGTC
GCCCTCTACCTGGCGGCGCGGCTGTCGTGGAACCAGGTCGACCAGGTG
ATCCGCAACGCCCTGGCCAGCCCCGGCAGCGGCGGCGACCTGGGCGAA
GCGATCCGCGAGCAGCCGGAGCAGGCCCGTCTGGCCCTGACCCTGGCC
GCCGCCGAGAGCGAGCGCTTCGTCCGGCAGGGCACCGGCAACGACGAG
GCCGGCGCGGCCAACGCCGACGTGGTGAGCCTGACCTGCCCGGTCGCC
GCCGGTGAATGCGCGGGCCCGGCGGACAGCGGCGACGCCCTGCTGGAG
CGCAACTATCCCACTGGCGCGGAGTTCCTCGGCGACGGCGGCGACGTC
AGCTTCAGCACCCGCGGCACGCAGAACTGGACGGTGGAGCGGCTGCTC
CAGGCGCACCGCCAACTGGAGGAGCGCGGCTATGTGTTCGTCGGCTAC
CACGGCACCTTCCTCGAAGCGGCGCAAAGCATCGTCTTCGGCGGGGTG
CGCGCGCGCAGCCAGGACCTCGACGCGATCTGGCGCGGTTTCTATATC
GCCGGCGATCCGGCGCTGGCCTACGGCTACGCCCAGGACCAGGAACCC
GACGCACGCGGCCGGATCCGCAACGGTGCCCTGCTGCGGGTCTATGTG
CCGCGCTCGAGCCTGCCGGGCTTCTACCGCACCAGCCTGACCCTGGCC
GCGCCGGAGGCGGCGGGCGAGGTCGAACGGCTGATCGGCCATCCGCTG
CCGCTGCGCCTGGACGCCATCACCGGCCCCGAGGAGGAAGGCGGGCGC
CTGGAGACCATTCTCGGCTGGCCGCTGGCCGAGCGCACCGTGGTGATT
CCCTCGGCGATCCCCACCGACCCGCGCAACGTCGGCGGCGACCTCGAC
CCGTCCAGCATCCCCGACAAGGAACAGGCGATCAGCGCCCTGCCGGAC
TACGCCAGCCAGCCCGGCAAACCGCCGCGCGAGGACCTGAAGTAA
PE fragment 2, AMINO ACID SEQUENCE
(SEQ. ID. No: 23):
IDNALSITSDGLTIRLEGGVEPNKPVRYSYTRQARGSWSLNWLVPIGH
EKPSNIKVFIHELNAGNQLSHMSPIYTIEMGDELLAKLARDATFFVRA
HESNEMQPTLAISHAGVSVVMAQA
```

1. An engineered mammalian cell that is resistant to a Pseudonomas exotoxin (PE), and also expresses and secretes PE.

2.-4. (canceled)

5. The engineered mammalian cell of claim 1, wherein the engineered mammalian cell comprises a modified nucleic acid sequence encoding an elongation factor 2 protein (EF-2).

6. The engineered mammalian cell of claim 1, wherein the engineered mammalian cell expresses a mutant EF-2 protein.

7. The engineered mammalian cell of claim 6, wherein the mutant EF-2 has a G→A mutation in the first nucleotide of codon 717 of the protein coding sequence.

8. The engineered mammalian cell of claim 1, wherein the engineered mammalian cell comprises an exogenous nucleic acid sequence that encodes for a PE.

9.-10. (canceled)

11. The engineered mammalian cell of claim 1, wherein the expressed PE is a fusion protein.

12. The engineered mammalian cell of claim 11, wherein the fusion protein further comprises interleukin 13 (IL-13).

13. The engineered mammalian cell of claim 11, wherein the fusion protein further comprises epidermal growth factor (EGF).

14. (canceled)

15. The engineered mammalian cell of claim 1, wherein the mammalian cell is a stem cell or a somatic cell.

16. (canceled)

17. A composition comprising of engineered mammalian cells of claim 1.

18.-34. (canceled)

35. A method of making an engineered mammalian cell that is resistant to Pseudonomas exotoxin comprising:
a. contacting a mammalian cell with an oligonucleotide comprising SEQ. ID. No: 1 that confers a G→A mutation in the first nucleotide of codon 717 of a coding sequence of an elongation factor 2 protein (EF-2);
b. culturing the mammalian cell of step (a) in the presence of a ADP-ribosylating toxin for a period of time; and
c. selecting for the mammalian cell of step (b) that has formed a single colony in the presence of the ADP-ribosylating toxin.

36. The method of claim 35, the method further comprising:
d. contacting the colony of cells of step (c) with a vector comprising an exogenous nucleic acid sequence encoding for a Pseudonomas exotoxin (PE);
e. culturing the contacted cell of step (d) for a period of time; and
f. selecting for the contacted cell of step (d) for the expression of PE.

37. (canceled)

38. The method of claim 36, the exogenous nucleic acid sequence encodes a fusion PE.

39. The method of claim 38, the exogenous nucleic acid sequence encodes a fusion PE that is destined for extracellular secretion.

40. The method of claim 36, the exogenous nucleic acid sequence comprising sequence selected from the group consisting of SEQ. ID. No: 2-4.

41. The method of claim 35, the ADP-ribosylating toxin is diphtheria toxin (DT) or PE.

42. A method of making a self-renewing engineered mammalian cell that secretes Pseudonomas exotoxin comprising:
a. contacting a mammalian cell that is resistant to a Pseudonomas exotoxin (PE) with a vector comprising an exogenous nucleic acid sequence encoding for a Pseudonomas exotoxin (PE);
b. culturing the contacted cell of step (a) for a period of time; and
c. selecting for the cell of step (b) for the expression of PE.

43.-49. (canceled)

50. A method of treating cancer in a subject comprising administering an effective amount of engineered mammalian cells of claim 1 into the subject.

51.-59. (canceled)

60. The method of claim 50, wherein the cancer is selected from a group consisting of brain tumors-glioblastoma multiforme (GBM), melanoma, breast cancer, and lung cancer.

61.-62. (canceled)

63. A method of inducing apoptosis in a tumor in a subject comprising:
a. resectioning the tumor from the subject; and
b. implanting a population of engineered mammalian cells of claim 1 into the resection site produced in step (a).

64.-71. (canceled)


SEQUENCE LISTING (24 sequences)

SEQ ID NO: 1 - DNA, 44 bp, Homo sapiens
tgtggggatctggccccctctgcggtggatggcgtcggcgtgca

SEQ ID NO: 2 - DNA, 361 bp, Pseudomonas sp.
atcgacaacgccctcagcatcaccagcgacggcctgaccatccgcctcgaaggtggcgtcgagccgaacaagccggtgcgctacagctacacgcgccaggcgcgcggcagttggtcgctgaactggctggtgccgatcggccacgagaagccttcgaacatcaaggtgttcatccacgaactgaacgccggtaaccagctcagccacatgtcgccgatctacaccatcgagatgggcgacgagttgctggcgaagctggcgcgcgatgccaccttcttcgtcagggcgcacgagagcaacgagatgcagccgacgctcgccatcagccatgccggggtcagcgtggtcatggcccaggccc

SEQ ID NO: 3 - DNA, 1374 bp, Artificial Sequence
  Description of Artificial Sequence Synthetic polynucleotide
  modified_base (132)..(132): a, c, t, g, unknown or other
atgacagtgctggcgccagcctggagcccaacaacctatctcctcctgctgctgctgctgagctcgggactcagtgggtcagatccgctagcatgacagtgctggcgccagcctggagcccaacaacctatntcctcctgctgctgctgctgagctcgggactcagtggggaattccaggtaaagctggaggagtctgggggagggtcggtgcagactgggggctctctgagactcacctgtgcagcctctggacgcacttcaaggagctatggcatgggatggttccgccaggctccagggaaggagcgtgagtttgtatcaggtattagttggaggggtgatagtacaggctatgcagactccgtgaagggccgattcaccatctccagagacaacgccaagaacacggtggatctgcaaatgaacagcctgaaacctgaggacacggccatttattattgtgcggcggccgcaggtagtgcctggtacggaacattgtatgaatacgactactggggccaggggacccaggtcaccgtctcctcaggcggtggaggcagcggtggcgggggatccgaggtgcagctggtggagtctgggggaggattggtgcaggctgggggctctctgagactctcctgtgcagcctctggacgcaccttcagtagctatgccatgggctggttccgccaggctccagggaaggagcgtgagtttgtagtagctattaactggagtagtggtagcacatactatgcagactccgtgaagggccgattcaccatctccagagacaacgccaagaacacgatgtatctgcagatgaacagcctgaaacctgaggacacggccgtttattactgtgcagcagggtatcagattaatagtggtaattacaactttaaagactatgagtatgactactggggccaggggacccaggtcaccgtctcctcagatatcggatctaccggtggatctggaaaacccggatctggagagggatctaccatcgacaacgccctcagcatcaccagcgacggcctgaccatccgcctcgaaggtggcgtcgagccgaacaagccggtgcgctacagctacacgcgccaggcgcgcggcagttggtcgctgaactggctggtgccgatcggccacgagaagccttcgaacatcaaggtgttcatccacgaactgaacgccggtaaccagctcagccacatgtcgccgatctacaccatcgagatgggcgacgagttgctggcgaagctggcgcgcgatgccaccttcttcgtcagggcgcacgagagcaacgagatgcagccgacgctcgccatcagccatgccggggtcagcgtggtcatggcccaggccc

SEQ ID NO: 4 - DNA, 932 bp, Artificial Sequence
  Description of Artificial Sequence Synthetic polynucleotide
atgacagtgctggcgccagcctggagcccaacaacctatctcctcctgctgctgctgctgagctcgggactcagtgggatggcgcttttgttgaccacggtcattgctctcacttgccttggcggctttgcctccccaggccctgtgcctccctctacagccctcagggagctcattgaggagctggtcaacatcacccagaaccagaaggctccgctctgcaatggcagcatggtatggagcatcaacctgacagctggcatgtactgtgcagccctggaatccctgatcaacgtgtcaggctgcagtgccatcgagaagacccagaggatgctgagcggattctgcccgcacaaggtctcagctgggcagttttccagcttgcatgtccgagacaccaaaatcgaggtggcccagtttgtaaaggacctgctcttacatttaaagaaactttttcgcgagggacggttcaactgaaacttcgaaagcatcattatttgcagagacaggacctgacgatatcggatctaccggtggatctggaaaacccggatctggagagggatctaccatcgacaacgccctcagcatcaccagcgacggcctgaccatccgcctcgaaggtggcgtcgagccgaacaagccggtgcgctacagctacacgcgccaggcgcgcggcagttggtcgctgaactggctggtgccgatcggccacgagaagccttcgaacatcaaggtgttcatccacgaactgaacgccggtaaccagctcagccacatgtcgccgatctacaccatcgagatgggcgacgagttgctggcgaagctggcgcgcgatgccaccttcttcgtcagggcgcacgagagcaacgagatgcagccgacgctcgccatcagccatgccggggtcagcgtggtcatggcccaggccc

SEQ ID NO: 5 - protein, 858 aa, Homo sapiens
MVNFTVDQIRAIMDKKANIRNMSVIAHVDHGKSTLTDSLVCKAGIIASARAGETRFTDTRKDEQERCITIKSTAISLFYELSENDLNFIKQSKDGAGFLINLIDSPGHVDFSSEVTAALRVTDGALVVVDCVSGVCVQTETVLRQAIAERIKPVLMMNKMDRALLELQLEPEELYQTFQRIVENVNVIISTYGEGESGPMGNIMIDPVLGTVGFGSGLHGWAFTLKQFAEMYVAKFAAKGEGQLGPAERAKKVEDMMKKLWGDRYFDPANGKFSKSATSPEGKKLPRTFCQLILDPIFKVFDAIMNFKKEETAKLIEKLDIKLDSEDKDKEGKPLLKAVMRRWLPAGDALLQMITIHLPSPVTAQKYRCELLYEGPPDDEAAMGIKSCDPKGPLMMYISKMVPTSDKGRFYAFGRVFSGLVSTGLKVRIMGPNYTPGKKEDLYLKPIQRTILMMGRYVEPIEDVPCGNIVGLVGVDQFLVKTGTITTFEHAHNMRVMKFSVSPVVRVAVEAKNPADLPKLVEGLKRLAKSDPMVQCIIEESGEHIIAGAGELHLEICLKDLEEDHACIPIKKSDPVVSYRETVSEESNVLCLSKSPNKHNRLYMKARPFPDGLAEDIDKGEVSARQELKQRARYLAEKYEWDVAEARKIWCFGPDGTGPNILTDITKGVQYLNEIKDSVVAGFQWATKEGALCEENMRGVRFDVHDVTLHADAIHRGGGQIIPTARRCLYASVLTAQPRLMEPIYLVEIQCPEQVVGGIYGVLNRKRGHVFEESQVAGTPMFVVKAYLPVNESFGFTADLRSNTGGQAFPQCVFDHWQILPGDPFDNSSRPSQVVAETRKRKGLKEGIPALDNFLDKL

SEQ ID NO: 6 - DNA, 2577 bp, Homo sapiens
atggtgaacttcacggtagaccagatccgcgccatcatggacaagaaggccaacatccgcaacatgtctgtcatcgcccacgtggaccatggcaagtccacgctgacagactccctggtgtgcaaggcgggcatcatcgcctcggcccgggccggggagacacgcttcactgatacccggaaggacgagcaggagcgttgcatcaccatcaagtcaactgccatctccctcttctacgagctctcggagaatgacttgaacttcatcaagcagagcaaggacggtgccggcttcctcatcaacctcattgactcccccgggcatgtcgacttctcctcggaggtgactgctgccctccgagtcaccgatggcgcattggtggtggtggactgcgtgtcaggcgtgtgcgtgcagacggagacagtgctgcggcaggccattgccgagcgcatcaagcctgtgctgatgatgaacaagatggaccgcgccctgctggagctgcagctggagcccgaggagctctaccagactttccagcgcatcgtggagaacgtgaacgtcatcatctccacctacggcgagggcgagagcggccccatgggcaacatcatgatcgatcctgtcctcggtaccgtgggctttgggtctggcctccacgggtgggccttcaccctgaagcagtttgccgagatgtatgtggccaagttcgccgccaagggggagggccagttggggcctgccgagcgggccaagaaagtagaggacatgatgaagaagctgtggggtgacaggtactttgacccagccaacggcaagttcagcaagtcagccaccagccccgaagggaagaagctgccacgcaccttctgccagctgatcctggaccccatcttcaaggtgtttgatgcgatcatgaatttcaagaaagaggagacagcaaaactgatagagaaactggacatcaaactggacagcgaggacaaggacaaagaaggcaaacccctgctgaaggctgtgatgcgccgctggctgcctgccggagacgccttgttgcagatgatcaccatccacctgccctcccctgtgacggcccagaagtaccgctgcgagctcctgtacgaggggcccccggacgacgaggctgccatgggcattaaaagctgtgaccccaaaggccctcttatgatgtatatttccaaaatggtgccaacctccgacaaaggtcggttctacgcctttggacgagtcttctcggggctggtctccactggcctgaaggtcaggatcatggggcccaactatacccctgggaagaaggaggacctctacctgaagccaatccagagaacaatcttgatgatgggccgctacgtggagcccatcgaggatgtgccttgtgggaacattgtgggcctcgtgggcgtggaccagttcctggtgaagacgggcaccatcaccaccttcgagcacgcgcacaacatgcgggtgatgaagttcagcgtcagccctgttgtcagagtggccgtggaggccaagaacccggctgacctgcccaagctggtggaggggctgaagcggctggccaagtccgaccccatggtgcagtgcatcatcgaggagtcgggagagcatatcatcgcgggcgccggcgagctgcacctggagatctgcctgaaggacctggaggaggaccacgcctgcatccccatcaagaaatctgacccggtcgtctcgtaccgcgagacggtcagtgaagagtcgaacgtgctctgcctctccaagtcccccaacaagcacaaccggctgtacatgaaggcgcggcccttccccgacggcctggccgaggacatcgataaaggcgaggtgtccgcccgtcaggagctcaagcagcgggcgcgctacctggccgagaagtacgagtgggacgtggctgaggcccgcaagatctggtgctttgggcccgacggcaccggccccaacatcctcaccgacatcaccaagggtgtgcagtacctcaacgagatcaaggacagtgtggtggccggcttccagtgggccaccaaggagggcgcactgtgtgaggagaacatgcggggtgtgcgcttcgacgtccacgacgtcaccctgcacgccgacgccatccaccgcggagggggccagatcatccccacagcacggcgctgcctctatgccagtgtgctgaccgcccagccacgcctcatggagcccatctaccttgtggagatccagtgtccagagcaggtggtcggtggcatctacggggttttgaacaggaagcggggccacgtgttcgaggagtcccaggtggccggcacccccatgtttgtggtcaaggcctatctgcccgtcaacgagtcctttggcttcaccgctgacctgaggtccaacacgggcggccaggcgttcccccagtgtgtgtttgaccactggcagatcctgcccggagaccccttcgacaacagcagccgccccagccaggtggtggcggagacccgcaagcgcaagggcctgaaagaaggcatccctgccctggacaacttcctggacaaattgtag

SEQ ID NO: 7 - DNA, 2577 bp, Homo sapiens
atggtgaacttcacggtagaccagatccgcgccatcatggacaagaaggccaacatccgcaacatgtctgtcatcgcccacgtggaccatggcaagtccacgctgacagactccctggtgtgcaaggcgggcatcatcgcctcggcccgggccggggagacacgcttcactgatacccggaaggacgagcaggagcgttgcatcaccatcaagtcaactgccatctccctcttctacgagctctcggagaatgacttgaacttcatcaagcagagcaaggacggtgccggcttcctcatcaacctcattgactcccccgggcatgtcgacttctcctcggaggtgactgctgccctccgagtcaccgatggcgcattggtggtggtggactgcgtgtcaggcgtgtgcgtgcagacggagacagtgctgcggcaggccattgccgagcgcatcaagcctgtgctgatgatgaacaagatggaccgcgccctgctggagctgcagctggagcccgaggagctctaccagactttccagcgcatcgtggagaacgtgaacgtcatcatctccacctacggcgagggcgagagcggccccatgggcaacatcatgatcgatcctgtcctcggtaccgtgggctttgggtctggcctccacgggtgggccttcaccctgaagcagtttgccgagatgtatgtggccaagttcgccgccaaaggggagggccagttggggcctgccgagcgggccaagaaagtagaggacatgatgaagaagctgtggggtgacaggtactttgacccagccaacggcaagttcagcaagtcagccaccagccccgaagggaagaagctgccacgcaccttctgccagctgatcctggaccccatcttcaaggtgtttgatgcgatcatgaatttcaagaaagaggagacagcaaaactgatagagaaactggacatcaaactggacagcgaggacaaggacaaagaaggcaaacccctgctgaaggctgtgatgcgccgctggctgcctgccggagacgccttgttgcagatgatcaccatccacctgccctcccctgtgacggcccagaagtaccgctgcgagctcctgtacgaggggcccccggacgacgaggctgccatgggcattaaaagctgtgaccccaaaggccctcttatgatgtatatttccaaaatggtgccaacctccgacaaaggtcggttctacgcctttggacgagtcttctcggggctggtctccactggcctgaaggtcaggatcatggggcccaactatacccctgggaagaaggaggacctctacctgaagccaatccagagaacaatcttgatgatgggccgctacgtggagcccatcgaggatgtgccttgtgggaacattgtgggcctcgtgggcgtggaccagttcctggtgaagacgggcaccatcaccaccttcgagcacgcgcacaacatgcgggtgatgaagttcagcgtcagccctgttgtcagagtggccgtggaggccaagaacccggctgacctgcccaagctggtggaggggctgaagcggctggccaagtccgaccccatggtgcagtgcatcatcgaggagtcgggagagcatatcatcgcgggcgccggcgagctgcacctggagatctgcctgaaggacctggaggaggaccacgcctgcatccccatcaagaaatctgacccggtcgtctcgtaccgcgagacggtcagtgaagagtcgaacgtgctctgcctctccaagtcccccaacaagcacaaccggctgtacatgaaggcgcggcccttccccgacggcctggccgaggacatcgataaaggcgaggtgtccgcccgtcaggagctcaagcagcgggcgcgctacctggccgagaagtacgagtgggacgtggctgaggcccgcaagatctggtgctttgggcccgacggcaccggccccaacatcctcaccgacatcaccaagggtgtgcagtacctcaacgagatcaaggacagtgtggtggccggcttccagtgggccaccaaggagggcgcactgtgtgaggagaacatgcggggtgtgcgcttcgacgtccacgacgtcaccctgcacgccgacgccatccaccgcggagggggccagatcatccccacagcacggcgctgcctctatgccagtgtgctgaccgcccagccacgcctcatggagcccatctaccttgtggagatccagtgtccagagcaggtggtcggtggcatctacggggttttgaacaggaagcggggccacgtgttcgaggagtcccaggtggccggcacccccatgtttgtggtcaaggcctatctgcccgtcaacgagtcctttggcttcaccgctgacctgaggtccaacacgggcggccaggcgttcccccagtgtgtgtttgaccactggcagatcctgcccggagaccccttcgacaacagcagccgccccagccaggtggtggcggagacccgcaagcgcaagggcctgaaagaaggcatccctgccctggacaacttcctggacaaattgtag

SEQ ID NO: 8 - DNA, 78 bp, Homo sapiens
atgacagtgctggcgccagcctggagcccaacaacctatctcctcctgctgctgctgctgagctcgggactcagtggg

SEQ ID NO: 9 - DNA, 23 bp, Artificial Sequence
  Description of Artificial Sequence Synthetic primer
atggctttcgtttgcttggctat

SEQ ID NO: 10 - DNA, 24 bp, Artificial Sequence
  Description of Artificial Sequence Synthetic primer
tcatgtatcacagaaaaattctgg

SEQ ID NO: 11 - DNA, 20 bp, Artificial Sequence
  Description of Artificial Sequence Synthetic primer
gtcagtggtggacctgacct

SEQ ID NO: 12 - DNA, 20 bp, Artificial Sequence
  Description of Artificial Sequence Synthetic primer
tgctgtagccaaattcgttg

SEQ ID NO: 13 - DNA, 21 bp, Artificial Sequence
  Description of Artificial Sequence Synthetic primer
gaacccgacgcacgcggccgg

SEQ ID NO: 14 - DNA, 29 bp, Artificial Sequence
  Description of Artificial Sequence Synthetic primer
ccgctcgagcttcaggtcctcgcgcggcg

SEQ ID NO: 15 - DNA, 20 bp, Artificial Sequence
  Description of Artificial Sequence Synthetic primer
gaatcagagaagacaggcca

SEQ ID NO: 16 - DNA, 20 bp, Artificial Sequence
  Description of Artificial Sequence Synthetic primer
gtgtaggtatcataactccg

SEQ ID NO: 17 - DNA, 881 bp, Pseudomonas sp.
  modified_base (54)..(54): a, c, t, g, unknown or other
tcagatccgctagcatgacagtgctggcgccagcctggagcccaacaacctatntcctcctgctgctgctgctgagctcgggactcagtggggaattccaggtaaagctggaggagtctgggggagggtcggtgcagactgggggctctctgagactcacctgtgcagcctctggacgcacttcaaggagctatggcatgggatggttccgccaggctccagggaaggagcgtgagtttgtatcaggtattagttggaggggtgatagtacaggctatgcagactccgtgaagggccgattcaccatctccagagacaacgccaagaacacggtggatctgcaaatgaacagcctgaaacctgaggacacggccatttattattgtgcggcggccgcaggtagtgcctggtacggaacattgtatgaatacgactactggggccaggggacccaggtcaccgtctcctcaggcggtggaggcagcggtggcgggggatccgaggtgcagctggtggagtctgggggaggattggtgcaggctgggggctctctgagactctcctgtgcagcctctggacgcaccttcagtagctatgccatgggctggttccgccaggctccagggaaggagcgtgagtttgtagtagctattaactggagtagtggtagcacatactatgcagactccgtgaagggccgattcaccatctccagagacaacgccaagaacacgatgtatctgcagatgaacagcctgaaacctgaggacacggccgtttattactgtgcagcagggtatcagattaatagtggtaattacaactttaaagactatgagtatgactactggggccaggggacccaggtcaccgtctcctca

SEQ ID NO: 18 - DNA, 439 bp, Pseudomonas sp.
atggcgcttttgttgaccacggtcattgctctcacttgccttggcggctttgcctccccaggccctgtgcctccctctacagccctcagggagctcattgaggagctggtcaacatcacccagaaccagaaggctccgctctgcaatggcagcatggtatggagcatcaacctgacagctggcatgtactgtgcagccctggaatccctgatcaacgtgtcaggctgcagtgccatcgagaagacccagaggatgctgagcggattctgcccgcacaaggtctcagctgggcagttttccagcttgcatgtccgagacaccaaaatcgaggtggcccagtttgtaaaggacctgctcttacatttaaagaaactttttcgcgagggacggttcaactgaaacttcgaaagcatcattatttgcagagacaggacctgac

SEQ ID NO: 19 - DNA, 1086 bp, Pseudomonas sp.
ggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacgccgacgtggtgagcctgacctgcccggtcgccgccggtgaatgcgcgggcccggcggacagcggcgacgccctgctggagcgcaactatcccactggcgcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgcgcgaggacctgaagtaa

SEQ ID NO: 20 - protein, 361 aa, Pseudomonas sp.
GGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 21 - protein, 638 aa, Pseudomonas aeruginosa
MHLTPHWIPLVASLGLLAGGSFASAAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 22 - DNA, 1917 bp, Pseudomonas aeruginosa
atgcacctgataccccattggatccccctggtcgccagcctcggcctgctcgccggcggctcgtccgcgtccgccgccgaggaagccttcgacctctggaacgaatgcgccaaagcctgcgtgctcgacctcaaggacggcgtgcgttccagccgcatgagcgtcgacccggccatcgccgacaccaacggccagggcgtgctgcactactccatggtcctggagggcggcaacgacgcgctcaagctggccatcgacaacgccctcagcatcaccagcgacggcctgaccatccgcctcgaaggcggcgtcgagccgaacaagccggtgcgctacagctacacgcgccaggcgcgcggcagttggtcgctgaactggctggtaccgatcggccacgagaagccctcgaacatcaaggtgttcatccacgaactgaacgccggcaaccagctcagccacatgtcgccgatctacaccatcgagatgggcgacgagttgctggcgaagctggcgcgcgatgccaccttcttcgtcagggcgcacgagagcaacgagatgcagccgacgctcgccatcagccatgccggggtcagcgtggtcatggcccagacccagccgcgccgggaaaagcgctggagcgaatgggccagcggcaaggtgttgtgcctgctcgacccgctggacggggtctacaactacctcgcccagcaacgctgcaacctcgacgatacctgggaaggcaagatctaccgggtgctcgccggcaacccggcgaagcatgacctggacatcaaacccacggtcatcagtcatcgcctgcactttcccgagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacgccgacgtggtgagcctgacctgcccggtcgccgccggtgaatgcgcgggcccggcggacagcggcgacgccctgctggagcgcaactatcccactggcgcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgcgcgaggacctgaagtaa

SEQ ID NO: 23 - protein, 120 aa, Pseudomonas sp.
IDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQA

SEQ ID NO: 24 - protein, 6 aa, Artificial Sequence
  Description of Artificial Sequence Synthetic 6xHis tag
HHHHHH